(19) United States
(12) Patent Application Publication
(10) Pub. No.: US 2026/0053558 A1
(43) Pub. Date: Feb. 26, 2026

(54) SYSTEM AND METHOD FOR NON-INVASIVE PREDICTION OF PULMONARY VEIN RECONNECTION POST-ATRIAL FIBRILLATION ABLATION USING BODY SURFACE ELECTROCARDIOGRAMS

(71) Applicant: Anumana, Inc., Cambridge, MA (US)

(72) Inventors: Suthirth Vaidya, Bengaluru (IN); Rakesh Barve, Bengaluru (IN); Abhijith Chunduru, Bengaluru (IN); Murali Aravamudan, Andover, MA (US); Animesh Agarwal, San Mateo, CA (US); Samir Awasthi, Boston, MA (US); Maulik Nanavaty, Cambridge, MA (US); Sai Saketh Chennamsetty, Bengaluru (IN); Arjun Puranik, San Jose, CA (US); Harish Kumar B V, Salem (IN)

(73) Assignee: Anumana, Inc., Cambridge, MA (US)

(21) Appl. No.: 19/304,333

(22) Filed: Aug. 19, 2025

Related U.S. Application Data
(63) Continuation-in-part of application No. 19/063,986, filed on Feb. 26, 2025.
Application No. 19/063,986 is a continuation-in-part of application No. 18/810,128, filed on Aug. 20, 2024, now Pat. No. 12,266,107.

(51) Int. Cl. A61B 18/14; A61B 18/00; A61B 34/10; G06N 20/00

(52) U.S. Cl. CPC A61B 18/1492; A61B 34/10; G06N 20/00; A61B 2018/00404; A61B 2018/00577; A61B 2018/00642; A61B 2018/00666; A61B 2018/0069; A61B 2018/00839; A61B 2034/104

(57) ABSTRACT

An apparatus for prediction of repeat ablation efficacy, the apparatus including an electrocardiogram device, wherein the electrocardiogram device is configured to detect post-ablation arrhythmic electrocardiogram (ECG) data representative of a post-ablation arrythmia of a patient who has previously undergone an ablation procedure and a processor configured to receive, from the electrocardiogram device, the post-ablation arrhythmic ECG data predict, using a repeat-ablation efficacy machine-learning model, a determination of a pulmonary vein reconnection by identifying features within the post-ablation arrhythmic ECG data that are historically representative of pulmonary vein reconnection determinations and transmit, for display, the determination.

### CROSS-REFERENCE TO RELATED APPLICATIONS

This application is a continuation-in-part of U.S. Non-Provisional patent application Ser. No. 19/063,986 filed on Feb. 26, 2025, entitled “SYSTEM AND METHOD FOR RESPONDING TO A USER INPUT USING AN AGENT ORCHESTRATOR,” which is a continuation-in-part of U.S. Non-Provisional patent application Ser. No. 18/810,128, filed on Aug. 20, 2024, now U.S. Pat. No. 12,266,107, issued Apr. 1, 2025, and entitled “SYSTEM AND METHOD FOR RESPONDING TO A USER INPUT USING AN AGENT ORCHESTRATOR,” the entirety of each of which are incorporated herein by reference.

### FIELD OF THE INVENTION

The present invention generally relates to the field of machine learning. In particular, the present invention is directed to a system and method for responding to a user input using an agent orchestrator.

### BACKGROUND

Atrial fibrillation (AFib) is commonly treated with ablation procedures designed to electrically isolate the pulmonary veins (PVs) from the rest of the heart. Despite their effectiveness, these procedures are not always permanently successful, as reconnection of the PVs frequently leads to AFib recurrence. At present, confirming such reconnection typically requires invasive procedures involving intracardiac electrograms. There is a need for a non-invasive, predictive tool to identify whether pulmonary vein reconnection has occurred following ablation.

### SUMMARY OF THE DISCLOSURE

In an aspect, an apparatus for prediction of repeat ablation efficacy is described. The apparatus includes an electrocardiogram device, wherein the electrocardiogram device is configured to detect post-ablation arrhythmic electrocardiogram (ECG) data representative of a post-ablation arrythmia of a patient who has previously undergone an ablation procedure. The apparatus further includes at least a processor and a memory communicatively connected to the at least a processor. The memory contains instructions configuring the at least a processor to receive, from the electrocardiogram device, the post-ablation arrhythmic ECG data predict, using a repeat-ablation efficacy machine-learning model, a determination of a pulmonary vein reconnection by identifying features within the post-ablation arrhythmic ECG data that are historically representative of pulmonary vein reconnection determinations and transmit, for display, the determination.

In another aspect, a method for prediction of repeat ablation efficacy is described. The method includes receiving, by at least a processor and from an electrocardiogram device, post-ablation arrhythmic electrocardiogram (ECG) data, wherein the electrocardiogram device is configured to detect the post-ablation arrhythmic ECG data representative of a post-ablation arrythmia of a patient who has previously undergone an ablation procedure, predicting, by at the least a processor and using a repeat-ablation efficacy machine-learning model, a determination of a pulmonary vein reconnection by identifying features within the post-ablation arrhythmic ECG data that are historically representative of pulmonary vein reconnection determinations and transmitting, by the at least a processor and for display, the determination.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram depicting an exemplary embodiment of a system for responding to a user input using an agent orchestrator;

FIG. 2 is a diagram depicting an exemplary embodiment of a system for including an agent orchestrator;

FIG. 3 is a flow diagram of an exemplary embodiment of an ICE image example generation process;

FIG. 4 is a block diagram of an exemplary embodiment of a machine learning model;

FIG. 5 is a schematic diagram of an exemplary embodiment of a neural network;

FIG. 6 is a schematic diagram of an exemplary embodiment of a neural network node;

FIG. 7 is an illustration of an exemplary operatory during an electrophysiology procedure using an exemplary stylized electrophysiology copilot;

FIG. 8 is an illustration of an exemplary embodiment of an interface of an electrophysiology copilot;

FIG. 9 includes 2 illustrations of exemplary embodiments of an interface of an electrophysiology copilot;

FIG. 10 is a flow diagram depicting an exemplary embodiment of a method of responding to a user input using an agent orchestrator;

FIG. 11 is a diagram of an exemplary embodiment of an apparatus for prediction of repeat ablation efficacy;

FIG. 12 illustrates an exemplary embodiment of an ECG;

FIG. 13 is a flow diagram illustrating an exemplary method for prediction of pulmonary vein reconnection; and

FIG. 14 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for responding to a user input using an agent orchestrator. A system may receive, from a user interface, a user input such as text or speech. A system may, using an agent orchestrator, select an appropriate agent. Such agent may receive additional data, such as procedure data or electronic health record data, as an input, and may produce an output which is responsive to the user input.

Aspects of the present disclosure involve the use of body surface ECG signals collected post-ablation to predict whether pulmonary vein reconnection has occurred. By analyzing certain patterns in the surface ECG, a predictive model (such as a machine learning-based model, such as post-ablation efficacy machine learning model 1113) may infer the electrical connectivity status of the PVs without invasive instrumentation.

Aspects of the present disclosure include a non-invasive approach for assessing pulmonary vein (PV) reconnection following atrial fibrillation (AFib) ablation procedures. Specifically, aspects s of the present disclosure may utilize body surface electrocardiogram (ECG) signals collected after ablation to predict whether PV reconnection has occurred. By analyzing distinct patterns in these surface ECGs, a predictive model, powered by machine learning consistent with the below disclosure, can infer the electrical connectivity status of the PVs without the need for invasive intracardiac instrumentation. Aspects of the present disclosure may be implemented in conjunction with various post-ablation procedures and can further be implemented with broader systems as well as described herein.

In one or more embodiments, aspects of the present disclosure include the development of a predictive algorithm trained on datasets correlating post-ablation body surface ECGs with a ground-truth status of PV reconnection, as verified through electrograms or clinical outcomes. Additional aspects involve identification of surface ECG biomarkers indicative of PV reconnection and integration of a decision-support tool that classifies patients into those with likely PV reconnection, who may be candidates for reablation and those with likely persistent PV isolation, for whom alternative treatment strategies should be considered.

In one or more embodiments, aspects of the present disclosure may be used in conjunction with patient treatment options. These include, but are not limited to, post-ablation monitoring through follow-up ECGs to detect reconnection without requiring repeat invasive diagnostics, guiding reablation triage by identifying patients who may benefit from a second procedure, and stratifying patients in clinical trials based on their PV reconnection status to better assess treatment outcomes.

Aspects of the present disclosure may form part of a broader input-output modeling framework aimed at improving outcomes in AFib ablation. Inputs to this framework may include post-ablation body surface ECG recordings (potentially serial over time), as well as optional supplementary data such as patient history, prior electrogram (EGM) data, and procedural metadata. The outputs may include binary or probabilistic predictions (e.g., “PV reconnection likely” or “PV reconnection unlikely”) and corresponding clinical recommendations (e.g., “Reablation advised” or “Reablation not advised”).

The model training process as described herein may utilize ground truth reconnection status determined via electrograms or patterns of clinical recurrence. The predictive model May employ statistical methods or deep learning techniques to detect and interpret ECG features most relevant to PV reconnection.

Aspects of the present disclosure directed toward a mid-level outcome within a hierarchical framework of AFib ablation evaluation. long-term AFib recurrence, a complex and longitudinal endpoint may be situated at the highest level. Assessment of individual lesion efficacy may occur at the lowest level. In one or more embodiments, aspects of the present disclosure may focus on an intermediate outcome which includes determining whether functional PV isolation achieved during the initial procedure remains intact over time.

Compared to existing solutions, aspects of the present disclosure offer multiple advantages: they are non-invasive, avoiding the need for costly and risky catheterization; timely, enabling use during routine post-procedural follow-up; personalized, accommodating individual variations in ECG signatures; and scalable, making widespread, decentralized monitoring feasible even outside tertiary care centers.

As described in greater detail below, aspects of the present disclosure may be implemented in alignment with additional system components, model design elements, and clinical integration strategies that collectively support predictive, patient-specific decision-making in the management of AFib ablation outcomes.

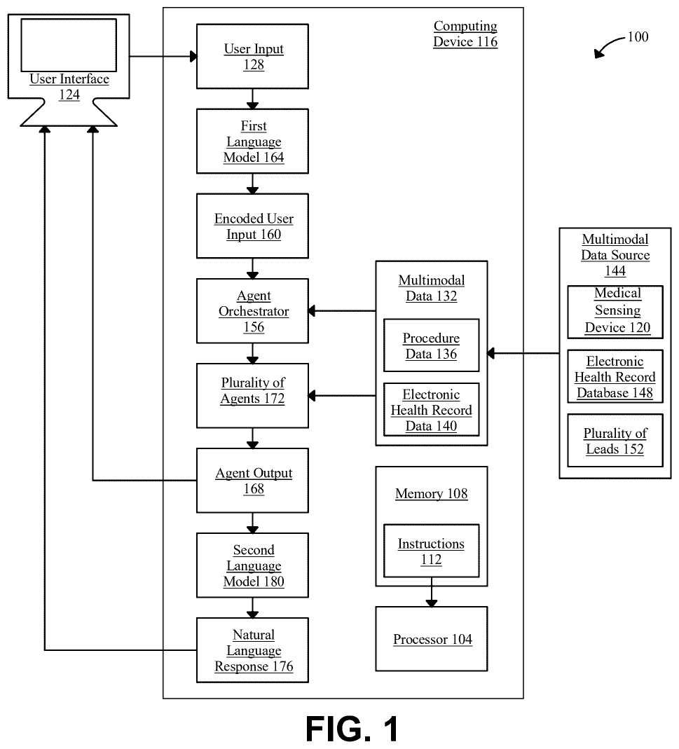

Referring now to FIG. 1, an exemplary embodiment of a system 100 for responding to a user input using an agent orchestrator is illustrated. System 100 may include a computing device. System 100 may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1, in some embodiments, system 100 may include at least a processor 104 and a memory 108 communicatively connected to the at least a processor 104, the memory 108 containing instructions 112 configuring the at least a processor 104 to perform one or more processes described herein. Computing device 116 may include processor 104 and/or memory 108. Computing device 116 may be configured to perform one or more processes described herein.

Still referring to FIG. 1, computing device 116 may include but is not limited to, for example, a computing device or cluster of computing devices in a first location and a second computing device or cluster of computing devices in a second location. Computing device 116 may include one or more computing devices dedicated to data storage, security, distribution of traffic for load balancing, and the like. Computing device 116 may distribute one or more computing tasks as described below across a plurality of computing devices of computing device, which may operate in parallel, in series, redundantly, or in any other manner used for distribution of tasks or memory between computing devices. Computing device 116 may be implemented, as a non-limiting example, using a “shared nothing” architecture.

Still referring to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, system 100 includes a medical sensing device 120. As used in this disclosure, a “medical sensing device” is a device configured to detect, measure, or monitor physiological signal of a subject. a medical sensing device 120, wherein the medical sensing device 120 is configured to detect procedure data as described herein. The medical sensing device 120 may include, without limitation, wearable sensors, implantable devices, diagnostic instruments, or remote monitoring systems. The medical sensing device 120 may utilize various sensing modalities, such as optical, electrical, mechanical, acoustic, or biochemical detection, to collect data relevant to patient health, diagnosis, or treatment. The medical sensing device 120 may further include communication capabilities to transmit sensed data to an external system, such as a healthcare provider, medical database, or artificial intelligence system for analysis.

Still referring to FIG. 1, in some embodiments, system 100 may include a catheter. As used herein, a “catheter” is a medical device comprising a tube which may be inserted into a passageway of a subject. In some embodiments, catheter may include a cardiac catheter. As used herein, a “cardiac catheter” is a catheter configured for use in the heart. Catheter may be used to, in non-limiting examples, examine and/or replace heart valves, repair heart defects, take samples of blood and/or heart muscle, inject dye into arteries, and/or capture an ultrasonic image. In some embodiments, catheter and/or a process using catheter, such as capturing and/or processing an ultrasonic image, may be consistent with U.S. patent application Ser. No. 18/395,087 (having attorney docket number 1518-110USU1), filed on Dec. 22, 2023, and titled “APPARATUS AND METHOD FOR GENERATING A THREE-DIMENSIONAL (3D) MODEL OF CARDIAC ANATOMY WITH AN OVERLAY,” the entirety of which is hereby incorporated by reference.

Still referring to FIG. 1, in some embodiments, system 100 may include user interface 124. User interface 124 may include, in non-limiting examples, a smartphone, smartwatch, laptop computer, desktop computer, virtual reality device, tablet, and/or a component thereof. User interface 124 may include an input interface and/or an output interface. An input interface may include one or more mechanisms for a computing device to receive data from a user such as, in non-limiting examples, a mouse, keyboard, button, scroll wheel, camera, microphone, switch, lever, touchscreen, trackpad, joystick, and controller. An output interface may include one or more mechanisms for a computing device to output data to a user such as, in non-limiting examples, a screen, speaker, and haptic feedback system. An output interface May be used to display one or more elements of data described herein. As used herein, a device “displays” a datum if the device outputs the datum in a format suitable for communication to a user. For example, a device may display a datum by outputting text or an image on a screen or outputting a sound using a speaker.

Still referring to FIG. 1, in some embodiments, system 100 may receive, using user interface 124, a user input 128. As used herein, a “user interface” is a mechanism by which a user may input information into a computing device, a mechanism by which a computing device may output information to a user, or both. As used herein, a “user input” is a datum generated as a function of an interaction between a user and an input interface. A user input may include, in non-limiting examples, a request by a user for a particular datum and/or another prompt input by a user. In a non-limiting example, a user input may include a request that system 100 display a particular electronic health record of a subject. In another non-limiting example, a user input may include a question as to whether a user's electrocardiogram (ECG) data suggests that a user has a particular cardiac condition. In some embodiments, user input 128 may include text input data. In some embodiments, user input 128 may include audio input data. As used herein, “audio input data” is data representing sound recorded by a sensor. Audio input data may include, in a non-limiting example, a spoken prompt by a user which is captured using a microphone.

Still referring to FIG. 1, in some embodiments, audio input data may be processed using automatic speech recognition. For example, audio input data may be transcribed using an automatic speech recognition process. In some embodiments, automatic speech recognition may require training (i.e., enrollment). In some cases, training an automatic speech recognition model may require an individual speaker to read text or isolated vocabulary. In some cases, audio training data may include an audio component having an audible verbal content, the contents of which are known a priori by a computing device. Computing device may then train an automatic speech recognition model according to training data which includes audible verbal content correlated to known content. In this way, computing device may analyze a person's specific voice and train an automatic speech recognition model to the person's speech, resulting in increased accuracy. Alternatively, or additionally, in some cases, computing device may include an automatic speech recognition model that is speaker independent. As used in this disclosure, a “speaker independent” automatic speech recognition process is an automatic speech recognition process that does not require training for each individual speaker. Conversely, as used in this disclosure, automatic speech recognition processes that employ individual speaker specific training are “speaker dependent.”

Still referring to FIG. 1, in some embodiments, an automatic speech recognition process may perform voice recognition or speaker identification. As used in this disclosure, “voice recognition” is a process of identifying a speaker, from audio content, rather than what the speaker is saying. In some cases, computing device may first recognize a speaker of verbal audio content and then automatically recognize speech of the speaker, for example by way of a speaker dependent automatic speech recognition model or process. In some embodiments, an automatic speech recognition process can be used to authenticate or verify an identity of a speaker. In some cases, a speaker may or may not include subject. For example, subject may speak within audio input data, but others may speak as well.

Still referring to FIG. 1, in some embodiments, an automatic speech recognition process may include one or all of acoustic modeling, language modeling, and statistically based speech recognition algorithms. In some cases, an automatic speech recognition process may employ hidden Markov models (HMMs). As discussed in greater detail below, language modeling such as that employed in natural language processing applications like document classification or statistical machine translation, may also be employed by an automatic speech recognition process.

Still referring to FIG. 1, an exemplary algorithm employed in automatic speech recognition may include or even be based upon hidden Markov models. Hidden Markov models (HMMs) may include statistical models that output a sequence of symbols or quantities. HMMs can be used in speech recognition because a speech signal can be viewed as a piecewise stationary signal or a short-time stationary signal. For example, over a short time scale (e.g., 10 milliseconds), speech can be approximated as a stationary process. Speech (i.e., audible verbal content) can be understood as a Markov model for many stochastic purposes.

Still referring to FIG. 1, in some embodiments HMMs can be trained automatically and may be relatively simple and computationally feasible to use. In an exemplary automatic speech recognition process, a hidden Markov model may output a sequence of n-dimensional real-valued vectors (with n being a small integer, such as 10), at a rate of about one vector every 10 milliseconds. Vectors may consist of cepstral coefficients. A cepstral coefficient requires using a spectral domain. Cepstral coefficients may be obtained by taking a Fourier transform of a short time window of speech yielding a spectrum, decorrelating the spectrum using a cosine transform, and taking first (i.e., most significant) coefficients. In some cases, an HMM may have in each state a statistical distribution that is a mixture of diagonal covariance Gaussians, yielding a likelihood for each observed vector. In some cases, each word, or phoneme, may have a different output distribution; an HMM for a sequence of words or phonemes may be made by concatenating an HMMs for separate words and phonemes.

Still referring to FIG. 1, in some embodiments, an automatic speech recognition process may use various combinations of a number of techniques in order to improve results. In some cases, a large-vocabulary automatic speech recognition process may include context dependency for phonemes. For example, in some cases, phonemes with different left and right context may have different realizations as HMM states. In some cases, an automatic speech recognition process may use cepstral normalization to normalize for different speakers and recording conditions. In some cases, an automatic speech recognition process may use vocal tract length normalization (VTLN) for male-female normalization and maximum likelihood linear regression (MLLR) for more general speaker adaptation. In some cases, an automatic speech recognition process may determine so-called delta and delta-delta coefficients to capture speech dynamics and might use heteroscedastic linear discriminant analysis (HLDA). In some cases, an automatic speech recognition process may use splicing and a linear discriminate analysis (LDA)-based projection, which may include heteroscedastic linear discriminant analysis, or a global semi-tied covariance transform (also known as maximum likelihood linear transform [MLLT]). In some cases, an automatic speech recognition process may use discriminative training techniques, which may dispense with a purely statistical approach to HMM parameter estimation and instead optimize some classification-related measure of training data; examples may include maximum mutual information (MMI), minimum classification error (MCE), and minimum phone error (MPE).

Still referring to FIG. 1, in some embodiments, an automatic speech recognition process may be said to decode speech (i.e., audible verbal content). Decoding of speech may occur when an automatic speech recognition system is presented with a new utterance and must compute a most likely sentence. In some cases, speech decoding may include a Viterbi algorithm. A Viterbi algorithm may include a dynamic programming algorithm for obtaining a maximum a posteriori probability estimate of a most likely sequence of hidden states (i.e., Viterbi path) that results in a sequence of observed events. Viterbi algorithms may be employed in context of Markov information sources and hidden Markov models. A Viterbi algorithm may be used to find a best path, for example using a dynamically created combination hidden Markov model, having both acoustic and language model information, using a statically created combination hidden Markov model (e.g., finite state transducer [FST] approach).

Still referring to FIG. 1, in some embodiments, speech (i.e., audible verbal content) decoding may include considering a set of good candidates and not only a best candidate, when presented with a new utterance. In some cases, a better scoring function (i.e., re-scoring) may be used to rate each of a set of good candidates, allowing selection of a best candidate according to this refined score. In some cases, a set of candidates can be kept either as a list (i.e., N-best list approach) or as a subset of models (i.e., a lattice). In some cases, re-scoring may be performed by optimizing Bayes risk (or an approximation thereof). In some cases, re-scoring may include optimizing for sentence (including keywords) that minimizes an expectancy of a given loss function with regards to all possible transcriptions. For example, re-scoring may allow selection of a sentence that minimizes an average distance to other possible sentences weighted by their estimated probability. In some cases, an employed loss function may include Levenshtein distance, although different distance calculations may be performed, for instance for specific tasks. In some cases, a set of candidates may be pruned to maintain tractability.

Still referring to FIG. 1, in some embodiments, an automatic speech recognition process may employ dynamic time warping (DTW)-based approaches. Dynamic time warping may include algorithms for measuring similarity between two sequences, which may vary in time or speed. For instance, similarities in walking patterns would be detected, even if in one video the person was walking slowly and if in another he or she were walking more quickly, or even if there were accelerations and deceleration during the course of one observation. DTW has been applied to video, audio, and graphics-indeed, any data that can be turned into a linear representation can be analyzed with DTW. In some cases, DTW may be used by an automatic speech recognition process to cope with different speaking (i.e., audible verbal content) speeds. In some cases, DTW may allow computing device to find an optimal match between two given sequences (e.g., time series) with certain restrictions. That is, in some cases, sequences can be “warped” non-linearly to match each other. In some cases, a DTW-based sequence alignment method may be used in context of hidden Markov models.

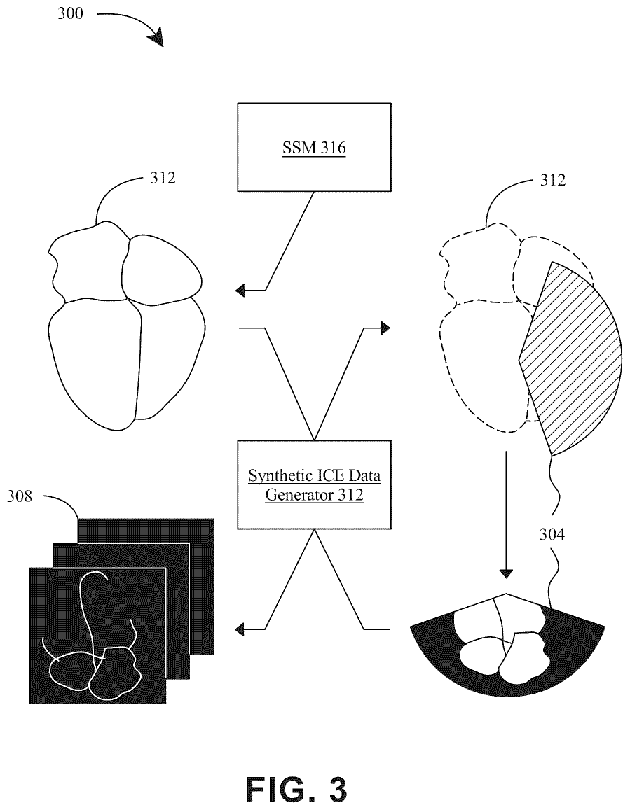

Still referring to FIG. 1, in some embodiments, an automatic speech recognition process may include a neural network. Neural network may include any neural network, for example those disclosed with reference to FIGS. 3-5. In some cases, neural networks may be used for automatic speech recognition, including phoneme classification, phoneme classification through multi-objective evolutionary algorithms, isolated word recognition, audiovisual speech recognition, audiovisual speaker recognition and speaker adaptation. In some cases, neural networks employed in automatic speech recognition may make fewer explicit assumptions about feature statistical properties than HMMs and therefore may have several qualities making them attractive recognition models for speech recognition. When used to estimate the probabilities of a speech feature segment, neural networks may allow discriminative training in a natural and efficient manner. In some cases, neural networks may be used to effectively classify audible verbal content over short-time interval, for instance such as individual phonemes and isolated words. In some embodiments, a neural network may be employed by automatic speech recognition processes for pre-processing, feature transformation and/or dimensionality reduction, for example prior to HMM-based recognition. In some embodiments, long short-term memory (LSTM) and related recurrent neural networks (RNNs) and Time Delay Neural Networks (TDNN's) may be used for automatic speech recognition, for example over longer time intervals for continuous speech recognition.

Still referring to FIG. 1, in some embodiments, system 100 may receive data of one or more modalities, such as multimodal data 132. Data received, such as multimodal data 132, may include in non-limiting examples, procedure data 136 and/or electronic health record data 140. Multimodal data 132 and/or a datum thereof may be received from multimodal data source 144 such as catheter, electronic health record database 148, and/or plurality of leads 152.

Still referring to FIG. 1, in some embodiments, system 100 may receive procedure data 136. As used herein, “procedure data” is data collected in preparation for a medical procedure, data collected during a medical procedure, data collected as a result of a medical procedure, or a combination thereof, where the data describes the subject, the procedure, or both. In some embodiments, procedure data 136 may include cardiac procedure data. As used herein, “cardiac procedure data” is data collected in preparation for a cardiac procedure, data collected during a cardiac procedure, data collected as a result of a cardiac procedure, or a combination thereof, where the data describes the subject, the cardiac procedure, or both. In some embodiments, catheter may be configured to capture procedure data 136. In some embodiments, procedure data 136 may be received from the medical sensing device 120. In some embodiments, procedure data 136 may be received from a sensor of catheter. In some embodiments, cardiac procedure data may be received from a sensor of a cardiac catheter. In some embodiments, procedure data 136 may include an ECG datum. In some embodiments, an ECG datum may be received from plurality of leads 152. In some embodiments, an ECG datum and/or methods of generating, receiving, and/or processing an ECG datum may be consistent with U.S. patent application Ser. No. 18/653,425 (having attorney docket number 1518-145USU1), filed on May 2, 2024, and titled “SYSTEMS AND METHODS FOR SIGNAL DIGITIZATION,” the entirety of which is hereby incorporated by reference. In some embodiments, an ECG datum may include historical ECG data of a subject and/or may be retrieved from electronic health record database 148. In some embodiments, procedure data 136 may include catheter location data. As used herein, “catheter location data” is data describing a location of a catheter, data describing a location of a component of a catheter, or both. In some embodiments, catheter location data may include cardiac catheter location data. As used herein, “cardiac catheter location data” is data describing a location of a cardiac catheter, data describing a location of a component of a cardiac catheter, or both.

With continued reference to FIG. 1, in some embodiments, procedure data 136 may include image data. The image data may be collected from various imaging modalities. The various imaging modalities may provide different insights into anatomical structures and physiological conditions. The selection of a specific imaging modality may depend on the procedure being performed, the level of detail required, and the real-time feedback necessary for guiding interventions. The system may process the image data to enhance decision-making, improve procedural accuracy, and optimize patient outcomes. In some embodiments, the image data may be obtained from ultrasound-based imaging techniques. Without limitation, the ultrasound-based imaging techniques may include intracardiac echocardiography (ICE), transthoracic echocardiography (TTE), and transesophageal echocardiography (TEE). ICE imaging may provide real-time visualization of cardiac structures from within the heart, enabling precise catheter positioning and monitoring of cardiac ablation procedures. TTE imaging may be utilized for non-invasive assessment of cardiac function, whereas TEE imaging may offer enhanced resolution of posterior cardiac structures by placing an ultrasound probe in the esophagus. The system may integrate these imaging techniques to generate detailed anatomical maps and track procedural progress. In some embodiments, the system may collect image data from computed tomography (CT) imaging to generate high-resolution, three-dimensional representations of anatomical structures. CT imaging may be particularly useful for pre-procedural planning, enabling physicians to visualize the pulmonary veins, left atrial anatomy, or other critical structures with precision. The system may incorporate CT scan data to enhance the accuracy of catheter navigation, lesion placement, and overall procedural strategy. Additionally and or alternatively, contrast-enhanced CT imaging may be used to evaluate vascular integrity and identify potential obstructions or abnormalities. In some embodiments, magnetic resonance imaging (MRI) data may be included as part of the image data collection process. MRI imaging may offer superior soft tissue contrast, enabling the identification of myocardial scarring, fibrosis, or other structural abnormalities that may impact procedural success. The system may utilize MRI-based electroanatomic mapping to assist in targeting regions of interest for ablation procedures. Additionally and or alternatively, functional MRI sequences may be employed to assess myocardial perfusion and cardiac viability, contributing to a comprehensive understanding of the patient's condition. In some embodiments, the system may collect image data from fluoroscopy or X-ray imaging to provide continuous real-time guidance during interventional procedures. Fluoroscopic imaging may be particularly useful in catheter-based interventions, offering dynamic visualization of catheter movement and device deployment. The system may integrate fluoroscopic images with other imaging modalities, such as ICE or electroanatomic mapping, to reduce radiation exposure while maintaining procedural accuracy. Advanced fluoroscopy techniques, including rotational angiography, may be employed to enhance visualization of complex anatomical structures. In some embodiments, the system may utilize hybrid imaging approaches by combining multiple imaging modalities to improve diagnostic accuracy and procedural outcomes. For example, CT-MRI fusion imaging may be employed to combine high-resolution anatomical detail with functional tissue characterization. Similarly, ultrasound-fluoroscopy fusion imaging may enable real-time guidance while minimizing radiation exposure. The system may dynamically process and overlay these imaging datasets to assist clinicians in real-time decision-making, enhance procedural workflow, and improve overall treatment efficacy. In some embodiments, procedure data 136 may include ultrasonic image data. In some embodiments, procedure data 136 may include a Pulsed Field Ablation (PFA) device parameter. In some embodiments, procedure data 136, methods of processing procedure data 136, and/or methods of gathering procedure data 136 may be consistent with U.S. patent application Ser. No. 18/646,991 (having attorney docket number 1518-142USU1), filed on Apr. 26, 2024, and titled “METHOD AND APPARATUS FOR PREDICTING PULSED FIELD ABLATION DURABILITY,” the entirety of which is hereby incorporated by reference. In a non-limiting example, procedure data 136 may include heart rate data of a subject from a heart rate sensor. Procedure data 136 may include, in additional non-limiting examples, ultrasonic image data, echocardiogram data, intracardiac echo (ICE) image data, transthoracic echocardiogram (TTE) data, transesophageal echocardiogram (TEE) data, and point of care ultrasound (POCUS) data. Procedure data 136 may include, in additional non-limiting examples, data gathered by a catheter configured for ablation, mapping, and/or ICE imaging. In some embodiments, system 100 may include an ultrasound device configured to generate ultrasound data. In some embodiments, system may include an ICE catheter configured to detect ICE data.

With continued reference to FIG. 1, the image data may include real-time image data. As used in this disclosure, “real-time image data” is image data that is continuously acquired, processed, and displayed with minimal delay. In an embodiment, the real-time image data may enable quick visualizations and analysis of dynamic physiological structures or procedural environments. Real-time image data may be captured using imaging modalities such as ultrasound, fluoroscopy, or intraoperative MRI, providing instant feedback to guide medical procedures, monitor organ function, or assess anatomical changes in response to interventions. In an embodiment, the real-time image data may include intracardiac echocardiography real-time image data. In an embodiment, the ICE real-time image data may provide continuous high-resolution images from within the heart, allowing clinicians to monitor catheter positioning, assess lesion formation during pulsed field ablation (PFA), and detect procedural complications such as pericardial effusion in real-time. In an embodiment, the real-time image data may include fluoroscopic real-time image data. In an embodiment, the fluoroscopic real-time image data may generate a continuous X-ray image of the target anatomy, enabling clinicians to visualize guidewire navigation, confirm the deployment of medical devices, and assess vascular flow dynamics as the procedure progresses. The system may overlay fluoroscopic images with electroanatomic mapping data to enhance precision while reducing radiation exposure. In an embodiment, the real-time image data may include intraoperative MRI real-time image data. In an embodiment, the intraoperative MRI real-time image data may provide real-time visualization of soft tissue structures during neurosurgical or oncological procedures. The real-time image data may enable continuous imaging updates, allowing surgeons to confirm tumor resection margins, monitor brain shift during surgery, and adjust surgical plans dynamically based on real-time anatomical feedback. In an embodiment, the real-time image data may include point-of-care ultrasound (POCUS) real-time image data. In an embodiment, the POCUS real-time image data may provide near immediate visualization of the heart, lungs, or abdomen to assess conditions such as cardiac tamponade, pneumothorax, or internal bleeding. The system may integrate POCUS real-time image data with artificial intelligence algorithms to assist in automated interpretation and clinical decision-making. In an embodiment, the real-time image data may include endoscopic real-time image data. In an embodiment, the endoscopic real-time image data may enable direct visualization of internal organ surfaces during gastrointestinal or bronchoscopic procedures. The real-time image data may assist in detecting ulcers, polyps, or tumors while guiding biopsies or therapeutic interventions such as laser ablation or stent placement. The system may process endoscopic image data to enhance contrast, detect anomalies, or provide augmented reality overlays for improved procedural accuracy. In an embodiment, the real-time image data may include Doppler ultrasound real-time image data. In an embodiment, the Doppler ultrasound real-time image data may assess blood flow characteristics within arteries and veins in real time. The real-time image data may enable clinicians to evaluate vascular occlusions, monitor blood velocity changes, and assess cardiac output dynamically. The system may integrate Doppler ultrasound data with AI-driven hemodynamic models to provide real-time alerts for abnormal flow patterns, assisting in early detection of conditions such as deep vein thrombosis or carotid artery stenosis.

With continued reference to FIG. 1, the image data may include pre-recorded data. As used in this disclosure, “pre-recorded data” is image data that has been previously acquired, stored, and retrieved for later analysis. The pre-recorded data may include historical imaging records, diagnostic scans, procedural imaging datasets, and the like. In an embodiment, the pre-recorded data may be used to compare with real-time imaging, assist in treatment planning, enhance clinical decision-making, and the like. The system may utilize pre-recorded data from various sources, including electronic health records, prior imaging studies, or research databases, to provide comprehensive insights into a subject's medical history and condition. In an embodiment, the pre-recorded data may include electronic health record image data. In an embodiment, the EHR image data may include stored imaging records from previous diagnostic procedures such as MRI, CT, X-ray, or ultrasound scans. The system may retrieve this data to compare historical imaging findings with real-time scans, enabling clinicians to assess disease progression, evaluate treatment response, or identify anatomical changes over time. In an embodiment, the pre-recorded data may include prior diagnostic imaging data. In an embodiment, the prior diagnostic imaging data may consist of previously acquired medical images, such as CT angiography scans used to assess coronary artery disease or MRI scans used to detect neurological abnormalities. The system may integrate this pre-recorded data with current imaging to provide a longitudinal view of the subject's condition, aiding in the diagnosis and management of chronic diseases. In an embodiment, the pre-recorded data may include pre-procedural planning image data. In an embodiment, the pre-procedural planning image data may include 3D reconstructions of cardiac anatomy, vascular maps, segmented organ models, and the like generated from prior CT or MRI scans. The system may overlay these images with real-time fluoroscopic or ultrasound data to enhance procedural accuracy during interventions such as catheter-based ablations or stent placements. In an embodiment, the pre-recorded data may include historical ultrasound image data. In an embodiment, the historical ultrasound image data may consist of echocardiographic recordings, Doppler flow studies, or fetal ultrasound scans stored for retrospective analysis. The system may compare pre-recorded ultrasound data with live imaging to evaluate structural heart changes, monitor fetal development, or detect anomalies that were previously unrecognized. In an embodiment, the pre-recorded data may include archived surgical or interventional imaging data. In an embodiment, the archived surgical or interventional imaging data may include intraoperative fluoroscopic recordings, preoperative angiograms, or robotic-assisted surgical imaging stored for training, quality assurance, or procedural review. The system may retrieve and analyze this data to refine surgical techniques, enhance operator learning, or facilitate retrospective outcome assessments. In an embodiment, the pre-recorded data may include AI-enhanced historical imaging datasets. In an embodiment, the Al-enhanced historical imaging datasets may be pre-processed imaging records that have been analyzed using artificial intelligence algorithms to highlight regions of interest, detect pathology, or predict clinical outcomes. The system may utilize these datasets to assist in automated diagnostics, optimize treatment planning, or improve predictive analytics in personalized medicine.

Still referring to FIG. 1, in some embodiments, system 100 may receive electronic health record data 140 from electronic health record database 148. As used herein, “electronic health record data” is digital data describing a health state of a subject, digital data describing a historical health state of a subject, digital data describing a historical medical event of a subject, or a combination thereof. Electronic health record data may include, in non-limiting examples, data describing a medical procedure performed on a subject, data describing a symptom of a medical condition of a subject, and results of a medical test performed on a subject.

Still referring to FIG. 1, in some embodiments, system 100 may determine, using agent orchestrator 156, an agent selection datum. As used herein, an “agent orchestrator” is a system for activating one or more agents. As used herein, an “agent selection datum” is a datum identifying an agent to be activated. As used herein, an “agent” is a computer program which acts at the instruction of another computer program. In some embodiments, system 100 may determine an agent selection datum based on a user input. In some embodiments, system 100 may determine an agent selection datum using an agent selection machine learning model. Agent selection machine learning model may be trained using a supervised learning algorithm. Agent selection machine learning model may include a classifier. Agent selection machine learning model may include a neural network. Agent selection machine learning model may be trained on a training dataset including example user inputs, associated with example agent selection data. Such a training dataset may be obtained by, for example, manually assigning prompts to the appropriate agents. In some embodiments, a pre-trained model may be used, such as a pre-trained language model. In some embodiments, a pre-trained model may be fine-tuned to a task of assigning prompts to agents. Once agent selection machine learning model is trained, it may be used to determine an agent selection datum. System 100 may input a user input into agent selection machine learning model, and system 100 may receive an agent selection datum from the model.

Still referring to FIG. 1, system 100 determines, using the agent orchestrator, the first agent selection datum and a fallback protocol, wherein determining the first agent selection datum comprises generating the first agent selection datum as a function of the first user input using a trained agent selection machine learning model, using the first agent corresponding to the first agent selection datum, determine a first agent output, by inputting into the first agent the procedure data and receiving, as an output from the first agent, the first agent output. As used in this disclosure, a “trained agent selection machine learning model” is a machine learning-based model configured to determine an agent selection datum. In an embodiment, the trained agent selection machine learning model may determine an agent selection datum based on user input and procedural data. The trained agent selection machine learning model may be trained on a diverse dataset comprising historical interactions, agent performance metrics, contextual parameters, and user preferences to enable accurate selection of an appropriate agent for a given task. The training data for the trained agent selection machine learning model may include, without limitation, labeled examples of agent selections, success rates of different agents in varying conditions, and reinforcement signals indicating the effectiveness of prior agent choices. The trained agent selection machine learning model may utilize supervised learning, reinforcement learning, or a combination of both to refine its decision-making process as described herein. In some embodiments, the trained agent selection machine learning model may generate a fallback protocol, as described in more detail below, to handle scenarios where the initially selected agent is unable to provide a satisfactory response. The fallback protocol may be dynamically generated based on user input, task complexity, agent availability, and/or system confidence levels. For example, without limitation, if the first agent selection datum indicates an agent that fails to produce a valid output, the fallback protocol may trigger an alternative agent selection, escalate the request to a human supervisor, or modify the procedural data to better align with the agent's capabilities. The fallback protocol may differ depending on the nature of the user request, ensuring adaptive and context-aware handling of agent failures. When the system 100 determines the first agent selection datum, it may utilize the trained agent selection machine learning model to analyze the first user input and generate a selection that best aligns with the expected outcome. The selected agent may then process the procedure data to generate a first agent output. If the system 100 detects an issue with the first agent output, such as low confidence, incomplete responses, or errors, the fallback protocol may be activated to ensure continuity and reliability of the response. Continuing, this approach may enhance the robustness of agent-based workflows by leveraging machine learning to optimize agent selection while incorporating intelligent fallback mechanisms for improved adaptability.

With continued reference to FIG. 1, the at least a processor may be further configured to implement a fallback protocol for the agent orchestrator, wherein the fallback protocol may include determining whether electronic health record data corresponding to the first user input is available, in response to determining that the electronic health record data is available, selecting an electronic health record-based agent to process the first agent selection datum and generate the first agent output, and in response to determining that the electronic health record data is unavailable, selecting an inference agent to process the first agent selection datum using one or more algorithms trained to operate on electrocardiogram data. As used in this disclosure, a “fallback protocol” is a decision-making process implemented by the agent orchestrator that selects an alternative data source when a primary resource is unavailable. In an embodiment, the fallback protocol ensures continuous system functionality and accurate response generation. As used in this disclosure, an “electronic health record-based agent” is a software component within the agent orchestrator that processes user input and generates a response. In an embodiment, the electronic health record-based agent may use electronic health record data, prioritizing historical and structured medical information for decision-making. As used in this disclosure, an “inference agent” is a computational model that processes user input and generates an output by analyzing real-time or previously collected data. In an embodiment, the inference agent may use machine learning algorithms, probabilistic models, rule-based logic, and the like. As used in this disclosure, “electrocardiogram data” is physiological data obtained from an electrocardiogram. In an embodiment, the electrocardiogram data may measure electrical activity in the heart and may be used to analyze cardiac rhythms, detect abnormalities, and infer cardiovascular conditions.

With continued reference to FIG. 1, the at least a processor is further configured to determine whether electronic health record data is available by querying connected databases and verifying access, conditionally select the electronic health record-based agent to retrieve and process structured patient data if available, conditionally activate the inference agent to analyze real-time electrocardiogram data if the electronic health record data is unavailable. As used in this disclosure, “connected databases” are structured data repositories that store, manage, and provide access to electronic health record (EHR) data, real-time physiological data, and historical medical records through system queries. The connected databases may be locally stored, cloud-based, or part of an integrated hospital or healthcare network. In some embodiments, the connected databases may include various structured data repositories that store, manage, and provide access to electronic health record (EHR) data, medical imaging, and real-time physiological data. These databases may be locally stored within a healthcare institution, hosted in a cloud-based infrastructure, or integrated within a broader health information exchange (HIE) network. The system may dynamically query these databases to determine data availability and select the most appropriate agent for processing the request. In some embodiments, the connected databases may include electronic health record (EHR) systems, which store structured patient information such as medical history, physician notes, laboratory results, and past procedures. The system may access EHR platforms such as Epic, Cerner, or Meditech, retrieving relevant patient data for analysis. The EHR data may be used to compare historical trends, confirm past diagnoses, or provide continuity in patient care. In some embodiments, the connected databases may include medical imaging repositories, which store and manage diagnostic imaging data. The system may access Picture Archiving and Communication Systems (PACS) or DICOM-compliant storage solutions to retrieve MRI, CT, ultrasound, and X-ray images for review. The imaging data may be analyzed to track anatomical changes, assist in pre-procedural planning, or support real-time guidance during interventions. In some embodiments, the connected databases may include cardiac monitoring databases, which store electrocardiogram (ECG) data, Holter monitor recordings, and telemetry from wearable or implanted cardiac devices. The system may retrieve data from platforms such as MUSE ECG Management System or CardioPACs to analyze historical ECG patterns, assess arrhythmias, or generate predictive insights related to cardiovascular conditions. In some embodiments, the connected databases may include cloud-based health information exchanges (HIEs), which facilitate interoperability between different healthcare providers. The system may access networks such as CommonWell Health Alliance or Carequality to retrieve patient records across institutions. The HIE data may be used to enhance care coordination, prevent redundant testing, and provide comprehensive patient insights regardless of where the patient was previously treated. In some embodiments, the connected databases may include AI-driven predictive analytics databases, which store and analyze patient health data using machine learning models. These databases may integrate structured EHR data with real-time physiological monitoring to generate predictive insights for clinical decision support. The system may leverage AI-enhanced datasets to detect early signs of disease progression, recommend personalized treatment plans, or provide automated risk assessments. The system may initiate this process upon receiving a user input requiring patient-specific data. If the EHR data is available, the processor May conditionally select the electronic health record-based agent, which retrieves and processes structured patient data to generate an appropriate response. If the EHR data is unavailable, the processor may conditionally activate an inference agent, which analyzes real-time electrocardiogram (ECG) data to generate a diagnostic or procedural output. This fallback mechanism ensures that the system remains functional, regardless of data availability, allowing real-time decision-making based on the best available information.

Still referring to FIG. 1, in some embodiments, agent selection machine learning model may include a language model. In some embodiments, inputs to agent selection machine learning model may include user input 128. In some embodiments, user input 128 may be transcribed using an automatic speech recognition system as described above before being input into agent selection machine learning model. In some embodiments, a language model may include a large language model. In some embodiments, a language model may include a pre-trained language model, such as a language model trained on a corpus of information not specific to applications for selecting agents. In some embodiments, a language model may include a neural network. In some embodiments, a pre-trained language model may be fine-tuned for a specific purpose, such as selection of agents. In some embodiments, low-rank adaptation may be used to fine-tune a neural network.

Still referring to FIG. 1, in some embodiments, a language model may be used to process user input. As used herein, a “language model” is a program capable of interpreting natural language, generating natural language, or both. In some embodiments, a language model may be configured to interpret the output of an automatic speech recognition function and/or an OCR function. A language model may include a neural network. A language model may be trained using a dataset that includes natural language.

Still referring to FIG. 1, in some embodiments, a language model may be configured to extract one or more words from a document. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols. Textual data may be parsed into tokens, which may include a simple word (sequence of letters separated by whitespace) or more generally a sequence of characters. As used herein, a “token,” is a smaller, individual grouping of text from a larger source of text. Tokens may be broken up by word, pair of words, sentence, or other delimitations. Tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as chains, for example for use as a Markov chain or Hidden Markov Model.

Still referring to FIG. 1, generating language model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, processor 104 may determine one or more language elements in user input by identifying and/or detecting associations between one or more language elements (including phonemes or phonological elements, morphemes or morphological elements, syntax or syntactic elements, semantics or semantic elements, and pragmatic elements) extracted from at least user input, including without limitation mathematical associations, between such words. Associations between language elements and relationships of such categories to other such term may include, without limitation, mathematical associations, including without limitation statistical correlations between any language element and any other language element and/or Language elements. Processor 104 may compare an input such as a sentence from user input with a list of keywords or a dictionary to identify language elements. For example, processor 104 may identify whitespace and punctuation in a sentence and extract elements comprising a string of letters, numbers or characters occurring adjacent to the whitespace and punctuation. Processor 104 may then compare each of these with a list of keywords or a dictionary. Based on the determined keywords or meanings associated with each of the strings, processor 104 may determine an association between one or more of the extracted strings and a feature of a user input and/or a medical procedure, such as an association between the phrase “QRS complex” and an electrocardiogram. Associations may take the form of statistical correlations and/or mathematical associations, which may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory.

Still referring to FIG. 1, processor 104 may be configured to determine one or more language elements in user input using machine learning. For example, processor 104 may generate the language processing model by any suitable method, including without limitation a natural language processing classification algorithm; language processing model may include a natural language process classification model that enumerates and/or derives statistical relationships between input terms and output terms. An algorithm to generate language processing model may include a stochastic gradient descent algorithm, which may include a method that iteratively optimizes an objective function, such as an objective function representing a statistical estimation of relationships between terms, including relationships between input language elements and output patterns or conversational styles in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between an extracted word, phrase, and/or other semantic unit. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Still referring to FIG. 1, processor 104 may be configured to determine one or more language elements in user input using machine learning by first creating or receiving language classification training data. Training data may include data containing correlations that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below.

Still referring to FIG. 1, language classification training data may be a training data set containing associations between language element inputs and associated language element outputs. Language element inputs and outputs may be categorized by communication form such as written language elements, spoken language elements, typed language elements, or language elements communicated in any suitable manner. Language elements may be categorized by component type, such as phonemes or phonological elements, morphemes or morphological elements, syntax or syntactic elements, semantics or semantic elements, and pragmatic elements. Associations may be made between similar communication types of language elements (e.g. associating one written language element with another written language element) or different language elements (e.g. associating a spoken language element with a written representation of the same language element). Associations may be identified between similar communication types of two different language elements, for example written input consisting of the syntactic element “that” may be associated with written phonemes/th/,/a/, and/t/. Associations may be identified between different communication forms of different language elements. For example, the spoken form of the syntactic element “that” and the associated written phonemes above. Language classification training data may be created using a classifier such as a language classifier. An exemplary classifier may be created, instantiated, and/or run using processor 104, or another computing device. Language classification training data may create associations between any type of language element in any format and other type of language element in any format. Additionally, or alternatively, language classification training data may associate language element input data to a feature related to a user input and/or a medical procedure. For example, language classification training data may associate occurrences of the syntactic elements “where,” and “catheter,” in a single sentence with a request for data describing a position of a cardiac catheter.

Still referring to FIG. 1, processor 104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

Still referring to FIG. 1, processor 104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

Still referring to FIG. 1, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm:

$$l = \sqrt{{Σ}_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, language processing module may use a corpus of documents to generate associations between language elements in a language processing module, and a diagnostic engine may then use such associations to analyze words extracted from one or more documents and determine that the one or more documents indicate significance of a category. In an embodiment, a computing device may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into a computing device. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

Still referring to FIG. 1, in some embodiments, a language model may include a large language model (LLM). A “large language model,” as used herein, is a deep learning data structure that can recognize, summarize, translate, predict and/or generate text and other content based on knowledge gained from massive datasets. Large language models may be trained on large sets of data. Training sets may be drawn from diverse sets of data such as, as non-limiting examples, novels, blog posts, articles, emails, unstructured data, electronic records, and the like. In some embodiments, training sets may include a variety of subject matters, such as, as nonlimiting examples, medical report documents, electronic health records, entity documents, business documents, inventory documentation, emails, user communications, advertising documents, newspaper articles, and the like. In some embodiments, training sets of an LLM may include information from one or more public or private databases. As a non-limiting example, training sets may include databases associated with an entity. In some embodiments, training sets may include portions of documents associated with the electronic records correlated to examples of outputs. In an embodiment, an LLM may include one or more architectures based on capability requirements of an LLM. Exemplary architectures may include, without limitation, GPT (Generative Pretrained Transformer), BERT (Bidirectional Encoder Representations from Transformers), T5 (Text-To-Text Transfer Transformer), and the like. Architecture choice may depend on a needed capability such generative, contextual, or other specific capabilities.

With continued reference to FIG. 1, in some embodiments, an LLM may be generally trained. As used in this disclosure, a “generally trained” LLM is an LLM that is trained on a general training set comprising a variety of subject matters, data sets, and fields. In some embodiments, an LLM may be initially generally trained. Additionally, or alternatively, an LLM may be specifically trained. As used in this disclosure, a “specifically trained” LLM is an LLM that is trained on a specific training set, wherein the specific training set includes data including specific correlations for the LLM to learn. As a non-limiting example, an LLM may be generally trained on a general training set, then specifically trained on a specific training set. In an embodiment, specific training of an LLM may be performed using a supervised machine learning process. In some embodiments, generally training an LLM may be performed using an unsupervised machine learning process. As a non-limiting example, specific training set may include information from a database. As a non-limiting example, specific training set may include text related to the users such as user specific data for electronic records correlated to examples of outputs. In an embodiment, training one or more machine learning models may include setting the parameters of the one or more models (weights and biases) either randomly or using a pretrained model. Generally training one or more machine learning models on a large corpus of text data can provide a starting point for fine-tuning on a specific task. A model such as an LLM may learn by adjusting its parameters during the training process to minimize a defined loss function, which measures the difference between predicted outputs and ground truth. Once a model has been generally trained, the model may then be specifically trained to fine-tune the pretrained model on task-specific data to adapt it to the target task. Fine-tuning may involve training a model with task-specific training data, adjusting the model's weights to optimize performance for the particular task. In some cases, this may include optimizing the model's performance by fine-tuning hyperparameters such as learning rate, batch size, and regularization. Hyperparameter tuning may help in achieving the best performance and convergence during training.

Still referring to FIG. 1, in some embodiments, a pre-trained neural network may be fine-tuned. In some embodiments, a fine-tuning process may include freezing a pre-trained weight matrix (W₀) of a layer of a pre-trained model and determining an accumulated gradient update (AW) of the layer during adaptation of the pre-trained weight matrix. W₀ may be a matrix with W₀∈ [symbol] ^{d×k}. ΔW may be a matrix with the same dimensions as W₀. When running the neural network, a forward pass (h) of a layer may be determined using the formula h=W₀X+ΔWX where X is the input from a previous layer. In some embodiments, a plurality of layers of a neural network may be fine-tuned. Fine-tuning a pre-trained neural network may improve efficiency of neural network training. In a non-limiting example, a neural network trained on a broad variety of data may be fine-tuned for a specific purpose.

Still referring to FIG. 1, in some embodiments, a pre-trained neural network may be fine-tuned using low rank adaptation. In low rank adaptation, ΔW is replaced by low rank decomposition matrices A and B, using the formula ΔW=BA. B and A may be matrices with B∈ [symbol] ^{d×r}, and A∈ [symbol] ^{r×k}. Hyperparameter r may represent the rank of a low rank adaptation module and may be chosen such that r<min(d,k) based on factors described below. A forward pass of a layer trained using low rank adaptation may have the formula h=W₀X+BAX. A random Gaussian initialization may be used to determine initial values for A and initial values of B may be set to 0, such that ΔW=BA is 0 before training. ΔWX may be scaled by α/r during training, where α is a constant in r. In some embodiments, α may be tuned as one would tune a learning rate. In some embodiments, α may be set and not tuned further. In some embodiments, a plurality of layers of a neural network may be fine-tuned using low rank adaptation. Fine-tuning a pre-trained neural network using low-rank adaptation may reduce memory and/or processing power requirements of fine-tuning the neural network, as B and A have fewer trainable parameters than ΔW would have in a non-low rank adaptation approach. In some embodiments, this difference may lead to substantial improvements where ΔW has very large dimensions. The value of hyperparameter r may influence the degree to which low rank adaptation reduces memory and/or processing power requirements. In some embodiments, setting r too low may result in information loss. In some embodiments, setting r too high may result in increased memory and processing power usage for fine-tuning the neural network relative to a lower r. In some embodiments, r may be a number of linearly independent rows or columns of ΔW.

With continued reference to FIG. 1, in some embodiments an LLM may include and/or be produced using Generative Pretrained Transformer (GPT), GPT-2, GPT-3, GPT-4, and the like. GPT, GPT-2, GPT-3, GPT-3.5, and GPT-4 are products of Open AI Inc., of San Francisco, CA. An LLM may include a text prediction based algorithm configured to receive an article and apply a probability distribution to the words already typed in a sentence to work out the most likely word to come next in augmented articles. For example, if some words that have already been typed are “Nice to meet”, then it may be highly likely that the word “you” will come next. An LLM may output such predictions by ranking words by likelihood or a prompt parameter. For the example given above, an LLM may score “you” as the most likely, “your” as the next most likely, “his” or “her” next, and the like. An LLM may include an encoder component and a decoder component.

Still referring to FIG. 1, an LLM may include a transformer architecture. In some embodiments, encoder component of an LLM may include transformer architecture. A “transformer architecture,” for the purposes of this disclosure is a neural network architecture that uses self-attention and positional encoding. Transformer architecture may be designed to process sequential input data, such as natural language, with applications towards tasks such as translation and text summarization. Transformer architecture may process the entire input all at once. “Positional encoding,” for the purposes of this disclosure, refers to a data processing technique that encodes the location or position of an entity in a sequence. In some embodiments, each position in the sequence may be assigned a unique representation. In some embodiments, positional encoding may include mapping each position in the sequence to a position vector. In some embodiments, trigonometric functions, such as sine and cosine, may be used to determine the values in the position vector. In some embodiments, position vectors for a plurality of positions in a sequence may be assembled into a position matrix, wherein each row of position matrix may represent a position in the sequence.

With continued reference to FIG. 1, an LLM and/or transformer architecture may include an attention mechanism. An “attention mechanism,” as used herein, is a part of a neural architecture that enables a system to dynamically quantify the relevant features of the input data. In the case of natural language processing, input data may be a sequence of textual elements. It may be applied directly to the raw input or to its higher-level representation.

With continued reference to FIG. 1, attention mechanism may represent an improvement over a limitation of an encoder-decoder model. An encoder-decider model encodes an input sequence to one fixed length vector from which the output is decoded at each time step. This issue may be seen as a problem when decoding long sequences because it may make it difficult for the neural network to cope with long sentences, such as those that are longer than the sentences in the training corpus. Applying an attention mechanism, an LLM may predict the next word by searching for a set of positions in a source sentence where the most relevant information is concentrated. An LLM may then predict the next word based on context vectors associated with these source positions and all the previously generated target words, such as textual data of a dictionary correlated to a prompt in a training data set. A “context vector,” as used herein, are fixed-length vector representations useful for document retrieval and word sense disambiguation.

Still referring to FIG. 1, attention mechanism may include, without limitation, generalized attention self-attention, multi-head attention, additive attention, global attention, and the like. In generalized attention, when a sequence of words or an image is fed to an LLM, it may verify each element of the input sequence and compare it against the output sequence. Each iteration may involve the mechanism's encoder capturing the input sequence and comparing it with each element of the decoder's sequence. From the comparison scores, the mechanism may then select the words or parts of the image that it needs to pay attention to. In self-attention, an LLM may pick up particular parts at different positions in the input sequence and over time compute an initial composition of the output sequence. In multi-head attention, an LLM may include a transformer model of an attention mechanism. Attention mechanisms, as described above, may provide context for any position in the input sequence. For example, if the input data is a natural language sentence, the transformer does not have to process one word at a time. In multi-head attention, computations by an LLM may be repeated over several iterations, each computation may form parallel layers known as attention heads. Each separate head may independently pass the input sequence and corresponding output sequence element through a separate head. A final attention score may be produced by combining attention scores at each head so that every nuance of the input sequence is taken into consideration. In additive attention (Bahdanau attention mechanism), an LLM may make use of attention alignment scores based on a number of factors. Alignment scores may be calculated at different points in a neural network, and/or at different stages represented by discrete neural networks. Source or input sequence words are correlated with target or output sequence words but not to an exact degree. This correlation may take into account all hidden states and the final alignment score is the summation of the matrix of alignment scores. In global attention (Luong mechanism), in situations where neural machine translations are required, an LLM may either attend to all source words or predict the target sentence, thereby attending to a smaller subset of words.

With continued reference to FIG. 1, multi-headed attention in encoder may apply a specific attention mechanism called self-attention. Self-attention allows models such as an LLM or components thereof to associate each word in the input, to other words. As a non-limiting example, an LLM may learn to associate the word “you”, with “how” and “are”. It's also possible that an LLM learns that words structured in this pattern are typically a question and to respond appropriately. In some embodiments, to achieve self-attention, input may be fed into three distinct fully connected neural network layers to create query, key, and value vectors. A query vector may include an entity's learned representation for comparison to determine attention score. A key vector may include an entity's learned representation for determining the entity's relevance and attention weight. A value vector may include data used to generate output representations. Query, key, and value vectors may be fed through a linear layer; then, the query and key vectors may be multiplied using dot product matrix multiplication in order to produce a score matrix. The score matrix may determine the amount of focus for a word should be put on other words (thus, each word may be a score that corresponds to other words in the time-step). The values in score matrix may be scaled down. As a non-limiting example, score matrix may be divided by the square root of the dimension of the query and key vectors. In some embodiments, the softmax of the scaled scores in score matrix may be taken. The output of this softmax function may be called the attention weights. Attention weights may be multiplied by your value vector to obtain an output vector. The output vector may then be fed through a final linear layer.

Still referencing FIG. 1, in order to use self-attention in a multi-headed attention computation, query, key, and value may be split into N vectors before applying self-attention. Each self-attention process may be called a “head.” Each head may produce an output vector and each output vector from each head may be concatenated into a single vector. This single vector may then be fed through the final linear layer discussed above. In theory, each head can learn something different from the input, therefore giving the encoder model more representation power.

With continued reference to FIG. 1, encoder of transformer may include a residual connection. Residual connection may include adding the output from multi-headed attention to the positional input embedding. In some embodiments, the output from residual connection may go through a layer normalization. In some embodiments, the normalized residual output may be projected through a pointwise feed-forward network for further processing. The pointwise feed-forward network may include a couple of linear layers with a ReLU activation in between. The output may then be added to the input of the pointwise feed-forward network and further normalized.

Continuing to refer to FIG. 1, transformer architecture may include a decoder. Decoder may a multi-headed attention layer, a pointwise feed-forward layer, one or more residual connections, and layer normalization (particularly after each sub-layer), as discussed in more detail above. In some embodiments, decoder may include two multi-headed attention layers. In some embodiments, decoder may be autoregressive. For the purposes of this disclosure, “autoregressive” means that the decoder takes in a list of previous outputs as inputs along with encoder outputs containing attention information from the input.

With further reference to FIG. 1, in some embodiments, input to decoder may go through an embedding layer and positional encoding layer in order to obtain positional embeddings. Decoder may include a first multi-headed attention layer, wherein the first multi-headed attention layer may receive positional embeddings.

With continued reference to FIG. 1, first multi-headed attention layer may be configured to not condition to future tokens. As a non-limiting example, when computing attention scores on the word “am,” decoder should not have access to the word “fine” in “I am fine,” because that word is a future word that was generated after. The word “am” should only have access to itself and the words before it. In some embodiments, this may be accomplished by implementing a look-ahead mask. Look ahead mask is a matrix of the same dimensions as the scaled attention score matrix that is filled with “0s” and negative infinities. For example, the top right triangle portion of look-ahead mask may be filled with negative infinities. Look-ahead mask may be added to scaled attention score matrix to obtain a masked score matrix. Masked score matrix may include scaled attention scores in the lower-left triangle of the matrix and negative infinities in the upper-right triangle of the matrix. Then, when the softmax of this matrix is taken, the negative infinities will be zeroed out; this leaves zero attention scores for “future tokens.”

Still referring to FIG. 1, second multi-headed attention layer may use encoder outputs as queries and keys and the outputs from the first multi-headed attention layer as values. This process matches the encoder's input to the decoder's input, allowing the decoder to decide which encoder input is relevant to put a focus on. The output from second multi-headed attention layer may be fed through a pointwise feedforward layer for further processing.

With continued reference to FIG. 1, the output of the pointwise feedforward layer may be fed through a final linear layer. This final linear layer may act as a classifier. This classifier may be as big as the number of classes that you have. For example, if you have 10,000 classes for 10,000 words, the output of that classifier will be of size 10,000. The output of this classifier may be fed into a softmax layer which may serve to produce probability scores between zero and one. The index may be taken of the highest probability score in order to determine a predicted word.

Still referring to FIG. 1, decoder may take this output and add it to the decoder inputs. Decoder may continue decoding until a token is predicted. Decoder may stop decoding once it predicts an end token.

Continuing to refer to FIG. 1, in some embodiment, decoder may be stacked N layers high, with each layer taking in inputs from the encoder and layers before it. Stacking layers may allow an LLM to learn to extract and focus on different combinations of attention from its attention heads.

With continued reference to FIG. 1, an LLM may receive an input. Input may include a string of one or more characters. Inputs may additionally include unstructured data. For example, input may include one or more words, a sentence, a paragraph, a thought, a query, and the like. A “query” for the purposes of the disclosure is a string of characters that poses a question. In some embodiments, an input may include user input 128.

With continued reference to FIG. 1, an LLM may generate at least one annotation as an output. At least one annotation may be any annotation as described herein. In some embodiments, an LLM may include multiple sets of transformer architecture as described above. Output may include a textual output. A “textual output,” for the purposes of this disclosure is an output comprising a string of one or more characters. Textual output may include, for example, a plurality of annotations for unstructured data. In some embodiments, textual output may include a phrase or sentence identifying the status of a user query. In some embodiments, textual output may include a sentence or plurality of sentences describing a response to a user query. As a non-limiting example, this may include restrictions, timing, advice, dangers, benefits, and the like.

Still referring to FIG. 1, in some embodiments, system 100 may generate encoded user input 160. As used herein, “encoded user input” is a mathematical representation of user input generated based on relationships between components of the user input. In some embodiments, encoded user input 160 may be generated using a transformer model. In some embodiments, a language model, such as first language model 164, may include a transformer model, may generate an encoded user input, and/or may receive as an input an encoded user input. In some embodiments, generating an agent selection datum as a function of a user input may include inputting into a trained agent selection machine learning model encoded user input 160 and receiving, as an output from the agent selection machine learning model, the first agent selection datum.

Still referring to FIG. 1, in some embodiments, system 100 may generate agent selection datum without first receiving a user input. For example, system 100 may passively monitor a medical procedure and may generate an agent selection datum as a function of data received through such monitoring. Passive monitoring may include, in non-limiting examples, receipt of procedure data 136 and/or electronic health record data 140. For example, system 100 may monitor live ECG data and/or other live procedure data for abnormalities. In some embodiments, a first subset of agents may be used while system 100 is passively monitoring a procedure and a second subset of agents may be activated based on user input 128. In some embodiments, system 100 may determine an agent selection datum during passive monitoring. In some embodiments, determination of an agent selection datum during passive monitoring may include use of agent selection machine learning model. In this instance, an input into agent selection machine learning model may include procedure data such as ECG data, and agent selection machine learning model may output agent selection datum. Such agent selection machine learning model may be trained on a training dataset including example procedure data associated with example agent selection data. In some embodiments, agent orchestrator may match the live procedure data to the corresponding monitoring agent. For example, if live procedure data includes live ECG data, agent orchestrator may determine an agent selection datum corresponding to an ECG monitoring agent. For example, if live procedure data includes live ablation data, agent orchestrator may determine an agent selection datum corresponding to an ablation monitoring agent. The matching of live procedure data to various agents may, in some embodiments, not require the use of machine-learning.

Still referring to FIG. 1, in some embodiments, system 100 may retrain agent selection machine learning model. In some embodiments, system 100 may receive additional training data and may incorporate such training data into a dataset used to retrain agent selection machine learning model. Such dataset may in some embodiments include data of a prior dataset used to train agent selection machine learning model. In some embodiments, such additional training data may be generated based on user feedback. For example, system 100 may generate an agent selection datum and ultimately an output based on a first user input, may receive a second user input indicating a degree to which such output is responsive to the first user input, and system 100 may generate an element of training data as a function of the first user input, the second user input, and/or the output. For example, if second user input indicates that the output was not responsive to the first user input, then a training datum may be generated which may be used to train agent selection machine learning model such that it is less likely to select the same agent when faced with similar user inputs to first user input. In some embodiments, agent selection machine learning model may be trained using reinforcement learning.

Still referring to FIG. 1, in some embodiments, system 100 may, using an agent of plurality of agents 172 which corresponds to an agent selection datum, determine an agent output 168. As used herein, an “agent output” is data generated by an agent. An agent output may include, in non-limiting examples, a 3D model of a structure such as a heart and a Pulsed Field Ablation (PFA) durability datum. In some embodiments, system 100 may input into an agent procedure data 136 and/or at least a portion of electronic health record data 140 and receive, as an output from the agent, agent output 168.

Still referring to FIG. 1, in some embodiments, system 100 may include a plurality of agents and/or may receive multiple agent outputs. In some embodiments, an agent may produce multiple agent outputs based on the same or different user inputs. In some embodiments, different agents may produce different agent responses based on the same or different user inputs. In a non-limiting example, system 100 may generate a first agent selection datum and a first agent output based on a first user input and/or procedure data 136. In this example, system 100 may further receive, from user interface 124, a second user input, receive, from electronic health record database 148, electronic health record data 140, determine, using agent orchestrator 156, a second agent selection datum by generating the second agent selection datum as a function of the second user input using trained agent selection machine learning model, using a second agent corresponding to the second agent selection datum, determine a second agent output by inputting into the second agent the electronic health record data, receive, as an output from the second agent, the second agent output, and display, using the user interface, the second agent output. Such a process may be used to, for example, display information within an electronic health record of a subject as well as display information about an ongoing procedure. In additional examples, a process involving multiple agents may include an agent described below.

Still referring to FIG. 1, in some embodiments, an agent may be used to generate a 3D model of a structure such as a cardiac structure. In some embodiments, an agent may generate a set of shape parameters representing a structure's shape as a function of ultrasonic image data and a shape identification model trained on a training dataset comprising historical ultrasonic images correlated with historical computed tomography scan data and generate a 3D model of the structure based on the set of shape parameters. Agent output 168 may include such 3D model. In some embodiments, such a process may be consistent with U.S. patent application Ser. No. 18/395,087 (having attorney docket number 1518-110USU1), filed on Dec. 22, 2023, and titled “APPARATUS AND METHOD FOR GENERATING A THREE-DIMENSIONAL (3D) MODEL OF CARDIAC ANATOMY WITH AN OVERLAY,” the entirety of which is hereby incorporated by reference.

With continued reference to FIG. 1, an agent may be configured to receive a set of images of a structure of a subject. As used in this disclosure, a “set of images” refers to a group of one or more visual representations. Set of images may include, without limitation, a two-dimensional image. In some embodiments, set of images may include an ultrasonic image. As used herein, an “ultrasonic image” is an image generated as a function of a reflection of a sound wave off of a structure. Non-limiting examples of ultrasonic images and/or imaging techniques include intracardiac echo (ICE) images, transthoracic echocardiograms (TTE), transesophageal echocardiograms (TEE), and point of care ultrasound (POCUS). As used herein, a “structure” is a component of a subject. Non-limiting examples of structures include organs and tissues. In non-limiting examples, a structure may include a heart, lung, spleen, liver, kidney, muscle, skeleton, intestine, stomach, vein, and/or artery. In additional non-limiting examples, a structure may include a left atrium, left atrial appendage, left ventricle, right ventricle, and/or a right atrium. A structure may be a solid organ, a hollow organ, a vascular component, or a musculoskeletal element that contributes to the overall function of a biological system. In non-limiting examples, a structure may include the heart, which consists of multiple chambers and associated vessels responsible for circulating blood throughout the body. In some embodiments, a structure may include components of the respiratory system, such as the lungs, which facilitate gas exchange through an intricate network of alveoli and capillaries. In some embodiments, a structure may include vascular components, such as arteries, which carry oxygenated blood from the heart to various tissues, and veins, which return deoxygenated blood to the heart. In additional non-limiting examples, a structure may include elements of the digestive system, such as the stomach, which aids in digestion through mechanical and chemical processes, and the intestines, which are divided into the small intestine (duodenum, jejunum, ileum) and large intestine (cecum, colon, rectum). In some embodiments, a structure may include neurological components, such as the brain, spinal cord, and peripheral nerves. In further non-limiting examples, a structure may include components of the musculoskeletal system, such as bones, muscles, tendons, and ligaments.

In an embodiment, set of images may include a set of intracardiac echocardiography (ICE) images. As used herein, a “set of ICE images” is a collection of ultrasound images obtained from within the heart's chambers or blood vessels. In some cases, ICE images may be captured using a specialized catheter equipped with an ultrasound transducer that is inserted into the body and guided to the heart of subject. In an embodiment, set of images may provide a detailed and real-time visualizations of cardiac anatomy. As used herein, “cardiac anatomy” is the structural composition of the heart and its associated blood vessels. Set of images may also include internal structures, functions, and blood flow patterns of the heart of subject. Other exemplary embodiments of set of images may include, without limitation, X-ray images, magnetic resonance imaging (MRI) scans, computed tomography (CT) scans, ultrasound images, optical images, digital photographs, or any other form of visual data. Additionally, images within set of images may be related in terms of content, time of capture, sequence, or any other relevant parameters described herein. In a non-limiting example, each image of set of images may represent a particular view, angle, or perspective of an object, subject, or scene, and may be in two-dimensional (2D) or 3D format. Images of set of images may include, without limitation, any two-dimensional or three-dimensional images of any anatomy or anatomical structure, including without limitation images of any internal organ, tissue including without limitation muscular, connective tissue, epithelial tissue, and/or nervous tissue, bone, and/or any other element that may be imaged within a human and/or animal body.

Still referring to FIG. 1, in a non-limiting example, structure may include chambers (e.g., four chambers including left and right atria and left and right ventricles), valves (i.e., the structures that regulate blood flow between chambers and vessels, including mitral, tricuspid, aortic, and pulmonary valves), vessels (e.g., aorta, pulmonary arteries and veins, and coronary arteries), conduction system (i.e., a network of specialized cells that control the heart's electrical activity and rhythm), muscular and connective tissues (e.g., heart's muscular walls, septa, any other connective tissues that provide structural integrity and enable contraction), LAA and other appendages, pathological features (e.g., any abnormalities, defects, and/or the like), and/or other components of a heart.

Still referring to FIG. 1, as used in this disclosure, a “subject” refers to an individual organism. In an embodiment, subject may include a human, such as a human undergoing a medical procedure such as atrial fibrillation (AF) ablation. In some cases, subject may include a provider of set of images described herein. In other cases, subject may include a recipient or a participant in a clinical trial or research study. In a non-limiting example, subject may include a human patient with AF who is undergoing a procedure, an individual undergoing cardiac screening, a participant in a clinical trial, patient with congenital heart disease, heart transplant candidate, patient receiving follow-up care after cardiac surgery, healthy volunteer, patient with heart failure, or the like. Additionally, or alternatively, subject may include an animal model (i.e., animal used to model AF such as a laboratory rat).

Still referring to FIG. 1, in an embodiment, each ultrasonic image of set of ultrasonic images may include a particular view of subject's heart's chambers, valves, vessel, and/or the like. In a non-limiting example, set of images may include multiple views e.g., different angles and perspectives of subject's heart. In another embodiment, set of images may be arranged in a temporal sequence. In a non-limiting example, set of images may include a series of images captured over time, allowing for an observation of dynamic cardiac functions such as beating, blood flow, and/or the like. In some cases, each ultrasonic image of set of images may include a corresponding timestamp, wherein the timestamp may include an indicator showing a date and time of when the corresponding ultrasonic image was taken.

With continued reference to FIG. 1, an agent may be configured to generate a set of shape parameters based on set of images. As used in this disclosure, a “set of shape parameters” refers to a collection of numerical values or descriptors that quantitatively represent the geometric or morphological characteristics of a structure. In some embodiments, a set of shape parameters may represent a shape of a structure. In a non-limiting example, set of shape parameters may include information and/or metadata calculated, determined, and/or extracted from set of ultrasonic images, such as, dimensions, angles, curvatures, surface areas, texture, symmetry, and/or the like. In other embodiments, agent may be configured to parameterize features (e.g., edges, textures, contours, and any other characteristics that describe the shape structure) extracted from set of images using CNN described herein. Such parameterization may involve agent deriving one or more shape parameters including one or more morphological descriptors that quantitatively describe structure based on extracted features. In some cases, agent may be configured to use principal component analysis (PCA) to reduce the dimensionality of set of shape parameters, allowing agent to focus on the most informative shape parameters of set of shape parameters in further processing steps described below.

With continued reference to FIG. 1, in a non-limiting example, set of shape parameters may be generated based on set of images using machine learning model such as, without limitation, a shape identification model. Generating set of shape parameters may include receiving structure training data, wherein the structure training data may include a plurality of image sets as inputs correlated to a plurality of shape parameter sets as outputs. In some cases, structure training data may be received from a data store. For example, and without limitation, structure training data may be used to show each ultrasonic image may indicate a particular set of shape parameters. In some embodiments, structure training data may include historical ultrasonic images correlated with historical computed tomography scan data. Such a training dataset may be used to train shape identification model to generate a set of shape parameters representing a structure's shape as a function of a set of ultrasonic images, which may be input into the model in order to receive, as an output, a set of shape parameters. Shape identification model may be trained by an agent using structure training data. Additionally, structure training data may include previously input image sets and their corresponding shape parameter outputs. Shape identification model may be iterative such that outputs may be used as future inputs of shape identification model. This may allow the shape identification model to evolve. An agent may be further configured to generate set of shape parameters as a function of set of images using the trained shape identification model.

Still referring to FIG. 1, generating set of shape parameters may include performing image processing/segmentation techniques, as described above, prior to implementation of shape identification model in order to optimize performance and runtime of an agent and training of a model. For example, image segmentation may include normalization and standardization methods performed by computer vision model to ensure that pixel values in images are normalized or standardized to a consistent scale thus aiding convergence during training of shape identification model. Image segmentation may include data augmentation techniques such as rotation, scaling, flipping, and translation to artificially increase the size of the training dataset and improve model generalization. Image segmentation may include image enhancement preprocessing techniques like histogram equalization or contrast stretching to enhance relevant features in the images. Image segmentation may include texture and shape descriptors to extract features beyond pixel values, such as texture and shape descriptors, to capture additional information about structures. Image segmentation may include architecture selection methods, as in experiments with different architectures, such as U-Net, DeepLab, or custom architectures, depending on the complexity and characteristics of the images. Image segmentation may include grid Search or random Search processing methods to systematically explore hyperparameter combinations to find the optimal configuration for a 3D model. As previously disclosed, image segmentation may include separating specific structures or regions of interest (ROI) from the background or other structures in a given ultrasonic image, wherein a collection of ROIs may be also incorporated by the shape parameter training data/structure training data.

With continued reference to FIG. 1, an agent may use a statistical shape model to generate and/or iteratively refine a 3D model based on a set of shape parameters. As used herein, a “3D model,” is a 3D representation of a structure. In some embodiments, a 3D model may include a heart model. A heart model may include a 3D representation of cardiac anatomy. In some cases, 3D model may be generated through a direct 3D reconstruction from a series of (2D) ultrasonic images. In a non-limiting example, set of images may include a plurality of ultrasonic images captured from different angles and positions within and/or around a structure. An agent may be configured to apply one or more 3D reconstruction algorithms, such as without limitation, marching cubes, contour detection and segmentation, active contour models, and/or the like to create a coherent 3D representation e.g., 3D model of structure. In some cases, such direct 3D reconstruction may leverage the inherent spatial information within set of images, providing a direct and intuitive way to model the 3D model of a structure. In a further embodiment, generic 3D modeling techniques may be applied to create the initial 3D model. In some cases, generic 3D modeling techniques may include surface modeling, solid modeling, or parametric modeling, among others. As an ordinary person skilled in the art, upon reviewing the entirety of this disclosure, will be aware of various 3D reconstruction algorithms that may be used by an agent to generate 3D model of structure. As used in this disclosure, a “statistical shape model” (SSM) is a data structure representing, including, and/or utilizing a mathematical model that captures principal modes of variation in shape across a population of structures. In some cases, SSM may be constructed by analyzing one or more datasets of shapes and identifying, for example, mean shapes and main modes of variation within the one or more datasets. In a non-limiting example, SSM may start with calculation of at least one mean shape, which represents an average geometry of all shapes of a structure in a given dataset, wherein the at least one mean shape may be served as a central reference point for an agent to understand different variations. In some embodiments, unique SSMs are created for different structure categories, such as different organs or tissues. In a non-limiting example, a first SSM may be created for a first structure category such as kidneys and a second SSM may be created for a second structure category such as hearts. In some cases, dataset may include, without limitation, structure training data, structure training data, and/or any datasets within ultrasonic image databases described herein. SSM may also identify one or more principal modes of variation within given datasets described herein, wherein the “principal modes of variations,” for the purpose of this disclosure, refer to main patterns or directions along which data points vary within dataset. In a non-limiting example, identifying principal modes of variations may include applying principal component analysis (PCA) on given dataset. Additionally, or alternatively, shapes may be described directly using plurality of shape parameter sets (in structure training data). In some cases, shape parameter sets may correspond to a plurality of modes of variations. Further, one or more statistical constraints (e.g., mean, variance, correlation, boundary, proportion constraint and/or the like) may be introduced into SSM based on the distribution of shape parameters within plurality of shape parameter sets and/or 3D structure dimensions. In some embodiments, each shape parameter within a set of shape parameters may be associated with and/or comprise a corresponding parameter range. Such a parameter range may, for example, include a range of values associated with a normal and/or healthy structure. Such a parameter range may be determined based on, for example, a subset of possible values of a parameter which historical healthy structures commonly fall into, as determined from a dataset.

With continued reference to FIG. 1, in some cases, once modes of variation are extracted, an agent may be configured to create a shape representation for any given structure shape within the studied class. In a non-limiting example, 3D model having a shape S may be mathematically represented as

$$S = \overline{S} + {Σ}_{k = 1}^{M} {a}_{k} \times {ϕ}_{k} ,$$

wherein S̅ denotes the mean shape derived from the set of example shapes, M is the number of modes of variation considered, aₖ are the coefficients or weights for each mode, and ϕₖ are the modes of variation (eigenvectors corresponding to the kth principal component). In some cases, coefficients aₖ may dictate a degree to which each mode of variation is present in shape S. In some cases, coefficients aₖ may vary from positive to negative (or negative to positive) based on the deformation of the 3D model in directions described by each mode of variation. In some cases, 3D model may include mean shape as described herein. In some cases, 3D model may include a predictive structure shape that may not have been explicitly seen in the set of example shapes or patient's heart observations. In some cases, 3D model may be in 3D VOR as described above.

Still Referring to FIG. 1, an agent may be configured to generate a map regarding one or more levels of uncertainty. A “map,” as used herein, refers to a visualization. Map may include level(s) of uncertainty to be visualized on the 3D model. Map may include a color-coded heatmap, including other visual cues, symbols or indicators that alert a user to areas of 3D model that may require extra caution when used for planning or guidance during a medical procedure. For example, after obtaining the segmentation results from 3D model, map may be generated. Map may highlight the uncertainty or confidence level associated with each pixel in the segmentation. Assigning colors to different intensity levels in map allows for an intuitive visualization. Typically, warmer colors (e.g., red, or yellow) might represent high uncertainty, while cooler colors (e.g., blue, or green) could indicate low uncertainty. The color-coding can be adjusted based on specific thresholds or clinical requirements.

Still referring to FIG. 1, generating map may include methods such as Class Activation Mapping (CAM). Class Activation Mapping is a technique that originated for image classification tasks and has been extended to provide visual insights into the regions of an image that are most important for a particular class. CAM allows the visualization of the spatial attention of a convolutional neural network (CNN) by generating heat maps that highlight discriminative regions. CAM may be applied to the last convolutional layer of a CNN. The features extracted by this layer capture high-level semantic information, making it suitable for visualizing the importance of different regions in an image. CAM is typically applied to the last convolutional layer of a CNN. The features extracted by this layer capture high-level semantic information, making it suitable for visualizing the importance of different regions in an image. The output of the global average pooling is then fed into a fully connected layer with a softmax activation function. This converts the features into class scores, indicating the likelihood of the image belonging to different classes. The CAM algorithm computes a weighted sum of the original feature maps based on the weights of the fully connected layer. These weights are determined during the training process and represent the importance of each feature map for a specific class. The weighted sum is applied to the original feature maps, producing a single heat map. This heat map highlights the regions of the input image that contributed most to the prediction for the target class. The generated heat map can be overlaid on the input image, visually indicating which regions are most relevant for the predicted class. Typically, warmer colors (e.g., red, or yellow) represent higher activation or importance.

Still Referring to FIG. 1, generating map may include Grad-CAM (Gradient-weighted Class Activation Mapping). Grad-CAM is an extension of Class Activation Mapping (CAM) that enhances the localization capabilities by incorporating gradient information from the final convolutional layer of a neural network. Grad-CAM helps to generate heat maps that highlight discriminative regions in an image, providing more fine-grained insights into where α convolutional neural network (CNN) is focusing its attention when making predictions. In traditional CAM, the last convolutional layer's feature maps are linearly combined to obtain a weighted sum, and the resulting weights are used to create a heat map that highlights relevant regions for a specific class. Grad-CAM improves upon CAM by introducing gradient information. It computes the gradients of the predicted class score with respect to the feature maps of the last convolutional layer. Grad-CAM retains the global average pooling (GAP) operation applied after the last convolutional layer, as it is an integral part of CAM. The GAP operation condenses the spatial information into a single value per feature map. The gradients obtained in the previous step are used to calculate the importance of each feature map. These gradients represent the importance of each feature map in contributing to the final prediction. A weighted sum is computed using these gradients, and this is combined with the original feature maps. The computed sum goes through a ReLU activation function, discarding any negative values. This step emphasizes positive contributions and suppresses negative ones. The ReLU-activated weighted sum is linearly combined with the original feature maps to produce a weighted combination. This combination retains spatial information and helps create a more accurate heat map. The resulting heat map is often normalized to enhance visualization, ensuring that the values are within a specific range (e.g., between 0 and 1). The final heat map generated by Grad-CAM is then overlaid on the input image, highlighting the regions of interest for the predicted class. The intensity of the heat map indicates the importance of different regions. Grad-CAM enhances the interpretability and explainability of deep learning models, allowing practitioners and researchers to understand which parts of an image are crucial for a particular prediction. This is particularly valuable in applications such as medical imaging or any domain where understanding the decision-making process is critical.

Still Referring to FIG. 1, generating map may include utilizing a “SmoothGrad technique,” a technique designed to improve the interpretability of neural network predictions by reducing the noise in the attribution maps or heat maps generated by visualizing gradients. It is particularly useful for understanding the decision-making process of deep learning models, especially in scenarios where the explanations need to be robust and less sensitive to input perturbations. The primary goal of SmoothGrad is to enhance the visual quality of attribution maps generated by visualizing gradients. Attribution maps highlight the regions in the input that contribute most to a model's prediction. SmoothGrad aims to reduce the impact of noise in these maps, providing more stable and interpretable visualizations. The key idea behind SmoothGrad is to introduce perturbations to the input data. Instead of attributing the prediction solely to the gradients calculated with respect to the original input, the gradients are averaged over multiple perturbed versions of the input. By averaging the gradients over multiple perturbed samples, SmoothGrad helps reduce the impact of noise or irrelevant features in the attribution maps. This is particularly beneficial when dealing with complex or noisy datasets. Perturbation techniques include adding Gaussian noise, random rotations, or random translations to the input data. These perturbations create variations in the input while preserving the essential features, leading to more stable and reliable attribution maps. For each perturbed input, gradients are calculated with respect to the model's output. These gradients are then averaged over all perturbed samples. This process smoothens the attribution map by reducing the influence of random noise. The averaged gradients may undergo normalization or scaling to ensure that the values are interpretable and within a specific range. This step can enhance the consistency and comparability of the generated attribution maps. The final step involves generating a heat map using the smoothed gradients. The heat map represents the attribution of different regions in the input to the model's prediction, providing a clearer and more stable visualization.

Still Referring to FIG. 1, generating map may include implementing one or more Gaussian Processes. A Gaussian Process is a collection of random variables, any finite subset of which has a joint Gaussian distribution. In simpler terms, it's a distribution over functions rather than a distribution over finite-dimensional vectors. Gaussian Processes (GPs) can be applied to generate heat maps in various ways, particularly in the context of regression tasks where one would want to predict continuous values across a spatial domain. Given a set of observed data points, the GP can predict the values at unobserved locations in the spatial domain. Importantly, it also provides uncertainty estimates associated with these predictions. This uncertainty can be visualized as a heat map. The predicted values from the GP represent the main heat map, indicating the expected values across the spatial domain. The uncertainty associated with each prediction can be visualized as an uncertainty heat map. This uncertainty heat map provides insights into regions where the model is less confident about its predictions. Overlay of the main heat map and the uncertainty heat map on the original spatial data may create a composite visualization. Warmer colors in the main heat map might represent higher predicted values, while the uncertainty heat map's intensity could indicate regions where the model's predictions are less certain.

Still referring to FIG. 1, in some embodiments, an agent may be used to generate a Pulsed Field Ablation (PFA) durability datum. In some embodiments, an agent may include a lesion durability agent. As used herein, a “lesion durability agent” is an agent configured to generate a PFA durability datum. In some embodiments, a lesion durability agent may generate a PFA durability datum as a function of a PFA device parameter using a trained PFA durability machine learning model. In some embodiments, an agent output may include such PFA durability datum. In some embodiments, procedure data may include a Pulsed Field Ablation (PFA) device parameter and an agent may determine the agent output 168 by generating a PFA durability datum as a function of the PFA device parameter using a trained PFA durability machine learning model. In some embodiments, such a process may be consistent with U.S. patent application Ser. No. 18/646,991 (having attorney docket number 1518-142USU1), filed on Apr. 26, 2024, and titled “METHOD AND APPARATUS FOR PREDICTING PULSED FIELD ABLATION DURABILITY,” the entirety of which is hereby incorporated by reference.

Still referring to FIG. 1, in some embodiments, an agent may receive PFA data. As used herein, “PFA data” is medical data of a subject which undergoes PFA, including PFA device parameters. PFA includes the delivery of rapid high voltage pulsed electrical fields to tissue, such as cardiac tissue. This may cause electroporation of cell membranes in the affected tissue. In some embodiments, PFA may include irreversible electroporation, in which pores are created in cell membranes, leading to cell death. In some embodiments, the strength of the effect applied may be controlled such that only target tissues are destroyed, and not surrounding tissues. In some embodiments, surrounding tissues around a target tissue may have higher thresholds for damage from electroporation. PFA may be applied to subject, and PFA data of subject may be determined. In some embodiments, PFA may be applied in subject with Atrial Fibrillation (AFib).

Still referring to FIG. 1, in some embodiments, PFA data may include PFA device parameter. As used herein, a “PFA device parameter” is an input variable that can be used to control output of a PFA device. As used herein, a “PFA device” is a device used to perform pulse field ablation of tissue, e.g., cardiac tissue. Non-limiting examples of PFA device include the FARAPULSE PFA System (Boston Scientific) and PulseSelect (Medtronic). Non-limiting examples of PFA device parameters include voltage, pulse duration, frequency, pulse width, amplitude, power of ablation, total energy delivered, total treatment time, energy delivered to a particular location, treatment time at a particular location, current, average power, peak power, and pulse delivery phase (e.g., biphasic vs monophasic pulse delivery). In some embodiments, a PFA device parameter may be selected from the list consisting of voltage, pulse duration, frequency, pulse width, amplitude, power of ablation, total energy delivered, total treatment time, energy delivered to a particular location, treatment time at a particular location, current, average power, peak power, and biphasic vs monophasic pulse delivery. In some embodiments, PFA data may include a PFA device identifier. As used herein, a “PFA device identifier” is a representation of a type of PFA device used to perform PFA in a subject. In some embodiments, PFA data may include an electrode configuration used to apply PFA. In some embodiments, PFA data may include a location of one or more electrodes during PFA. In some embodiments, PFA device parameter may include a parameter that has been used, is about to be used, and/or could be used at a PFA device. In some embodiments, an agent may receive from PFA device PFA device parameter. In some embodiments, an agent may input into PFA device a PFA device parameter. For example, a PFA device parameter may be generated, optimized and/or modified based on a function described herein, and a result may be transmitted to PFA device for use in a PFA procedure.

Still referring to FIG. 1, in some embodiments, PFA data may include electrocardiogram (ECG) datum. As used herein, an “ECG datum” is a datum describing electrical activity of a heart. Likewise, “ECG data” is data describing electrical activity of a heart. In some embodiments, an ECG datum may include a rhythm strip ECG datum. As used herein, a “rhythm strip ECG datum” is a datum describing electrical activity detected using a single electrode. In some embodiments, an ECG datum may include a median beat ECG datum. As used herein, a “median beat ECG datum” is a datum describing electrical activity detected using a plurality of leads and/or electrodes. In some embodiments, ECG datum may include data collected by 2, 3, 4, 5, 6, 7, 8, 9, 10, 11, 12, or more ECG leads. For example, ECG datum may include a median beat collected by 12 ECG leads. In some embodiments, ECG datum may be associated with subject. In some embodiments, ECG datum may be detected and/or recorded using ECG sensor. ECG sensor may include one or more electrodes. Electrodes may be placed on subject such as on chest, arms, and legs of subject. Electrodes may detect electrical impulses produced by the heart. Lead wires may be used to connect electrodes to a computing device of an ECG sensor. ECG sensor may receive electrical signals from electrodes, may amplify such signals and convert them into a visual representation, such as a waveform. ECG sensor may include one or more lead wires. As used herein, an “ECG sensor” is a device configured to measure the electrical activity of a heart. ECG sensor may include a device configured to measure and/or interpret electrical activity of heart of subject using electrodes and/or lead wires. In some embodiments, ECG sensor may be configured to detect ECG datum and/or transmit ECG datum to an agent. In some embodiments, ECG sensor may include a surface ECG sensor. In some embodiments, ECG sensor may include an intracardiac ECG sensor.

Still referring to FIG. 1, in some embodiments, PFA data may include image data. Such image data may include cardiac image data. Cardiac image data may be obtained by, in non-limiting examples, echocardiogram, cardiac computed tomography, nuclear cardiac stress test, single-photon emission computed tomography, cardiac positron emission tomography, coronary angiogram, cardiac MRI, and multigated acquisition scan.

Still referring to FIG. 1, in some embodiments, an agent may generate PFA durability datum as a function of PFA data using a trained PFA durability machine learning model. As used in this disclosure, a “PFA durability machine learning model” is any machine-learning model, process, or algorithm that outputs a PFA durability datum. As used herein, a “PFA durability datum” is a representation of PFA durability, for example measurement, quantification, prediction, estimate, and/or probability of PFA durability. As used herein, “PFA durability” is a tendency for tissue treated with pulsed field ablation to remain affected by the pulse field ablation, e.g., stay dead. PFA durability machine learning model may be trained using a supervised learning algorithm. PFA durability machine learning model may be trained on training data including example PFA data, associated with example PFA outcomes. As used in this disclosure, a “PFA outcome” is used to describe both (1) physiological results of PFA procedures; and (2) representation of these results. PFA outcomes may be represented according to medical device measurements (e.g., ECG), doctor notes, patient accounts, and the like. In some cases, example PFA data may include example PFA device parameters. Once PFA durability machine learning model is trained, it may be used to determine PFA durability datum. An agent may input PFA data into PFA durability machine learning model, and an agent may receive PFA durability datum from the model. In a non-limiting example, PFA durability machine learning model may be trained on a training dataset including example PFA device parameters associated with example PFA outcomes, and PFA durability machine learning model may accept as an input PFA device parameter. In another non-limiting example, PFA durability machine learning model may be trained on a training dataset including example ECG data associated with example PFA outcomes, and PFA durability machine learning model may accept as an input ECG datum. In some embodiments, training dataset for PFA durability machine-learning model may include ECG data and/or electrogram (EGM) data correlated to PFA outcomes. In some embodiments, training dataset for PFA may include in-procedure ECG data and/or in-procedure electrocardiogram data correlated to PFA outcomes. For the purposes of this disclosure, “in-procedure ECG data” refers to data that is collected using an ECG during an ablation procedure. For the purposes of this disclosure, “in-procedure EGM data” refers to data that is collected using an EGM during an ablation procedure. In some embodiments, PFA durability machine learning model may include a fused classifier ensemble machine learning model. For example, the output of one or more classifiers may be used as inputs in another machine learning step. Such a system may use gradient boosting, such as, in a non-limiting example, CatBoost. In some embodiments, PFA durability machine learning model may include a 1-dimensional convolutional neural network, such as, in a non-limiting example, U-Net. In some embodiments a 1-dimensional convolutional neural network may be used to interpret intracardiac voltage and/or surface voltage input data. In some embodiments, PFA durability machine learning model may include a 2-dimensional convolutional neural network. In a non-limiting example, a 2-dimensional convolutional neural network may be used to interpret CT scans and/or MRI scans to create classifications between geometric surfaces which may be predictive of Afib recurrence.

Still referring to FIG. 1, in some embodiments, PFA durability datum may include a likelihood and/or probability that a lesion is predicted to be durable. In some embodiments, a probability that a lesion is predicted to be durable is expressed as a number from 0 to 1. In some embodiments, a probability that a lesion is predicted to be durable is expressed as a percentage. In some embodiments, PFA durability datum may include a length of time over which a lesion is predicted to be durable. In some embodiments, PFA durability datum may include a Boolean variable representing whether or not a lesion is predicted to be durable. In some embodiments, PFA durability datum represented on a continuum may be mapped to one or more fuzzy sets representing values of linguistic variables.

Still referring to FIG. 1, in some embodiments, example PFA outcomes may be determined from patient medical records. Example PFA outcomes may include diagnoses, such as whether AFib recurred and/or resolved, and ECG data, such as ECG data of a form described above. In some embodiments, example PFA outcomes may be determined from image data, such as image data generated using, in non-limiting examples, echocardiogram, cardiac computed tomography, nuclear cardiac stress test, single-photon emission computed tomography, cardiac positron emission tomography, coronary angiogram, cardiac MRI, and multigated acquisition scan. In some embodiments, example PFA outcomes may be determined using intracardiac echocardiography (ICE). In some cases, example PFA outcomes may include a date of recurrence of AFib and/or a date of reperformed ablation. In some embodiments, such dates may be measured in absolute terms and/or in terms relative to a date of a PFA procedure. In some embodiments, PFA outcome data may include an AFib burden. As used herein, an “AFib burden data” is a representation of AFib experienced by a subject, for example quantity of Afib, quality of Afib, or both. In some embodiments, low AFib burden may correlate with positive example PFA outcomes as, in some cases, minor occasional AFib may not warrant a second cardiac ablation, for example. In some embodiments, example PFA data may include historical PFA data in a form described above and/or gathered as described above with respect to PFA data. In some embodiments, example PFA outcomes may be determined based on medical data of subjects captured after such subjects undergo PFA. In a non-limiting example, example PFA outcomes may include historical post-PFA procedure ECG data. As used in this disclosure, “post-PFA procedure ECG data” is a representation of an any electrocardiogram-type of measurement taken on a subject, at any time, after a pulse field ablation procedure. Additional detail with respect to timing of data gathering is provided below. In some embodiments, example PFA outcomes may be categorical, such as positive or negative outcome. In some embodiments, example PFA outcomes may have a numerical value, such as a value within a range where values on one end of the range indicate positive outcomes and values on the other end of the range indicate negative outcomes.

Still referring to FIG. 1, in some embodiments, an agent may be used to process an electrocardiogram (ECG) datum, such as by determining a signal metric based on the ECG datum. In some embodiments, system 100 may receive, from user interface 124, user input 128, receive an ECG datum, determine, using agent orchestrator 156, an agent selection datum by generating the agent selection datum as a function of user input 128 using a trained agent selection machine learning model, using an agent corresponding to the agent selection datum, determine agent output 168 by inputting into the third agent the ECG datum, and receiving, as an output from the agent, a signal metric, and display, using user interface 124, the signal metric.

Still referring to FIG. 1, in the context of ECG data, a signal may include a physical record of medical data of a subject. In some embodiments, signal may include a paper readout of medical data produced by a device which records such data from a sensor. Signal may include, in non-limiting examples, electrocardiogram (ECG) data, electroencephalogram (EEG) data, X-ray data, MRI data, CT scan data, and pathology test data. In a non-limiting example, signal may include a physical printout of such data. In some embodiments, signal may include a measurement of activity of a subject's heart. In some embodiments, signal may include ECG data. In some embodiments, signal may include time series data. In some embodiments, signal may include a plurality of parallel recordings of time-series data, such as in a 12 lead ECG.

Still referring to FIG. 1, in some embodiments, an agent may determine signal metric as a function of an image. As used herein, a “signal metric” is a measurement of a signal, a measurement of a feature of a signal, or both. In non-limiting examples, where α signal includes ECG data, a signal metric may include a measurement of a PR interval, RR interval, ST interval, TP interval, QT interval, P wave duration, PR segment, QRS duration, ST segment, P axis, and number of beats per minute. In another example, where α signal includes ECG data, a signal metric may include a rhythm type, such as a sinus rhythm. In some embodiments, signal metric is selected from the list consisting of a PR interval, a QRS duration, a P axis, and a number of beats per minute. In a non-limiting example, signal metric may include a measurement of a first feature of a signal relative to a second feature of a signal. Signal metric may be determined using a machine vision system. For example, a machine vision system may be used to determine one or more peaks of ECG data, and a distance between peaks may be used to determine an RR interval. In another example, a machine vision system may be used to determine a slope of one or more points and/or segments of ECG data and/or rate of change of such a slope, and such data may be used to determine a QRS duration. In some embodiments, signal metric may be determined using a signal metric machine learning model. In some embodiments, a signal metric machine learning model may be trained using a supervised learning algorithm. A signal metric machine learning model may be trained on a training dataset including example images, associated with example signal metrics. Such a training dataset may be generated by, for example, collecting images of signals, and associating them with historical signal metrics manually determined by specialists based on those signals. In some embodiments, generation of signal metric may include embedding image. Embedding image may include generation of a numerical representation of image. In some embodiments, such a numerical representation may include a vector, where similarity between vectors across multiple inputs indicate similarity between inputs. In some embodiments, a machine learning model, such as a convolutional neural network, may be used to create such a numerical representation. Non-limiting examples of convolutional neural networks for embedding image data include VGG (Visual Geometry Group), ResNet (Residual Networks), Inception (GoogLeNet) and EfficientNet. In some embodiments, one or more preprocessing steps may be applied prior to embedding image. For example, image may be resized and/or normalized in order to make it suitable for input into a machine learning model trained to generate an embedding. In some embodiments, embedding image data may be used to reduce dimensionality of high dimensional data. In some embodiments, embedding image data may be used to extract features from image data. In some embodiments, an embedding may be input into signal metric machine learning model, and signal metric may be received as an output.

Still referring to FIG. 1, in some embodiments, an agent may determine signal metric position as a function of signal metric. As used herein, a “signal metric position” is a data structure describing the position of a signal metric relative to that of one or more members of a population. As a non-limiting example, a signal metric position may indicate that a subject's PR interval is higher than 55% of a population. In some embodiments, a population restriction may be identified, and a population which a user's signal metric is compared to may be determined according to a population restriction. As used herein, a “population restriction” is a data structure setting a boundary on individuals to be considered members of a population. In non-limiting examples, population restrictions may include a limitation that members of a population be male, and a limitation that members of a population be under 25 years old. In a non-limiting example, determination of signal metric position may include the following steps: determination of signal metric as described herein, retrieval of a plurality of instances of a like metric of members of a population conforming to a population restriction or retrieval of data describing a distribution of such metric among members of a population, and comparison of signal metric to such metrics. In some embodiments, retrieval of a like metric of members of a population and/or retrieval of data describing a distribution of such metric may include generation of a query requesting such information from a database, such as repository, transmission of such query to repository, and receipt of a response. In a non-limiting example, signal metric may be compared to like metrics of members of a population in order to determine a percentage of like metrics which signal metric is above.

Still referring to FIG. 1, in some embodiments, an agent may generate abnormality datum. In some embodiments, abnormality datum may be generated as a function of image. As used herein, an “abnormality datum” is a data structure describing a difference between a signal and a typical signal of a healthy individual. In a non-limiting example, abnormality datum may include an amount a subject's at rest heart rate is above an at rest heart rate of a healthy individual. In some embodiments, abnormality datum may be determined as a function of signal metric and/or signal metric position. In some embodiments, an agent may generate abnormality datum based on signal metric being above or below a threshold. A threshold may be determined as a function of information about a subject associated with signal, such as age, sex, medical history, and the like. In another non-limiting example, an agent may generate abnormality datum based on signal metric position being above or below a threshold. In a non-limiting example, an agent may generate abnormality datum if signal metric position indicates that signal metric is in the top 5% of a population. In some embodiments, system 100 may display an abnormality datum using user interface 124.

Still referring to FIG. 1, in some embodiments, an agent may determine medical condition datum. As used herein, a “medical condition datum” is a data structure identifying in a subject an ailment, a lack of an ailment, a likelihood of an ailment, or a combination thereof. For example, medical condition datum may indicate that a subject has a particular disease. In another example, medical condition datum may indicate that a subject does not have a particular disease. In another example, medical condition datum may indicate that a subject is healthy. In another example, medical condition datum may indicate that a subject has a first disease and does not have a second disease. In another example, medical condition datum may indicate that a subject has a high likelihood of having a particular disease. Diseases which medical condition datum may identify include, in non-limiting examples, an infectious disease, a deficiency disease, a hereditary disease, and a physiological disease. In some embodiments, an agent may display medical condition datum to user. Display of information to a user is described below.

Still referring to FIG. 1, in some embodiments, an agent may determine medical condition datum by identifying a similarity between signal metric and deidentified patient health information of repository and generating medical condition datum as a function of the similarity. As used herein, a “similarity” between a first datum and a second datum is a data structure describing the numerical distance between the first datum and the second datum, a data structure describing whether the first datum and the second datum are members of the same category, or both. As a non-limiting example, a similarity may include a comparison between a first subject's heart rate while resting with heart rates while resting of a population. In some embodiments, a similarity may be determined between abnormality datum and deidentified patient health information of repository, and medical condition datum may be generated as a function of such similarity. In some embodiments, a similarity may be determined which accounts for multiple signal metrics and/or other information relating to a subject such as age, sex, ethnicity, levels of physical activity, diet, medications the subject is on, and other aspects of subject's medical history. In a non-limiting example, an agent may determine signal metric from signal, query repository for deidentified patient health information with metrics within a range of signal metric, receive deidentified patient health information from repository, and determine medical condition datum as a function of medical conditions of received deidentified patient health information.

Still referring to FIG. 1, in some embodiments, an agent may generate medical condition datum using a medical condition machine learning model. Medical condition machine learning model may be trained using a supervised learning algorithm. Medical condition machine learning model may be trained on a training dataset including example images, signal metrics, abnormality data, and/or calibration data, associated with example medical conditions. Such a training dataset may be obtained by, for example, gathering diagnoses of historical subjects and associating those diagnoses with images of ECG data of those subjects. Once medical condition machine learning model is trained, it may be used to determine medical condition datum. An agent may input image, signal metric, calibration datum, and/or abnormality datum into medical condition machine learning model, and an agent may receive medical condition datum from the model.

Still referring to FIG. 1, in some embodiments, an agent may generate medical condition confidence score. In some embodiments, medical condition machine learning model may output medical condition confidence score in addition to its other outputs. As used herein, a “confidence score” is a degree of confidence that an associated datum is accurate. As used herein, a “medical condition confidence score” is a degree of confidence that a medical condition datum is accurate. In some embodiments, a confidence score may be determined as a function of a machine learning model, such as medical condition machine learning model. Confidence scores may be used to predict how likely a model output is to be accurate. For example, in some classifiers, numerical values are calculated, and a cutoff value is used to determine which category the input fits into. In this example, the numerical value may be used to determine a certainty score based on how closely it fits into a class and/or how close to a decision boundary it is. In another example, in clustering algorithms, certainty scores may be calculated based on how closely an input fits into a cluster. In some embodiments, medical condition datum may be generated without the use of medical condition machine learning model, and medical condition confidence score may be generated using other methods. For example, where medical condition datum is determined as a function of a comparison between signal metric and a threshold, medical condition confidence score may be determined as a function of the distance between signal metric and the threshold. In a non-limiting example, signal may include ECG data, signal metric may include a prediction of a subject's left ventricular ejection fraction (LVEF) based on such ECG data, and abnormality datum may be determined based on a comparison between the LVEF prediction and a threshold. For example, abnormality datum may be determined if such LVEF prediction is below a threshold.

Still referring to FIG. 1, in some embodiments, an agent may select medical condition machine learning model from a plurality of medical condition machine learning models. In some embodiments, such selection may be performed as a function of calibration datum. In a non-limiting example, different medical condition machine learning models may be applied to images of different signal types, and calibration datum may indicate a type of signal that image depicts (such as ECG data), such as based on user input.

Still referring to FIG. 1, in some embodiments, an agent may identify guidance on treatment of a medical condition as a function of medical condition datum. For example, an agent may retrieve from a database guidance on best practices for treatment and/or prevention of a medical condition associated with medical condition datum. In some embodiments, retrieved guidance may include guidance published by a relevant medical association. In some embodiments, an agent may identify guidance using a web search, such as a keyword search. In some embodiments, an agent may identify guidance using a machine learning model, such as a language model trained on medical publications. Guidance on treatment of a medical condition may be displayed to user.

Still referring to FIG. 1, in some embodiments, an agent may generate quality diagnostic of image. In some embodiments, quality diagnostic is generated by extracting a plurality of signal metrics from signal; validating signal by classifying signal to a plurality of preliminary signal metrics; and determining an accuracy status of the extracted plurality of signal metrics by comparing the plurality of preliminary signal metrics to the extracted plurality of signal metrics; and generating the quality diagnostic based on validation of signal. In some embodiments, quality diagnostic may identify an error in a medical procedure used to record signal, and/or an error in another step such as capturing of image of signal, and/or processing of image. In some embodiments, an agent may alert user as to an error identified by quality diagnostic. This may allow user to, for example, record a new, more accurate, set of data. For example, an agent may capture a second image of signal as a function of quality diagnostic.

Still referring to FIG. 1, in some embodiments, system 100 may display agent output 168. In some embodiments, system 100 may display agent output 168 using user interface 124. In some embodiments, first agent output may include converting agent output 168 to audio output data and outputting the audio output data using user interface 124. This may be performed using, for example, a text to speech system. In some embodiments, agent output 168 may be modified prior to being output, such as by converting agent output 168 to a natural language response 176 and displaying natural language response 176 using user interface 124. In some embodiments, natural language response 176 may be generated using second language model 180 based on agent output 168. Language models are described above.

Still referring to FIG. 1, in some embodiments, a visual element data structure may include a visual element. As used herein, a “visual element” is a datum that is displayed visually to a user. In some embodiments, a visual element data structure may include a rule for displaying visual element. In some embodiments, a visual element data structure may be determined as a function of agent output 168 and/or natural language response 176. In some embodiments, a visual element data structure may be determined as a function of an item from the list consisting of user input 128, multimodal data 132, procedure data 136, electronic health record data 140, one or more agents of plurality of agents 172, agent output 168, and/or natural language response 176. In a non-limiting example, a visual element data structure may be generated such that visual element describing or highlighting agent output 168 and/or natural language response 176 is displayed to a user. In a non-limiting example, a visual element may include an electronic health record including medical test results of a subject.

Still referring to FIG. 1, in some embodiments, visual element may include one or more elements of text, images, shapes, charts, particle effects, interactable features, and the like. In a non-limiting example, system 100 may include an element which a user may interact with in order to provide feedback to system 100 on a relevance of an output.

Still referring to FIG. 1, a visual element data structure may include rules governing if or when visual element is displayed. In a non-limiting example, a visual element data structure may include a rule causing a visual element describing agent output 168 and/or natural language response 176 to be displayed when a user selects agent output 168 and/or natural language response 176 using a graphical user interface (GUI).

Still referring to FIG. 1, a visual element data structure may include rules for presenting more than one visual element, or more than one visual element at a time. In an embodiment, about 1, 2, 3, 4, 5, 10, 20, or 50 visual elements are displayed simultaneously.

Still referring to FIG. 1, a visual element data structure rule may apply to a single visual element or datum, or to more than one visual element or datum. For example, a visual element data structure may rank visual elements and/or other data and/or apply numerical values to them, and a computing device may display a visual element as a function of such rankings and/or numerical values. A visual element data structure may apply rules based on a comparison between such a ranking or numerical value and a threshold.

Still referring to FIG. 1, in some embodiments, visual element may be interacted with. For example, visual element may include an interface, such as a button or menu. In some embodiments, visual element may be interacted with using a user device such as a smartphone.

Still referring to FIG. 1, in some embodiments, system 100 may transmit visual element to a display. A display may communicate visual element to user. A display may include, for example, a smartphone screen, a computer screen, or a tablet screen. A display may be configured to provide a visual interface. A visual interface may include one or more virtual interactive elements such as, without limitation, buttons, menus, and the like. A display may include one or more physical interactive elements, such as buttons, a computer mouse, or a touchscreen, that allow user to input data into the display. Interactive elements may be configured to enable interaction between a user and a computing device. In some embodiments, a visual element data structure is determined as a function of data input by user into a display.

Still referring to FIG. 1, in some embodiments, a system and/or method described herein may improve a process by which a medical professional may receive data relevant to an ongoing medical procedure. For example, input using voice commands may allow a user to receive data while occupied with another task. In another example, an ability to call multiple agents may improve versatility of system 100. In some embodiments, use of a language model selection of an agent may allow inputs to be received in a manner not dependent on specific language and/or commands, but using a more natural form of speech.

With continued reference to FIG. 1, in an embodiment, the agent orchestrator may be further configured to apply multiple agents in parallel, wherein applying the multiple agents in parallel may include processing, using the multiple agents, the first user input and procedure data concurrently, generating, using the multiple agents, a respective agent output for each agent of the multiple agents based on its specialized processing capabilities, and combining, using an aggregation function, the respective agent outputs of the multiple agents wherein the aggregation function comprises one or more of weighted averaging, consensus-based selection, or hierarchical ranking of the respective agent output. As used in this disclosure, “multiple agents” refers to two or more distinct computational entities, software modules, or AI-driven processes that are configured to perform specialized tasks independently or collaboratively within a system. Multiple agents may include, without limitation, rule-based agents, machine learning models, large language models (LLMs), expert systems, or any other autonomous or semi-autonomous components that process data, generate outputs, and contribute to decision-making. In an embodiment, multiple agents may operate concurrently, sequentially, or in a hierarchical manner to optimize task execution. Multiple agents may communicate, share information, or aggregate results to enhance accuracy, efficiency, or adaptability based on system requirements. As used in this disclosure, “a respective agent output” is an output generated by an individual agent in response to processing input data. A respective agent output may include, without limitation, a prediction, classification, recommendation, data transformation, report, decision, or any other computational result produced by the agent. In an embodiment, each respective agent output may be based on the specialized processing capabilities of each of the multiple agents, underlying algorithms, or learned models. The respective agent output may be used independently, combined with outputs from other agents, or further processed by an aggregation function to generate a final response within a multi-agent system. As used in this disclosure, “an aggregation function” is a computational process configured to synthesize multiple inputs into a single output. The aggregation function may include, without limitation, statistical methods, machine learning models, consensus-based algorithms, rule-based logic, and the like to process data from multiple sources. In some embodiments, the aggregation function may operate on outputs from multiple agents to generate a unified result, wherein the aggregation function comprises one or more of weighted averaging, majority voting, hierarchical ranking, or confidence-based selection. The aggregation function may be dynamically adapted based on task requirements, input variability, or system constraints to optimize decision-making and response accuracy. As used in this disclosure, “weighted averaging” is an aggregation function that combines multiple inputs by assigning different weights to each input based on predefined criteria. In an embodiment, the predefined criteria may include confidence scores, historical accuracy, source reliability, and the like. The final output is computed as a weighted sum or mean of the inputs, ensuring that higher-weighted contributions have a greater influence on the result. As used in this disclosure, “consensus-based selection” is an aggregation function that determines the final output by identifying the most agreed-upon result among multiple inputs. Without limitation, the consensus-based selection may include majority voting, statistical mode calculation, agreement thresholds, and the like to prioritize outputs that are most frequently selected or aligned among contributing agents. As used in this disclosure, “hierarchical ranking” is an aggregation function that prioritizes and organizes multiple inputs based on a predefined hierarchy of importance, relevance, or confidence. The highest-ranked input is selected as the final output, or a combination of top-ranked inputs may be used to generate a refined result. Hierarchical ranking may be dynamically adjusted based on context, user preferences, or system-defined parameters.

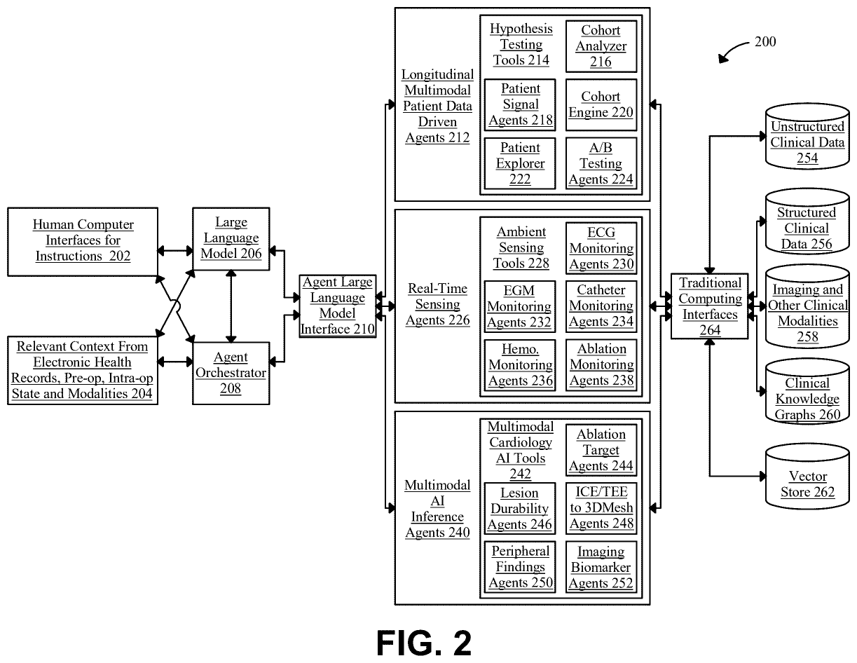

Referring now to FIG. 2, an exemplary embodiment of a system 200 including an agent orchestrator is provided. System 200 may include one or more human computer interfaces 202. Such human computer interfaces may be used to receive instructions for system 200. System 200 may receive relevant context 204 from electronic health records, pre-op, intra-op state and modalities. Instructions and/or context may be input into large language model 206 and/or agent orchestrator 208, which may interact with agents using agent large language model interface 210.

Still referring to FIG. 2, in some embodiments, system 200 may include longitudinal multimodal patient data driven agents 212, which may include hypothesis testing tools 214 such as cohort analyzer 216, patient signal agents 218, cohort engine 220, patient explorer 222, and/or A/B testing agents 224. In some embodiments, system 200 may include real-time sensing agents 226 which may include ambient sensing tools 228 such as ECG monitoring agents 230, EGM monitoring agents 232, catheter monitoring agents 234, hemodynamic monitoring agents 236, and/or ablation monitoring agents 238. In some embodiments, system 200 may include multimodal AI inference agents 240 which may include multimodal cardiology AI tools 242 such as ablation target agents 244, lesion durability 246, ICE/TEE to 3DMesh agents 248, peripheral findings agents 250, and/or imaging biomarker agents 252.

Still referring to FIG. 2, in some embodiments, one or more agents may interact with and/or receive unstructured clinical data 254, structured clinical data 256, imaging and other clinical modalities 258, clinical knowledge graphs 260 and/or vector store 262 using traditional computing interfaces 264.

Now referring to FIG. 3, a flow diagram of an exemplary embodiment of an ICE example generation process 300. In an embodiment, cardiac anatomy training data may be generated, at least in part, via ICE example generation process 300. In some cases, processor 104 may be configured to receive a 3D model of the heart, such as any 3D model of cardiac anatomy 312 as described herein and identify an ICE view 304 (i.e., visual representation of image obtained using intracardiac echocardiography as described above e.g., ICE image) based on the received 3D model. In some cases, 3D model received by processor 104 may be derived from CT scans as described above with reference to FIG. 1. In other cases, processor may receive CT scans directly instead of 3D models. A synthetic ICE frame 308 may then be generated, by processor 104, as a function of identified ICE view 304, wherein the synthetic ICE frame 308 may be used as one or the training examples in cardiac anatomy training data.

With continued reference to FIG. 3, in some cases, processor 104 may interface with one or more 3D models (i.e., detailed representation of heart's anatomy in a 3D space, capturing intricate structures, chambers, vessels, valves, among others) as described above, or other imaging modalities and/or databases, and equipped with algorithms e.g., CNN, gradient boosting machines, SVM, PCA, and/or the like to analyze model's geometry and spatial relationships upon receiving the 3D models. In some cases, 3D models may be received from SSM 316 as described above with reference to FIG. 1 via a communicative connection between processor 104 and SSM 316. In a non-limiting example, processor 104 may be configured to determine an optimal viewpoints or angles from which ICE view 304 would provide a desired diagnostic value or procedural guidance.

Still referring to FIG. 3, in some cases, identification and selection of ICE view 304 may be automatically identified, using one or more machine learning models as described herein. In a non-limiting example, processor 104 may utilize one or more machine learning models trained on cardiac anatomy viewpoints identification training data, wherein the cardiac anatomy viewpoints identification training data may include a plurality of cardiac anatomies as input correlated to a plurality of ICE images as output and identify at least one ICE view 304 (most informative) for a given cardiac anatomy using the trained machine learning models.

Still referring to FIG. 3, in other cases, ICE view 304 may be defined by a user such as a medical professional. In a non-limiting example user interface of display device may allow a user (e.g., a clinician) to manually rotate, pan, and zoom displayed 3D model and/or corresponding CT scans. As user do so, processor 104 may dynamically calculate and displays potential ICE views 304 based on user's chosen perspective. Additionally, or alternatively, depending on cardiac procedure being planned or executed, processor 104 may prioritize certain ICE views 304. For instance, and without limitation, ICE view 304 may be pre-defined. For atrial fibrillation ablation, ICE view 304 may showcase the pulmonary veins' entrances into the LA may be emphasized. In other cases, ICE view 304 may be automatically identified, by processor 104, using one or more machine learning models as described herein, such as, without limitation, synthetic ICE data generator as described in detail below.

With continued reference to FIG. 3, as used in this disclosure, a “synthetic ICE frame” refers to a digitally generated or simulated image that emulates a visual representation obtained from ICE view 304. In some cases, synthetic ICE frames 308 may be produced using computational methods and/or models such as, without limitation, a synthetic ICE data generator based on pre-existing data, models, or simulations e.g., identified ICE views 304. In a non-limiting example, synthetic ICE frames 308 may include a simplified version e.g., an image illustrating heart anatomy via a plurality of lines indicating contours of heart's structure as shown in FIG. 3. One or more image processing techniques and/or computer vision algorithms such as, without limitation, histogram equalization, adaptive filtering, edge detection (e.g., Canny or Sobel operators), contour extraction, and/or the like may be applied, by processor 104, on a segmented CT scan and/or 3D models based on identified ICE view 304. Synthetic ICE frame 308 may be rendered on a blank canvas or background that mimics the echogenicity of an ICE image according to extracted contours, wherein the extracted contours may be represented as a bold lines and enhanced with shading to give depth. In some cases, synthetic ICE frame 308 may be validated and verified by overlaying synthetic ICE frame 308 onto original ICE view 304, ensuring accuracy and resemblance.

Still referring to FIG. 3, in some cases, generating synthetic ICE frames 308 may include implementations of one or more aspects of “generative artificial intelligence,” a type of AI that uses machine learning algorithms to create, establish, or otherwise generate data such as, without limitation, ICE images, ICE videos, and/or the like that is similar to one or more provided training examples. In an embodiment, machine learning module described herein may generate one or more generative machine learning models that are trained on one or more set of CT scans and/or 3D models in ICE image view 304 as described above. Synthetic ICE data generator may include one or more generative machine learning models may be configured to generate new examples that are similar to the training data of the one or more generative machine learning models but are not exact replicas; for instance, and without limitation, data quality or attributes of the generated examples may bear a resemblance to the training data provided to one or more generative machine learning models, wherein the resemblance may pertain to underlying patterns, features, or structures found within the provided training data.

Still referring to FIG. 3, in some cases, generative machine learning models within synthetic ICE data generator may include one or more generative models. As described herein, “generative models” refers to statistical models of the joint probability distribution P(X, Y) on a given observable variable x, representing features or data that can be directly measured or observed (e.g. CT scans and/or 3D models derived from CT scans) and target variable y, representing the outcomes or labels that one or more generative models aims to predict or generate (e.g., synthetic ICE frames 308). In some cases, generative models may rely on Bayes theorem to find joint probability; for instance, and without limitation, Naïve Bayes classifiers may be employed by computing device to categorize input data such as, without limitation, CT scans and/or 3D models derived from CT scans into different views.

In a non-limiting example, and still referring to FIG. 3, one or more generative machine learning models may include one or more Naïve Bayes classifiers generated, by processor 104, using a Naïve bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Processor 104 may then calculate a likelihood table by calculating probabilities of different data entries and classification labels. Processor 104 may utilize a naïve Bayes equation to calculate a posterior probability for each class. A class containing the highest posterior probability is the outcome of prediction.

Still referring to FIG. 3, although Naïve Bayes classifier may be primarily known as a probabilistic classification algorithm; however, it may also be considered a generative model described herein due to its capability of modeling the joint probability distribution P(X, Y) over observable variables X and target variable Y. In an embodiment, Naïve Bayes classifier may be configured to make an assumption that the features X are conditionally independent given class label Y, allowing generative model to estimate the joint distribution as P(X, Y)=P(Y)ΠiP(Xi|Y), wherein P(Y) may be the prior probability of the class, and P(Xᵢ|Y) is the conditional probability of each feature given the class. One or more generative machine learning models containing Naïve Bayes classifiers may be trained on labeled training data, estimating conditional probabilities P(Xᵢ|Y) and prior probabilities P(Y) for each class; for instance, and without limitation, using techniques such as Maximum Likelihood Estimation (MLE). One or more generative machine learning models containing Naïve Bayes classifiers may select a class label y according to prior distribution P(Y), and for each feature Xᵢ, sample at least a value according to conditional distribution P(Xᵢ|y). Sampled feature values may then be combined to form one or more new data instance with selected class label y. In a non-limiting example, one or more generative machine learning models may include one or more Naïve Bayes classifiers to generate new examples of ICE images based on CT scans and/or 3D models derived from CT scans (e.g., identified ICE views 304), wherein the models may be trained using training data containing a plurality of features of input data as described herein and/or the like correlated to a plurality of ICE views.

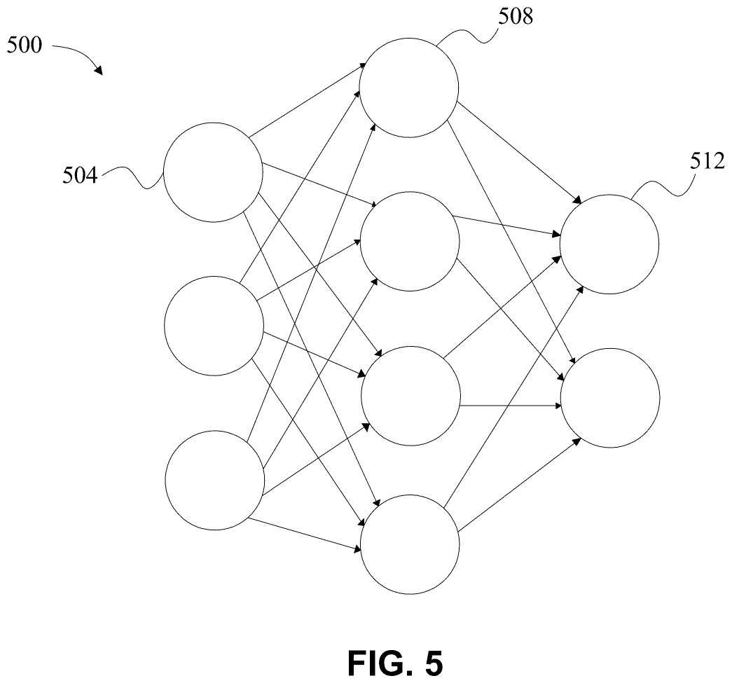

Still referring to FIG. 3, in some cases, one or more generative machine learning models may include generative adversarial network (GAN). As used in this disclosure, a “generative adversarial network” is a type of artificial neural network with at least two sub models (e.g., neural networks), a generator, and a discriminator, that compete against each other in a process that ultimately results in the generator learning to generate new data samples, wherein the “generator” is a component of the GAN that learns to create hypothetical data by incorporating feedbacks from the “discriminator” configured to distinguish real data from the hypothetical data. In some cases, generator may learn to make discriminator classify its output as real. In an embodiment, discriminator may include a supervised machine learning model while generator may include an unsupervised machine learning model as described in further detail with reference to FIGS. 5-7.

With continued reference to FIG. 3, in an embodiment, discriminator may include one or more discriminative models, i.e., models of conditional probability P(Y|X=x) of target variable Y, given observed variable X. In an embodiment, discriminative models may learn boundaries between classes or labels in given training data. In a non-limiting example, discriminator may include one or more classifiers as described in further detail below with reference to FIG. 5 to distinguish between different categories e.g., real vs. fake, or states e.g., TRUE vs. FALSE within the context of generated data such as, without limitations, synthetic ICE frames 308, and/or the like. In some cases, processor 104 may implement one or more classification algorithms such as, without limitation, Support Vector Machines (SVM), Logistic Regression, Decision Trees, and/or the like to define decision boundaries.

In a non-limiting example, and still referring to FIG. 3, generator of GAN may be responsible for creating synthetic data that resembles real ICE images. In some cases, GAN may be configured to receive CT scans and/or 3D models derived from CT scans as input and generates corresponding examples of ICE images containing information describing heart anatomy in different ICE views. On the other hand, discriminator of GAN may evaluate the authenticity of the generated content by comparing it to true ICE images, for example, discriminator may distinguish between genuine and generated content and providing feedback to generator to improve the model performance. Additionally, or alternatively, GAN may include a conditional GAN as an extension of the basic GAN as described herein that allows for generation of ICE images using pre-existing CT scans and/or 3D models derived from CT scans based on certain conditions or labels. In standard GAN, generator may produce samples from random noise, while in a conditional GAN, generator may produce samples based on random noise and a given condition or label.

With continued reference to FIG. 3, in other embodiments, one or more generative models may also include a variational autoencoder (VAE). As used in this disclosure, a “variational autoencoder” is an autoencoder (i.e., an artificial neural network architecture) whose encoding distribution is regularized during the model training process in order to ensure that its latent space includes desired properties allowing new data sample generation. In an embodiment, VAE may include a prior and noise distribution respectively, trained using expectation-maximization meta-algorithms such as, without limitation, probabilistic PCA, sparse coding, among others. In a non-limiting example, VEA may use a neural network as an amortized approach to jointly optimize across input data and output a plurality of parameters for corresponding variational distribution as it maps from a known input space to a low-dimensional latent space. Additionally, or alternatively, VAE may include a second neural network, for example, and without limitation, a decoder, wherein the “decoder” is configured to map from the latent space to the input space.

In a non-limiting example, and still referring to FIG. 3, VAE may be used by processor 104 to model complex relationships between CT scans and/or 3D models derived from CT scans. In some cases, VAE may encode input data into a latent space, capturing example ICE images. Such encoding process may include learning one or more probabilistic mappings from observed CT scans and/or 3D models derived from CT scans to a lower-dimensional latent representation. Latent representation may then be decoded back into the original data space, therefore reconstructing the 3D models representing example ICE images. In some cases, such decoding process may allow VAE to generate new examples or variations that are consistent with the learned distributions.

Additionally, or alternatively, and still referring to FIG. 3, processor 104 may be configured to continuously monitor synthetic ICE data generator. In an embodiment, processor 104 may configure discriminator to provide ongoing feedback and further corrections as needed to subsequent input data. An iterative feedback loop may be created as processor 104 continuously receive real-time data, identify errors (e.g., distance between synthetic ICE frame 308 and real ICE images) as a function of real-time data, delivering corrections based on the identified errors, and monitoring subsequent model outputs and/or user feedbacks on the delivered corrections. In an embodiment, processor 104 may be configured to retrain one or more generative machine learning models within synthetic ICE data generator based on user modified ICE frames or update training data of one or more generative machine learning models within synthetic ICE data generator by integrating validated synthetic ICE frames (i.e., subsequent model output) into the original training data. In such embodiment, iterative feedback loop may allow synthetic ICE data generator to adapt to the user's needs and performance requirements, enabling one or more generative machine learning models described herein to learn and update based on user responses and generated feedbacks.

With continued reference to FIG. 3, other exemplary embodiments of generative machine learning models may include, without limitation, long short-term memory networks (LSTMs), (generative pre-trained) transformer (GPT) models, mixture density networks (MDN), and/or the like. As an ordinary person skilled in the art, upon reviewing the entirety of this disclosure, will be aware of various generative machine learning models that may be used generating synthetic ICE frames 308.

Still referring to FIG. 3, in a further non-limiting embodiment, synthetic ICE data generator may be further configured to generate a multi-model neural network that combines various neural network architectures described herein. In a non-limiting example, multi-model neural network may combine LSTM for time-series analysis with GPT models for natural language processing. Such fusion may be applied by computing device to generate synthetic ICE frames 308. In some cases, multi-model neural network may also include a hierarchical multi-model neural network, wherein the hierarchical multi-model neural network may involve a plurality of layers of integration; for instance, and without limitation, different models may be combined at various stages of the network. Convolutional neural network (CNN) may be used for image feature extraction, followed by LSTMs for sequential pattern recognition, and a MDN at the end for probabilistic modeling. Other exemplary embodiments of multi-model neural network may include, without limitation, ensemble-based multi-model neural network, cross-modal fusion, adaptive multi-model network, among others. As an ordinary person skilled in the art, upon reviewing the entirety of this disclosure, will be aware of various generative machine learning models that may be used to generating synthetic ICE frames 308 as described herein. As an ordinary person skilled in the art, upon reviewing the entirety of this disclosure, will be aware of various multi-model neural network and combination thereof that may be implemented in consistent with this disclosure.

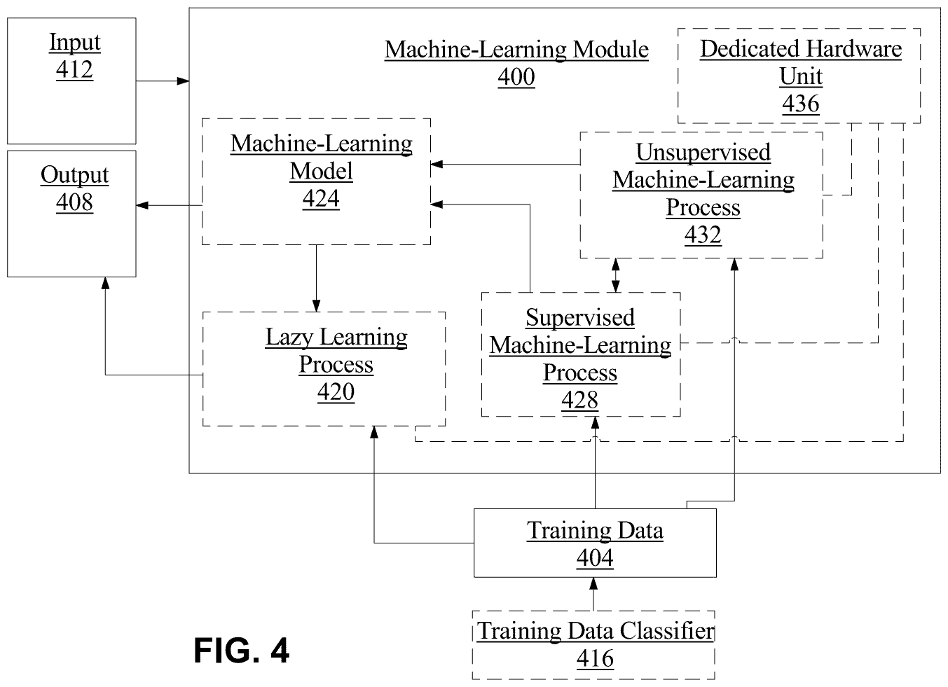

Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm instantiated in hardware or software logic, data structures, and/or functions that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example, inputs may include user inputs and outputs may include an agent selection datum.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data to particular medical procedures.

Still referring to FIG. 4, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 4, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 4, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{Σ}_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 4, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 4, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 4, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 4, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 4, computing device, processor, and/or module may be configured to precondition one or more training examples. For instance, and without limitation, where α machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 4, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 4, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 4, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{median}}{IQR} .$$

Persons skilled in the art, upon reviewing the entirety of this disclosure, will be aware of various alternative or additional approaches that may be used for feature scaling.

Further referring to FIG. 4, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 4, machine-learning processes as described in this disclosure may be used to generate machine-learning models 424. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include user inputs as described above as inputs, agent selection data as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

With further reference to FIG. 4, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where α “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 4, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 432 may not require a response variable; unsupervised processes 432 may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 4, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 4, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 4, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 4, one or more processes or algorithms described above may be performed by at least a dedicated hardware unit 436. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 436 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 436 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 436 to perform one or more operations described herein, such as evaluation of model and/or algorithm outputs, one-time or iterative updates to parameters, coefficients, weights, and/or biases, and/or any other operations such as vector and/or matrix operations as described in this disclosure.

With continued reference to FIG. 4, system 100 may use user feedback to train the machine-learning models and/or classifiers described above. For example, classifier may be trained using past inputs and outputs of classifier. In some embodiments, if user feedback indicates that an output of classifier was “bad,” then that output and the corresponding input may be removed from training data used to train classifier, and/or may be replaced with a value entered by, e.g., another user that represents an ideal output given the input the classifier originally received, permitting use in retraining, and adding to training data; in either case, classifier may be retrained with modified training data as described in further detail below. In some embodiments, training data of classifier may include user feedback.

With continued reference to FIG. 4, in some embodiments, an accuracy score may be calculated for classifier using user feedback. For the purposes of this disclosure, “accuracy score,” is a numerical value concerning the accuracy of a machine-learning model. For example, a plurality of user feedback scores may be averaged to determine an accuracy score. In some embodiments, a cohort accuracy score may be determined for particular cohorts of persons. For example, user feedback for users belonging to a particular cohort of persons may be averaged together to determine the cohort accuracy score for that particular cohort of persons and used as described above. Accuracy score or another score as described above may indicate a degree of retraining needed for a machine-learning model such as a classifier; system 100 may perform a larger number of retraining cycles for a higher number (or lower number, depending on a numerical interpretation used), and/or may collect more training data for such retraining, perform more training cycles, apply a more stringent convergence test such as a test requiring a lower mean squared error, and/or indicate to a user and/or operator that additional training data is needed.

Referring now to FIG. 5, an exemplary embodiment of neural network 500 is illustrated. A neural network 500 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 504, one or more intermediate layers 508, and an output layer of nodes 512. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes.

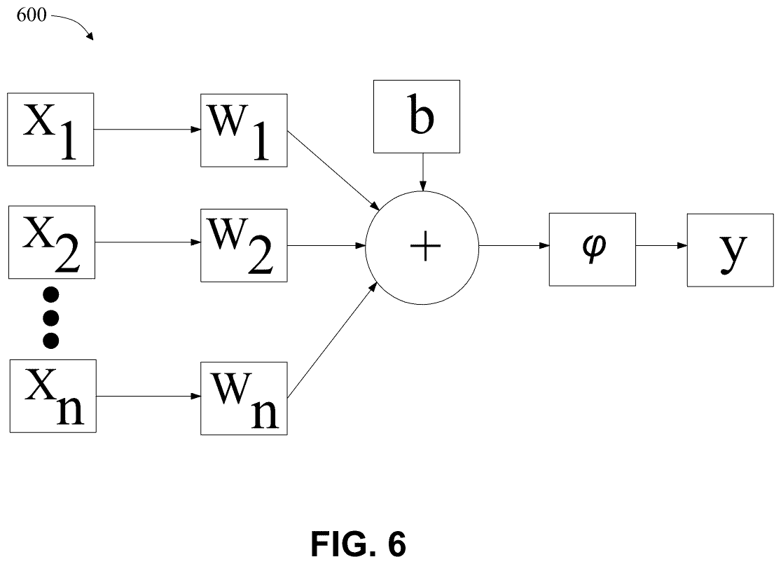

Referring now to FIG. 6, an exemplary embodiment of a node 600 of a neural network is illustrated. A node may include, without limitation a plurality of inputs xi that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tan h (hyperbolic tangent) function, of the form

$$\frac{{e}^{x} - {e}^{- x}}{{e}^{x} + {e}^{- x}} ,$$

a tan h derivative function such as ƒ(x)=tan h²(x), a rectified linear unit function such as ƒ(x)=max(0, x), a “leaky” and/or “parametric” rectified linear unit function such as ƒ(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{Σ}_{i} {x}_{i}}$$

where the inputs to an instant layer are xᵢ, a swish function such as ƒ(x)=x*sigmoid (x), a Gaussian error linear unit function such as f(x)=a(1+tan h(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs x; that may be used as activation functions. As a non-limiting and illustrative example, node may perform a weighted sum of inputs using weights w; that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights w; may be determined by training a neural network using training data, which may be performed using any suitable process as described above.

Still referring to FIG. 6, a “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like. CNN may include, without limitation, a deep neural network (DNN) extension, where a DNN is defined as a neural network with two or more hidden layers.

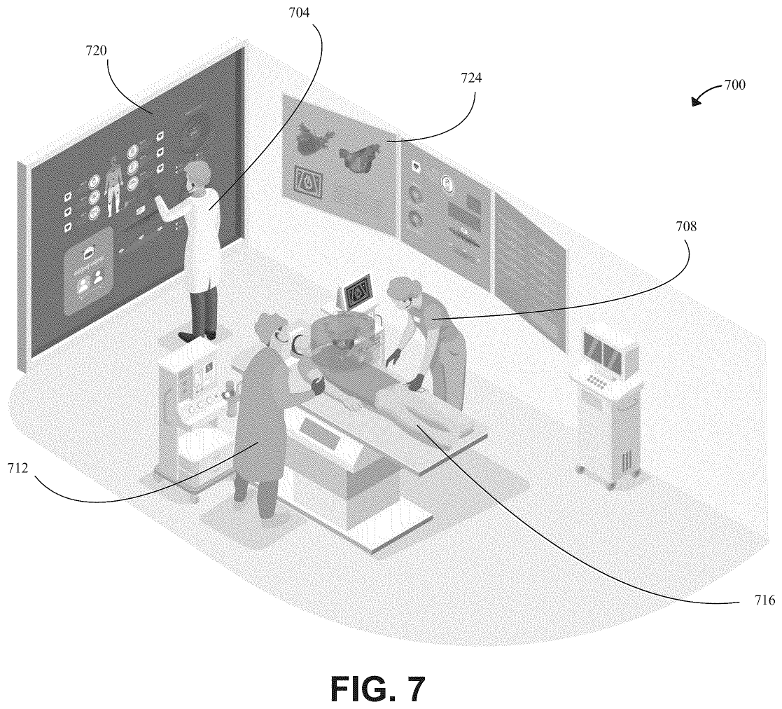

Referring now to FIG. 7, a representation of an exemplary operatory 700 during an electrophysiology procedure using an exemplary stylized electrophysiology copilot is shown. In some embodiments, operatory 700 may include one or more users such as users 704, 708, and 712, which may operate a system such as an electrophysiology copilot described herein. Operatory 700 may further include subject 716. Operatory 700 may further include one or more interfaces, such as interfaces 720 and 724. Such interfaces may, in non-limiting examples, display procedure data as described herein and/or allow users to input data into electrophysiology copilot. In some embodiments, an electrophysiology copilot may be consistent with a system described above, such as a system including an agent orchestrator and one or more agents. An electrophysiology copilot may include an agentic artificial intelligence (AI) system that implements a co-pilot for operators, physicians, mappers and/or technicians involved in an electrophysiology procedure (EP). In some embodiments, tasks commonly performed today by technicians and mappers, for instance at voice instruction by a physician, may be performed by an agentic AI system. An agentic AI system may provide specific functionality such as, in non-limiting examples, patient specific information retrieval germane to EP procedure, either as an unprompted recommendation or based on a voice, text, or touch-based command. In some cases, a user, via an agentic AI system may pose clinical queries and/or hypotheses to a real world database, including, for example, a database of details of prior EP procedures. In some embodiments, an agentic AI system may, again as an unprompted recommendation or based on a user command, present specific views to physician. Agentic AI may, without limitation, make specific recommendations that are clinical, to be considered intra-operatively, based on patient, cohort, and/or context. Agentic AI may recommend a specific motion along specific paths for any of a number of catheters. In some embodiments, agentic AI may be in communication (e.g., real-time communication) with (1) devices configured for ambient sensing of events; (2) sensors attached to catheters; and/or (3) other medical devices. In some cases, based upon external and/or internal signals, agentic AI may trigger notifications, alarms, or other specific actions, including but not restricted to (1) automatically displaying specific imaging views, angles, or perspectives, with or without appropriate voiceovers, as required; and (2) autonomous control sequence of one or more specific EP devices, e.g. catheters.

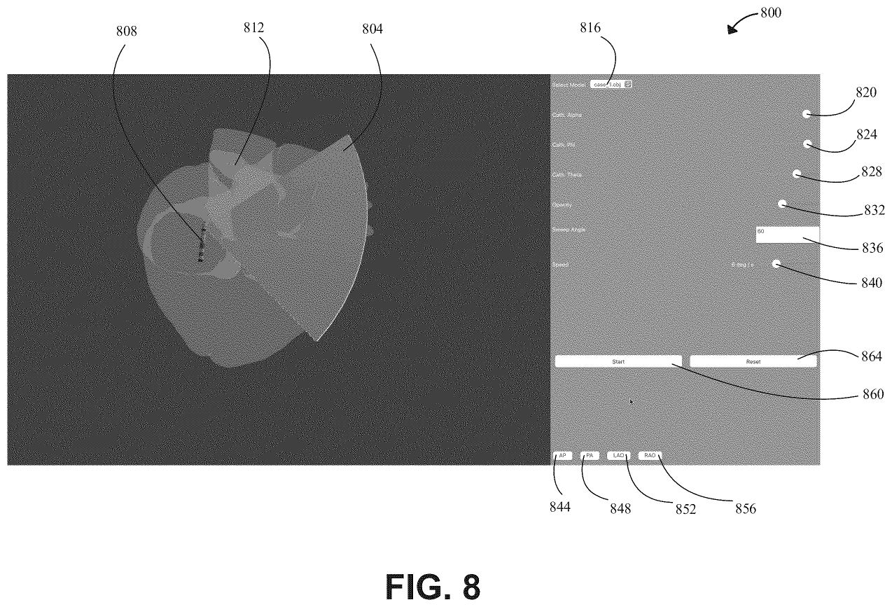

Referring now to FIG. 8, an illustration of an exemplary interface 800 for an electrophysiology copilot is provided. In some embodiments, electrophysiology copilot may depict a position of a catheter within an organ such as a heart. In some embodiments, electrophysiology copilot may depict a field of view 804 of a sensor of a catheter 808, e.g. an intracardiac echocardiogram (ICE) or transesophageal echocardiogram (TEE) ultrasonic transducer, with respect to a modeled organ 812. In some embodiments, electrophysiology copilot may include functionality for selecting one or more models, such as through use of interactable element 816. In some embodiments, electrophysiology copilot may include one or more settings for viewing a model, such as Catheter Alpha 820, Catheter Phi 824, and/or Catheter Theta 828, which may control an orientation of a catheter. In some embodiments, interface 800 may further include an opacity setting 832, which may control an opacity of modeled organ 812. In some embodiments, electrophysiology copilot may be used to view a field of view as it sweeps around a catheter. Non-limiting examples of settings which may be adjusted for viewing a moving field of view include sweep angle 836 and speed 840. In some embodiments, a depicted field of view may include a 2D slice or cross-section of a 3D model. In some embodiments, interface 800 may further include one or more interactable elements, such as interactable elements 844, 848, 852, and 856, which position and/or orient a perspective to a predetermined position with respect to a modeled organ, such as a left anterior oblique (LAO) view, a right anterior oblique (RAO), an anteroposterior (AP), or a posteroanterior (PA) position. Interface 800 may further include an interactable element 860 which starts movement of a field of view with respect to a 3D model, and/or an interactable element 864 which resets a field of view with respect to a 3D model.

Still referring to FIG. 8, electrophysiology copilot may generate 3D mesh from ICE-based 3D or a cardiac computed tomography (CT). In some embodiments, a user such as a physician or technician may use electrophysiology copilot to do one or more of the following actions, one or more times: (a) Specify using an input such as a mouse, touch, voice, keyboard, text, or other input a trajectory to be followed by an ICE catheter to create a sequence of “ICE views.” Such ICE views may include, or be used to derive, metrics such as frame rates, angles between each frame, overall sweep angle, and the like. In some embodiments, ICE views may have corresponding “synthetic ICE frames” which may be used for downstream training of an ICE to 3d system. (b) Train junior physicians or technicians on what trajectories are to be taken by the ICE catheter to capture good views of specific structures in specific chambers. Or (c) train junior physicians or technicians on what trajectories are to be taken by the ICE catheter to capture good views of specific structures in specific chambers, in response to an ICE video the junior physician has been shown and asked to reproduce. In some embodiments, an electrophysiology copilot may be used for similar training with other catheter types. In some embodiments, an electrophysiology copilot may be operated through use of voice based commands. In a non-limiting example, a system may respond to a user voice command such as: “show me the view if a camera were to be placed on the coronary sinus, facing towards the left atrial appendage (LAA).”

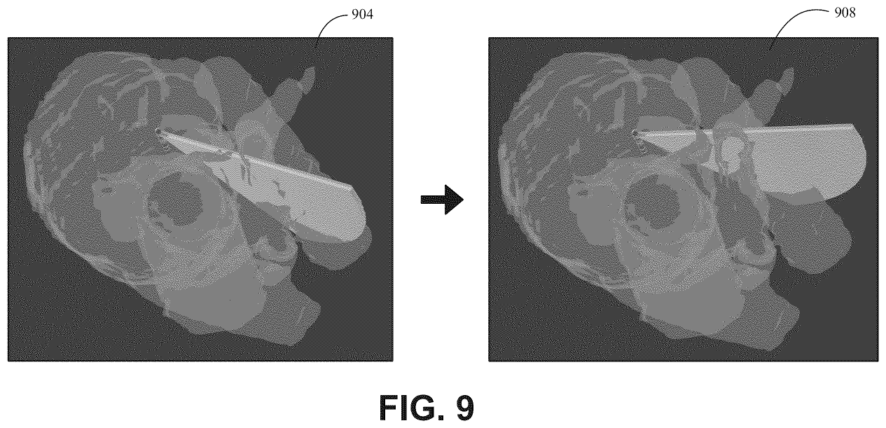

Referring now to FIG. 9, two illustrations of exemplary embodiments of an interface of an electrophysiology copilot are provided, while a catheter sensor is swept within a heart. In some embodiments, an interface may depict a field of view of a sensor on a catheter rotating around a catheter. For example, a catheter may remain translationally stationary (aside from rotating) and a field of view may rotate (i.e., sweep) from a position depicted in image 904 to a position depicted in image 908. In some embodiments, a sensor depicted using an electrophysiology copilot interface may include, in non-limiting examples, an ICE or TEE ultrasound transducer. In some embodiments, a field of view sweep may be performed for 3D model construction and/or catheter localization.

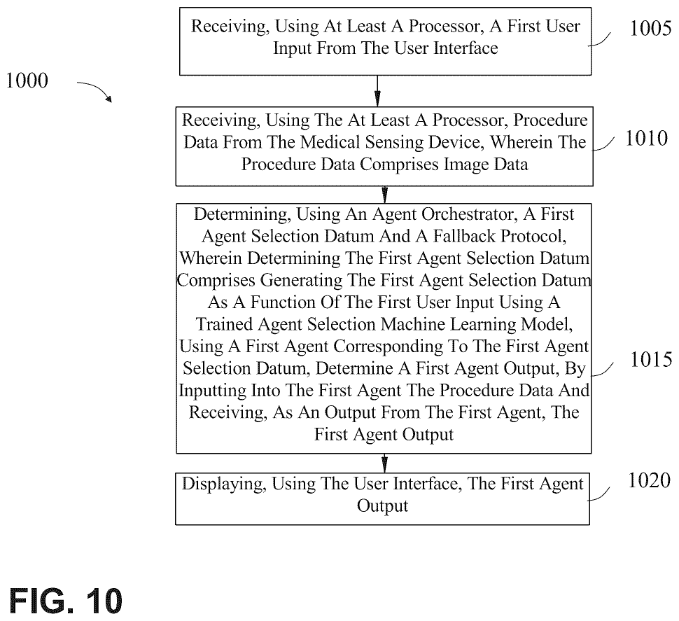

Referring now to FIG. 10, an exemplary embodiment of a method 1000 of responding to a user input using an agent orchestrator is illustrated. One or more steps if method 1000 may be implemented, without limitation, as described with reference to other figures. One or more steps of method 1000 may be implemented, without limitation, using at least a processor. This may be implemented as described and with reference to FIGS. 1-9.

Still referring to FIG. 10, in some embodiments, method 1000 may include a step 1005 of receiving, using at least a processor, a first user input from the user interface. This may be implemented as described and with reference to FIGS. 1-9.

Still referring to FIG. 10, in some embodiments, method 1000 may include a step 1010 of receiving, using the at least a processor, procedure data from the medical sensing device, wherein the procedure data comprises image data. This may be implemented as described and with reference to FIGS. 1-9.

Still referring to FIG. 10, in some embodiments, method 1000 may include a step 1015 of determining, using an agent orchestrator, a first agent selection datum and a fallback protocol, wherein determining the first agent selection datum comprises generating the first agent selection datum as a function of the first user input using a trained agent selection machine learning model, using a first agent corresponding to the first agent selection datum, determine a first agent output, by inputting into the first agent the procedure data and receiving, as an output from the first agent, the first agent output. In some embodiments, step 1015 may include generating the first agent selection datum as a function of the first user input using a trained agent selection machine learning model. This may be implemented as described and with reference to FIGS. 1-9.

Still referring to FIG. 10, in some embodiments, method 1000 may include a step 1020 of displaying, using the user interface, the first agent output. In some embodiments, step 1020 may include inputting into the first agent the procedure data and receiving, as an output from the first agent, the first agent output. In some embodiments, the procedure data includes ultrasonic image data. In some embodiments, the first agent is configured to generate a set of shape parameters representing a structure's shape as a function of the ultrasonic image data and a shape identification model trained on a training dataset includes historical ultrasonic images correlated with historical computed tomography scan data and generate a 3D model of the structure based on the set of shape parameters, wherein the first agent output includes the 3D model. In some embodiments, the procedure data includes a Pulsed Field Ablation (PFA) device parameter and determining the first agent output includes generating a PFA durability datum as a function of the PFA device parameter using a trained PFA durability machine learning model. In some embodiments, the procedure data includes a Pulsed Field Ablation (PFA) device parameter, and the first agent includes a lesion durability agent configured to generate a PFA durability datum as a function of the PFA device parameter using a trained PFA durability machine learning model; and the first agent output includes the PFA durability datum. This may be implemented as described and with reference to FIGS. 1-9.

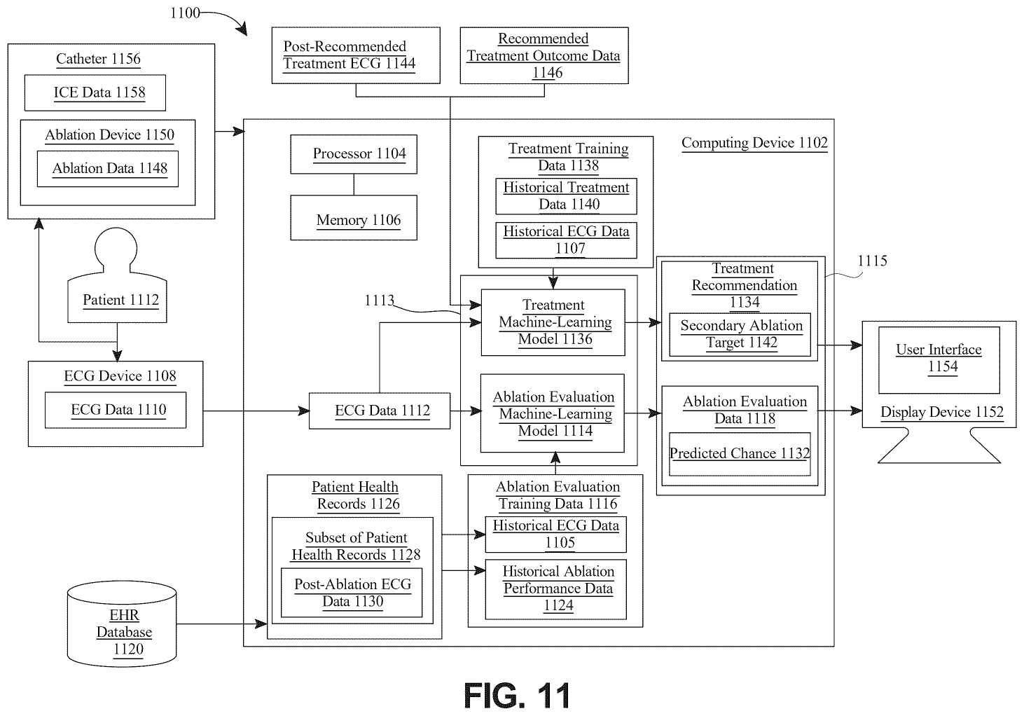

Referring now FIG. 11, an exemplary embodiment of an apparatus 1100 for prediction of repeat ablation efficacy is illustrated. Apparatus 1100 includes a computing device 1102. Computing device includes a processor 1104 communicatively connected to a memory 1106. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring FIG. 11, computing device 1102 may include any computing device as described in this disclosure, including without limitation a microcontroller, microprocessor, digital signal processor (DSP) and/or system on a chip (SoC) as described in this disclosure. computing device 1102 may include, be included in, and/or communicate with a mobile device such as a mobile telephone or smartphone. computing device 1102 may include a single computing device operating independently, or may include two or more computing device operating in concert, in parallel, sequentially or the like; two or more computing devices may be included together in a single computing device or in two or more computing devices. computing device 1102 may interface or communicate with one or more additional devices as described below in further detail via a network interface device. Network interface device may be utilized for connecting computing device 1102 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 1102 may include but is not limited to, for example, a computing device or cluster of computing devices in a first location and a second computing device or cluster of computing devices in a second location. Computing device 1102 may include one or more computing devices dedicated to data storage, security, distribution of traffic for load balancing, and the like. Computing device 1102 may distribute one or more computing tasks as described below across a plurality of computing devices of computing device, which may operate in parallel, in series, redundantly, or in any other manner used for distribution of tasks or memory between computing devices. computing device 1102 may be implemented, as a non-limiting example, using a “shared nothing” architecture.

With continued reference FIG. 11, computing device 1102 may be designed and/or configured to perform any method, method step, or sequence of method steps in any embodiment described in this disclosure, in any order and with any degree of repetition. For instance, computing device 1102 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. computing device 1102 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference FIG. 11, memory 1106 may include a primary memory and a secondary memory. “Primary memory” also known as “random access memory” (RAM) for the purposes of this disclosure is a short-term storage device in which information is processed. In one or more embodiments, during use of the computing device, instructions and/or information may be transmitted to primary memory wherein information may be processed. In one or more embodiments, information may only be populated within primary memory while a particular software is running. In one or more embodiments, information within primary memory is wiped and/or removed after the computing device has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 1104 may access the information from primary memory.

With continued reference FIG. 11, apparatus 1100 may include an electrocardiogram device 1108. An “electrocardiogram device,” for the purposes of this disclosure, is a device that records the electrical activity of a heart over time. In some embodiments, electrocardiogram (ECG) device 1108 may be configured to preform electrocardiogra

With continued reference FIG. 11, electrocardiogramedical procedure that records the electrical activity of the heart over time, producing an electrocardiogram (ECG or EKG). This process may include placing electrodes on the skin to detect small electrical changes resulting from cardiac muscle depolarization and repolarization during each heartbeat. The ECG may provide a graphical representation of voltage versus time, offering valuable insights into the heart's function and overall cardiac health.

With continued reference FIG. 11, ECGs may be crucial in identifying various cardiac abnormalities, including rhythm disturbances like atrial fibrillation and ventricular tachycardia, inadequate coronary artery blood flow conditions such as myocardial ischemia and infarction, and electrolyte imbalances like hypokalemia.

With continued reference FIG. 11, ECG device 1108 is configured to detect ECG data 1110 of a heart of a patient 1112. For the purposes of this disclosure, “ECG data” is data relating to the electrical activity of a heart over time. ECG data may include data from one or more electrodes in contact with the patient's limbs and/or chest. In some embodiments, ECG data may include 12-lead ECG data. For the purposes of this disclosure, “12-lead ECG data,” is ECG data that was collected from an electrocardiogram device having 12 leads. A 12-lead ECG may include placing ten electrodes on the patient's limbs and chest surface. This configuration allows the measurement of the heart's electrical potential from twelve different angles or “leads” over a period of typically ten seconds. By capturing the magnitude and direction of the heart's electrical depolarization throughout the cardiac cycle, the ECG may provide a comprehensive view of the heart's electrical activity, enabling healthcare professionals to assess cardiac function and diagnose potential issues.

With continued reference FIG. 11, in some embodiments, electrocardiogram device 1108 may be configured to detect post-ablation arrhythmic electrocardiogram data. For the purposes of this disclosure, “post-ablation arrhythmic electrocardiogram data” is data from an electrocardiogram conducted on a patient that suffers from a cardiac arrhythmia and has undergone an ablation procedure. In some embodiments, post-ablation arrhythmic electrocardiogram data may be representative of a post-ablation arrhythmia of a patient who has previously undergone an ablation procedure.

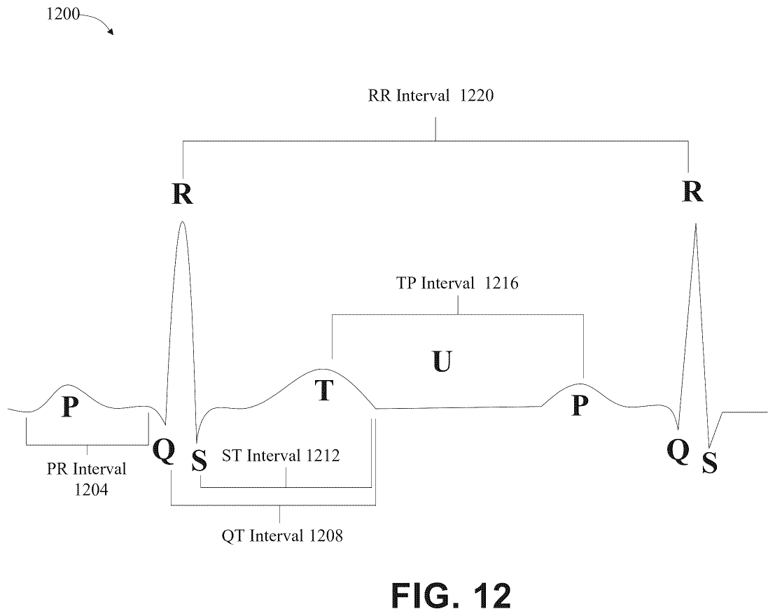

With continued reference FIG. 11, a standard embodiment of ECG is a 12-lead ECG, however additional embodiments with fewer leads exist and may be used in this disclosure, such as, without limitation, 6-lead ECGs, like the AliveCor 6-lead ECG, single lead ECGs and the like. ECGs are able to assess cardiac rhythm, detection of myocardial ischemia and infarction, conduction system abnormalities, preexcitation, long QT syndromes, atrial abnormalities, ventricular hypertrophy, pericarditis, and/or other similar conditions. The signals of a patient's heart are shown as waves, which can then be read to indicate potential and current issues with the rhythm of their heart which may implicate certain medical diagnoses. As a nonlimiting example an ECG device 1108, may further include, but is not limited to 1-lead, 2-lead, and so on. A 6-lead ECG may include leads I, II, III, aVL, aVF, and aVR. Further information regarding ECG data may be found with reference to FIG. 12.

With continued reference FIG. 11, in some embodiments ECG data 1110 may be extracted from a static image. Static image may be in any image format including without limitation bitmap, joint photographic experts group (jpeg), graphics interchange format (gif), tag image file format (tiff), portable document format (PDF), or the like. Static image may be sourced from a sensor which may include any sensor capable of collecting time series data including, without limitation, a device for capturing an electrocardiogram (ECG), also known as an ECG-enabled device, including without limitation any ECG device having any number of leads and/or electrodes, including without limitation a 12-lead ECG machine such as a Biocare 12-lead ECG machine, a 6-lead ECG machine, an exercise ECG machine, a Holter monitor, a wearable device such as an exercise ECG tracker, a smart watch having a wrist sensor, or the like, and/or any other device capable of capturing ECG data and/or any component thereof. This sensor may alternatively or additionally include any type of device capable of capturing electroencephalograms (EEGs), magnetic resonance imagers, electromyography scans (EMGs), galvanic skin response sensors, fitness trackers, blood pressure monitors, sleep trackers, blood-oxygen level monitors, heart rate trackers, diabetes or herpes trackers, immune disorder logs, or any other medical imaging or time series data capable of being plotted. At least a sensor may refer to standalone devices, such as those used exclusively in established medical facilities, such as magnetic resonance imagers, computerized tomography scans, x-rays, ultrasounds, radiotherapy equipment, intravenous monitors, or any other standalone device. While this disclosure openly discusses medical devices, the disclosure applies to any time series collection devices including non-medical applications wherein the exportable information is limited to static images of the time series data. Additionally, at least a sensor may be a plurality of handheld, or wearable devices such as a Fitbit watch or wristband or other wearable heart rate monitor, a pacemaker or other cardiac rhythm management implant, glucose monitor, smart watch, real-time blood pressure sensors, temperature monitors, respiratory rate monitors or other biosensors, or any other wearable monitor.

Still referring FIG. 11, for the purposes of this disclosure, “static image time series of measured values” is a digital or printed image compiling information usually derived from a digital device interrogation output, formatted based on the source device protocols and containing time series data capable of being plotted on a two-dimensional axis. As used herein, “static” implies that all image data, metadata, and numerical information contained within the image, even when digitally stored, is inaccessible for processing outside of a human or machine interpreting the image and translating it to a different format that may be interacted with or digitally extracted. In a non-limiting embodiment, a screenshot of a chart is generally considered static data since the data cannot be digitally extracted, but rather only visually observed. Conversely, a dynamic image may include an Excel chart as viewed within Excel, or a time series readout directly within an ECG machine, or any image where the underlying data may be exported to a .csv or similar file format. Static time series image may be in image format, wherein the discrete data points may be identified and interpreted from the image. In other applications of Generative Adversarial Networks, inputs may include a plurality of different types or domains, including without limitation text, code, images, molecules, audio (e.g., music), video, and robot actions (e.g., electromechanical system actions). As a non-limiting example, an ECG recording's data set of voltage measured over a 30-second period at a frequency ranging from 50 Hz-500 Hz may be plotted, recorded, and saved or printed for use by processor 1104 as static time series image. Time-plotted voltages, especially within the range of voltages expected to be detected from a human heart through skin contact, exported from any capable device, may be used for static time series image. In a non-limiting embodiment, static time series image may further include any set of plotted time series data which may be valuable within a separate set of domain protocols other than its original static image source.

Still referring FIG. 11, static time series image, in a non-limiting embodiment may be input in a multitude of source formats including Portable document format (PDF), Portable Network Graphics (PNG), Joint Photographic Experts Group (JPEG), Tagged Image File (TIFF), Bitmap Image File (BMP), Photoshop Document (PSD), Encapsulated Postscript (EPS), Adobe Illustrator Document (AI), Adobe Indesign Document (INDD), Raw Image Format (RAW) or other static image formats. Processor 1104 may then convert all static time series image into a single format while converting the data internally, or processor 1104 may support direct conversion from each distinct input format option to any designated target domain protocol format.

Still referring FIG. 11, processor 1104 is configured to convert the static time series image into a single format by parsing the received time series of measured values into vector data representing the source data, identifying the specific sensor associated with each vector data when multiple sensors are combined into a single time series, converting the vector data to data points, and scaling and aligning the converted data points. For instance, converted data points may be scaled along a time axis (e.g., horizontal axis) and aligned on a signal axis (e.g., vertical axis). Time axis and signal axis may span two-dimensions; in some cases, time axis may be orthogonal to signal axis. Signal axis may represent signal values, e.g., lead voltage from ECG. As used herein, “parsing” refers to the process of separating and analyzing the individual time series sensor inputs while retaining the relationships between the parsed data and all affiliated data. In a non-limiting embodiment, an ECG may contain twelve or more individual leads, each transmitting a separate time series of voltages plotted over time. Each individual ECG lead time series may be separated into individual vector data sets containing the voltage values and their affiliated times as individual data points, and independently analyzed by processor 1104. Processor 1104 may additionally enable converting the time series data points to various time-segmented time series data sets. In a non-limiting embodiment, a 10 second ECG time series may be clipped down to a 2.5 second or 5 second ECG, or segmented into these or other smaller time increments as directed by the user.

Further referring FIG. 11, as a non-limiting example, static time series image may include a patient's blood pressure plotted over a specified time, heart rate, blood-sugar, stress test data, or any relevant time series data associated with a specified initial domain protocol. Static time series image may additionally contain identifying or descriptive data meant primarily to support the targeted time series data. For example, static time series image may include timing information for when the time series was initially recorded, appended notes from medical professionals, location data, or any other appropriate information. These additional data tags embedded within the static time series image may be used as training data to support pairing input data to output data. Specifically, in a non-limiting embodiment, in the example of an ECG time series, various inputs and grouping mechanisms may aid diagnosis of a cardiac irregularity, which may be an indicator of an atrial or ventricular fibrillation. Once confirmed by a medical professional, especially if in multiple instances of repeating similar circumstances, the machine learning model may identify patterns across these instances such that it could grow to act as an early warning system for more severe conditions. Continuing in this non-limiting embodiment, various types of input data included in static time series image may be grouped together in a logical manner to support these types of early warning diagnosis support. In an additional non-limiting embodiment, heart rate training data may support detecting and diagnosing a tachycardia or bradycardia condition, each of which may be indicative of severe or complex issues needing immediate response care. Additionally, blood pressure, electromyography data, computerized tomography (CT) scans, magnetic resonance imaging (MRI), or any other device where data is collected over time and is operative only within an initial domain protocol may be included in static time series image.

Still referring FIG. 11, static time series image may consist of various ECG formats. In a non-limiting embodiment, a 12-lead ECG may use various recording formats including 3×4, 3×4+R, 3×4+3R, 6×2, 6×2+R, 6×2+3R, 12, 12+R, 12+3R, and/or rhythm mode, then may store the data so recorded within its proprietary system; such a device may enable exporting the collected data to a JPEG, PNG, TIFF, Bitmap, GIF, EPS, RAW image file, or other form of digital image. Additionally, any type of image of time series data may be screenshot and/or printed and subsequently used as input for static time series image. As a further non-limiting example, an ECG machine may use application of Minnesota Code, CSE and/or AHA database formatting guidelines, as well as support for an ECG management system or HL7 protocol. Each of these specified formats and data exchange protocols may be interoperable with other ECG devices, but they may be exclusive to a hardware sensor and/or sensor component relied upon to generate the data.

Still referring FIG. 11, static time series image may include multiple time series each with separate domain protocol formats. Specifically, processor 1104 may receive static time series image that may include a first time series and a second time series; each of first time series and second time series may include time-series data pertaining to the same category of process being measured, such as time-series data from the same type of diagnostic process or the like. In an embodiment, first time series may be recorded by and/or received from a first device while second time series may be recorded by and/or received from a second device; first device and second device may be different devices and/or different types of devices and may record using the same initial domain protocol as each other or may record in two distinct initial domain protocols. Initial time series data may include a plurality of sets of time series data from a plurality of devices, of which any two devices may include a first device and second device as described above; such initial time series data may include datasets in a plurality of distinct initial domain protocols. As a non-limiting example, multiple ECG data sets, which may be recorded with multiple initial domain protocols, may be used as static time series image to develop a single common protocol for the data sets from each ECG as described in further detail below. Conversion of initial domain protocols to a common protocol may enable a medical professional to use any or all such datasets within either or both hardware configurations to analyze the ECG data. Development of a common domain protocol may be used to support future conversions.

With continued reference FIG. 11, static time series image may be received through a network of connected devices. In a non-limiting embodiment, a device that captures one or more elements of time series data and/or performs one or more steps described in this disclosure may be communicatively connected to one or more other devices, including without limitation any devices described in this disclosure, a local area network (LAN), a wide area network (WAN) such as the Internet or a subset thereof, such that all recorded data may be accessible via any other web enabled device. In this way, static time series image may be requested and imported into processor 1104 via web or local network interface. Processor and/or another device may divide processing tasks between multiple processors to accelerate delivery of completed dynamic time series data.

With continued reference FIG. 11, static time series image may be received through a direct file importing process, wherein static time series image may be saved and downloaded to processor 1104. This may include file transfers from any type of hard drive or other memory type exchange or replication. Static time series image may be locally generated in cases where processor 1104 is built in conjunction with or contained within an ECG-capable device. Static time series image may also be imported into processor 1104 through manual generation, wherein a user populates all necessary data by any mechanism wherein the minimum required set of time series data is made available to apparatus 1100.

With continued reference FIG. 11, static time series image may be received from a scanning device. In cases where the time series data is only available in a tangible, paper format, the image may be scanned in using any scanner with sufficient clarity in its scanning process. Processor 1104 may allow for direct ingestion of the scanned time series image or may support a conversion to a preferred image format. Scanning of the time series image may be accomplished in any manner capable of generating a digital representation of the time series image to include mobile phone image scans, drum scanner scans, flatbed scans, or others.

Still referring FIG. 11, processor 1104 may rely on optical character recognition or optical character reader (OCR), executed by processor 1104 to automatically convert images of written (e.g., typed, handwritten or printed) text into machine-encoded text. In some cases, recognition of at least a keyword from an image component may include one or more processes, including without limitation optical character recognition, optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring FIG. 11, in some cases OCR may be an “offline” process, which analyzes a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring FIG. 11, in some cases, OCR processes may employ pre-processing of image component. Pre-processing may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases, a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring FIG. 11, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

Still referring FIG. 11, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning processes like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIG. 5 below. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

Still referring FIG. 11, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool includes OCRopus. OCRopus development is led by German Research Centre for Artificial Intelligence in Kaiserslautern, Germany. In some cases, OCR software may employ neural networks, for example neural networks as taught in reference to FIGS. 6-7 below.

Still referring FIG. 11, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make use of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

Still referring FIG. 11, image recognition and processing may build upon the character recognition methods discussed above. Time series data is generally less complex to interpret than the infinite possible image types, so a predefined analysis which targets time series types of data may be efficiently translated to an interrogable format, such that the image data may have numerical statistics applied and an underlying algorithm to define the time series graph. As used herein, an “interrogable” format is a format in which the individual data points that make up the time series are both quantified and accessible by processor 1104 and the user for processing and analysis. For instance, and without limitation, an interrogable format may permit processor 1104 and/or a user to isolate a specific data point such as a value at a set time, a sample number, the sample rate used, or any numeric value associated with any part of the time series. As a further example, in an interrogable format, time series data be possible to be retrieved and/or analyzed using indices, similarly to a vector or array data structure. Interrogable format may alternatively or additionally permit retrieval of entries according to times, which may map to indices and/or act as a substitute therefor. Alternatively or additionally, interrogable format may be configured to permit retrieval of time and/or indices of entries having specified values and/or falling into a range of specified values. As a further alterative or additional example, interrogable format may be configured to retrieve indices, times, values, and/or ranges of values corresponding to peaks, troughs, system and/or user-entered threshold values, first, second, or higher-order derivatives of curves and/or linearized and/or localized approximations thereof, or any other mathematical or other characteristic of a curve or other graphical object represented in interrogable format. Alternatively or additionally, an interrogable format may be a format that can return a set of values belonging to a range, such as a range of values over a period of a periodic signal, a range of values over a fraction of a period such as a half-period, quarter-period, or the like, a range of values found between two consecutive specified points as described above, including between two peaks, troughs, zeros, zeros of first, second, or higher-order derivatives, or the like. As an additional example, interrogable format may permit and/or be configured to perform retrieval of samples from a portion of a graph and/or signal that matches a particular pattern, such as a pattern representing a specific cardiac event such as a “heart murmur,” Q-waves, delta waves, Brugada syndrome signal elements, QRS-end slurring and/or notching, Digoxin effects, arrythmias, and/or other elements of interest in analysis of a signal. Pattern matching may be performed, without limitation, using a classifier, which may include any classifier as described in this disclosure. Classifier may be trained using training data correlating sequences of samples with matching sequences of samples and/or labels as entered, for instance, by an expert user such as a medical provisional; in some embodiments, a query may include a sequence to be matched with sequences within a signal in interrogable format, while in other embodiments query may include a label, for instance by way of classifying different sequences within interrogable signal to labels that can then be matched to queries containing similar labels. Any or all of the data structures and/or elements used for retrieval may be a part of a data structure instantiating interrogable format and/or may be maintained and/or utilized separately on apparatus or other devices. Interrogation of time series data may further support additional analysis including medical diagnoses, outlier erred data, or other identifiable information from the quantified dataset. Translation of the raw image into a numerically defined time series format may rely and/or include optical character recognition described above to interpret axes and/or labels of data. With the axes and/or labels defined, time series numerical characteristics may be applied based on the timing and relative locations of peaks and troughs, consistency of the waveform, amplitudes, and any other identifiable features.

Still referring FIG. 11, processor 1104 is configured to convert at least a static time series image to a dynamic time series dataset within a target domain protocol. Conversion of the static time series image to a dynamic time series data may use an unsupervised generative machine-learning process. As used in this disclosure, a “target domain protocol” is a domain protocol to which data received in one or more initial domain protocols is converted; target domain protocol may be a distinct domain protocol from each initial domain protocol or may be one of a plurality of initial domain protocols. Where two or more domain protocols exist for a given category of time-series data, target domain protocol may serve as a common domain protocol into which all other domain protocols may be converted, for instance and without limitation permitting use of all such converted datasets as training data and/or inputs for a machine-learning model, display of all such converted datasets at or by a given device that can accept and/or display data using target domain protocol, or the like. As used herein, a “common domain protocol” is a selected protocol to which all the different domains are translated so they can be used together in a process such as a machine-learning process. While a common domain protocol may not be a required transition for all time series conversions, in many cases it may allow for a standardized conversion process and gain efficiency within processor 1104 operations. In some embodiments, processor 1104 may be used to generate a universal common domain such that the common domain may act as the target domain and/or be used as real example. Use of a common domain may allow for an immediate conversion of all time series data sets immediately after generation such that all data of a specific type may be collocated and compatible within the common domain. A common domain time series data may then either be converted to a separate, user-specified target domain by repetition of processes for conversion as described in this disclosure, or it may be used as it exists in the common domain format. In this way, a common domain implementation may be used as an intermediary interpretation, to enable the comparing and contrasting of multiple sources of time series data, while also simplifying the conversion from the common domain to the various target domains. Use of a common domain protocol may simplify various conversions by converting from thousands of device domains to a single, unitary domain format.

With continued reference to FIG. 11, further disclosure regarding converting a static image to ECG data 1110 may be found in U.S. Nonprovisional application Ser. No. 18/591,499 (Attorney Docket No 1518-108USU1), filed on Feb. 29, 2024, and entitled “APPARATUS AND METHOD FOR TIME SERIES DATA FORMAT CONVERSION AND ANALYSIS,” U.S. Nonprovisional application Ser. No. 18/641,217 (Attorney Docket No. 1518-123USU1), filed on Apr. 19, 2024, and entitled “SYSTEMS AND METHODS FOR TRANSFORMING ELECTROCARDIOGRAM IMAGES FOR USE IN ONE OR MORE MACHINE LEARNING MODELS,” and U.S. Nonprovisional application Ser. No. 18/652,364 (Attorney Docket No 1518-124USU1), filed May 1, 2024, and entitled “Apparatus and Method for Training a Machine Learning Model to Augment Signal Data and Image Data,” the entirety of each of which is incorporated herein by reference.

With continued reference FIG. 11, memory 1106 may include instructions configuring the at least a processor 1104 to receive ECG data 1110 from ECG device 1108. In some embodiments, processor 1104 may receive ECG data 1110 through a wired connection. A wired connection, as non-limiting examples, may include ethernet, coax, USB, TRS, HDMI, RCA, XLR, component, and the like. In some embodiments, processor 1104 may receive ECG data 1110 through a wireless connection. A wireless connection, as non-limiting examples, may include WiFi, radio, NFC, BlueTooth, Cellular data, 2G, 3G, 4G, LTE, 5G, and the like.

With continued reference FIG. 11, processor 1104 and/or computing device 1102 may be communicatively connected ECG device 1108. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference FIG. 11, memory 1106 includes instructions configuring the at least a processor 1104 to generate, using a repeat-ablation efficacy machine-learning model 1113, repeat ablation efficacy data 1115 representing predicted efficacy of a repeat ablation procedure. In some embodiments, repeat ablation procedure may be to resolve an arrhythmia of patient 1112. For the purposes of this disclosure, a “repeat ablation procedure” is an ablation procedure that is performed on a patient that has already undergone at least another ablation procedure.

With continued reference FIG. 11, in some embodiments, repeat-ablation efficacy machine-learning model 1113 may be configured to receive as input post-ablation arrhythmic

ECG data. In some embodiments, processor 1104 may be configured to input, into repeat-ablation efficacy machine-learning model 1113, post-ablation arrhythmic ECG data.

With continued reference FIG. 11, in some embodiments, repeat-ablation efficacy machine-learning model 1113 may be configured to output repeat-ablation efficacy data 1115. In some embodiments, processor 1104 may be configured to receive as output, from repeat-ablation efficacy machine-learning model 1113, repeat-ablation efficacy data 1115.

With continued reference FIG. 11, in some embodiments, repeat-ablation efficacy data 1115 may include a quantitative value. For example, quantitative value may include a probability. For example quantitative value may include a probability that a recurrent case of Afib may be addressed through a repeat ablation procedure. In some embodiments, repeat-ablation efficacy data may be a quantitative value, such as “good,” “bad,” “ok,” and the like. In some embodiments, repeat-ablation efficacy data may include a classification. In some embodiments, classification may be based on the relative likelihood of success of a repeat ablation procedure. In some embodiments, classification may include a suggested next treatment.

With continued reference FIG. 11, repeat-ablation efficacy machine-learning model 1113 may include, as non-limiting examples, treatment machine-learning model 1136 and/or ablation evaluation machine-learning model 1114.

With continued reference FIG. 11, repeat-ablation efficacy machine-learning model 1113 may be trained using ablation training data. In some embodiments, ablation training data may be retrieved from an EHR database 1120. In some embodiments, ablation training data may include historical electrocardiogram data correlated to historical ablation data. In some embodiments, historical ablation data may include historical repeat ablation efficacy data. In some embodiments, ablation training data may be de-identified consistent with other training data in this disclosure.

With continued reference FIG. 11, memory 1106 includes instructions configuring the at least a processor 1104 to generate, using an ablation evaluation machine-learning model 1114, ablation evaluation data 1118 of patient 1112. For the purposes of this disclosure, “ablation evaluation data,” is data related an evaluation of pulmonary vein reconnection. For example, ablation evaluation data 1118 may include a predicted likelihood of pulmonary vein reconnection. E.g., 10%, 20%, 50%, and the like. Ablation evaluation data may include detecting whether or not there are pulmonary vein reconnections. This may be undesirable as it may cause certain medical issues of patient 1112 to begin to reoccur, such as atrial fibrillation. Thus accurate ablation evaluation data 1118 may allow for detection of patients 1112 wherein medical issues are likely to reoccur. In some embodiments, ablation evaluation data 1118 may further include a determination as to whether a pulmonary vein has connected or (re) connected to the heart's electrical conduction circuit, wherein such reconnection is identified as a potential cause or contributing factor to recurrence of atrial fibrillation in patient 1112. Ablation evaluation machine-learning model 1114 may be consistent with any machine-learning model disclosed in this disclosure. In some embodiments, ablation evaluation machine-learning model 1114 may be created using a machine-learning module, such as machine-learning module 400 disclosed with reference to FIG. 4.

With continued reference FIG. 11, memory 1106 contains instructions configuring processor 1104 to train an ablation evaluation machine-learning model 1114 using ablation evaluation training data 1116. Ablation evaluation training data 1116 may include historical ECG data 1107 correlated to historical ablation evaluation training data 1116. In some embodiments, ablation evaluation training data 1116 may be retrieved from a database. In some embodiments, ablation evaluation training data 1116 may be retrieved from an electronic health record (EHR) database 1120.

With continued reference FIG. 11, an “electronic health record database,” for the purposes of this disclosure, is a database that contains digital information concerning one or more patients' medical history. EHR database 1120 may store one or more patient's EHR. An “electronic health record,” for the purposes of this disclosure is a digital record of a patient's health history. An EHR may include a digital system for storing patient and population health information in a standardized format, enabling easy sharing across various healthcare settings. EHRs may encompass a wide range of data, including patient demographics, medical history, medications, allergies, immunization records, lab results, radiology images, vital signs, and billing details. They may enhance healthcare quality by providing comprehensive data for care management programs, facilitating the development of new treatments, and improving healthcare delivery. EHRs may ensure accurate, up-to-date, and legible records, reduce the risk of data replication, and promote better communication between patients and providers. Additionally, EHRs may support population-based studies by enabling efficient data extraction and analysis of long-term patient trends.

With continued reference FIG. 11, databases, as described in this disclosure, may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

With continued reference FIG. 11, in some embodiments, EHR database 1120 may be located remotely from computing device 1102. For example, in some embodiments, EHR database 1120 may be located in the cloud. In some embodiments, EHR database 1120 may be located locally at computing device 1102.

With continued reference FIG. 11, “training data,” as used in this disclosure, is data containing correlations that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by processor 1104 may correlate any input data as described in this disclosure to any output data as described in this disclosure.

With continued reference FIG. 11, memory 1106 contains instructions configuring processor 1104 to generate ablation evaluation training data 1116. In some embodiments, generating ablation evaluation training data 1116 may include retrieving historical ECG data 1107 correlated to historical ablation performance data 1124. “Historical ECG data,” for the purposes of this disclosure, is ECG data that has been collected in the past and not from a current patient. “Historical ablation performance data,” for the purposes of this disclosure, is ablation performance data relating to past users and patient, not a current patient.

With continued reference FIG. 11, in some embodiments, processor 1104 may be configured to receive ablation evaluation training data 1116 from EHR database 1120. In some embodiments, historical ECG data 1107 may be received from EHR database 1120. In some embodiments, historical ablation performance data 1124 may be received from EHR database 1120.

With continued reference FIG. 11, in some embodiments, training ablation evaluation machine-learning model 1114 may include receiving a plurality of patient health records 1126 from EHR database 1120. A “patient health record,” for the purposes of this disclosure is a record of the medical history of a patient. In some embodiments, processor 1104 may be configured to identify a subset of patient health records 1128 from an EHR database 1120. In some embodiments, subset of patient health records 1128 may be a subset of patient health records 1128. Processor 1104 may be configured to identify a subset of patient health records 1128 comprising post-ablation ECG data 1130. For the purposes of this disclosure, “post-ablation ECG data” is data of one or more electrocardiograms that were performed on patients after they have undergone an ablation procedure.

With continued reference FIG. 11, identifying subset of patient health records 1128 may include using natural language processing. In some embodiments, this may include using a language processing module. Language processing module may include any hardware and/or software module. Language processing module may be configured to extract, from the one or more documents, one or more words. One or more words may include, without limitation, strings of one or more characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring FIG. 11, language processing module may operate to produce a language processing model. Language processing model may include a program automatically generated by computing device and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

Still referring FIG. 11, language processing module and/or diagnostic engine may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein are statistical models with inference algorithms that that may be applied to the models. In such models, a hidden state to be estimated may include an association between extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Alternatively or additionally, and with continued reference FIG. 11, language processing module may be produced using one or more large language models (LLMs), neural networks, and the like.

Continuing to refer FIG. 11, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring FIG. 11, language processing module may use a corpus of documents to generate associations between language elements in a language processing module, and diagnostic engine may then use such associations to analyze words extracted from one or more documents and determine that the one or more documents indicate significance of a category. In an embodiment, language module and/or processor 1104 may perform this analysis using a selected set of significant documents, such as documents identified by one or more experts as representing good information; experts may identify or enter such documents via graphical user interface, or may communicate identities of significant documents according to any other suitable method of electronic communication, or by providing such identity to other persons who may enter such identifications into processor 1104. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, diagnostic engine may automatically obtain the document using such an identifier, for instance by submitting a request to a database or compendium of documents such as JSTOR as provided by Ithaka Harbors, Inc. of New York.

With continued reference FIG. 11, processor 1104 may use language processing module to identify patient health records 1126 that include post-ablation ECG data 1130. For example, processor 1104 may use language processing module to detect data elements for each part of a patient health record 1126 and, if patient health record 1126 contains an ECG procedure that occurs temporally after an ablation procedure. An “ablation procedure,” for the purposes of this disclosure, is a medical procedure that involves removing or destroying tissue within the body. Ablation procedures may include, as non-limiting examples, radiofrequency ablation, cryoablation, maze ablation, laser ablation, heat ablation, pulsed-field ablation (PFA), and the like.

With continued reference FIG. 11, in some embodiments, processor 1104 may be configured to identify subset of patient health records 1128 containing post-ablation ECG data 1130 using a patient health record classifier. Patient health record classifier may be consistent with any classifier disclosed in this disclosure. Patient health record classifier may be trained using health record training data. Health record training data may include inputs correlated to outputs. Health record training data may include examples of patient health records 1126 labeled according to whether they contain post-ablation ECG data 1130. In some embodiments, health record training data may be labeled by medical experts.

With continued reference FIG. 11, “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Computing device 1102 and/or another device may generate a classifier using a classification algorithm, defined as a processes whereby a computing device 1102 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring FIG. 11, computing device 1102 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)=P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 1102 may then calculate a likelihood table by calculating probabilities of different data entries and classification labels. Computing device 1102 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference FIG. 11, computing device 1102 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference FIG. 11, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{Σ}_{i = 0}^{n} {{a}_{i}}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference FIG. 11, processor 1104 may be configured to further process subset of patient health records 1128 to filter out patient health records 1126 wherein the ECG occurred outside of a time window after the ablation procedure. For example, time window may be 1 week, 2 weeks, 1 month, 2 months, 3 months, 6 months, 8 months, 1 year, or 2 years. Time window may, in some embodiments, be between 1 day and 3 years. Time window may, in some embodiments, be between 1 day and 2 years. Time window may, in some embodiments, be between 1 month and 2 years.

With continued reference FIG. 11, in some embodiments, historical ablation performance data 1124 may be manually assigned for each patient health record 1126 (or, subset of patient health records 1128) by a medical professional. In some embodiments, historical ablation performance data 1124 may be identified using a language processing module as discussed above. In some embodiments, language processing module may identify keywords associated with ablation outcomes and use those keywords to identify historical ablation performance data 1124 within patient health records 1126 (or, subset of patient health records 1128).

With continued reference FIG. 11, processor 1104 may generate ablation evaluation training data 1116 from subset of patient health records 1128. In some embodiments, processor 1104 may use subset of patient health records 1128 as ablation evaluation training data 1116.

With continued reference FIG. 11, processor 1104 may be configured to train ablation evaluation machine-learning model 1114 using ablation evaluation training data. In some embodiments, this may be an iterative process, wherein ablation evaluation machine-learning model is trained and retrained until it surpasses an accuracy threshold. In some embodiments, ablation evaluation machine-learning model 1114 may be periodically retrained with updated training data. In some embodiments, ablation evaluation machine-learning model may be retrained using feedback.

With continued reference FIG. 11, in some embodiments, generating ablation evaluation training data 1116 from subset of patient health records 1128 may include de-identifying the subset of patient health records. For the purposes of this disclosure, “de-identifying the subset of patient health records” is removing personally identifying information (PII) from the patient health records. “PII,” for the purposes of this disclosure, is any information that can be used to identify, contact, or locate and individual, either directly or indirectly. Processor 1104 may be configured to identify, using language processing module, one or more instances of PII within patient health records 1126. In some embodiments, processor 1104 may be configured to replace these instances of PII with generic placeholders such as, for a name, “[NAME]” or “John,” or, for a birthday, MMDDYYY, and the like. In some embodiments, processor 1104 may be configured to, before replacing instances of PII with generic placeholders, identify false positives using a false positives list. False positives list may include a plurality of terms or tokens that, while they may resemble PII, actually are not. For example, the word Heimlich (for the Heimlich maneuver) may be incorrectly identified as PII because it is also a last name; however, Heimlich may be on the false positives list and then processor 1104 may mark it as non PII so that it does not get replaced with a generic placeholder.

With continued reference FIG. 11, memory 1106 contains instructions configuring processor 1104 to input, into ablation evaluation machine-learning model 1114, ECG data 1110. Ablation evaluation machine-learning model may be configured to receive ECG data 1110 as input and output ablation evaluation data 1118. Memory 1106 contains instructions configuring processor 1104 to receive, as output, from ablation evaluation machine-learning model 1114, ablation evaluation data 1118 of patient 1112.

With continued reference FIG. 11, in some embodiments, ablation evaluation data 1118 may include predicted chance 1132 of pulmonary vein reconnection. For the purposes of this disclosure, a “predicted chance of pulmonary vein reconnection” is a likelihood that a pulmonary vein of a patient will reconnect with the atria. In some embodiments, predicted chance 1132 of pulmonary vein reconnection may include quantitative data, such as a percent chance. In some embodiments, predicted chance 1132 of pulmonary vein reconnection may include qualitative data, such as descriptors like “likely,” “very likely,” “unlikely,” “very unlikely,” “neutral,” and the like. In some embodiments, ablation evaluation training data may include historical ECG data 1107 correlated to predicted chances 1132 of pulmonary vein reconnection.

With continued reference to FIG. 11, in one or more embodiments, processor 1104 may be configured to implement a predictive model trained on labeled patient data containing body surface ECGs and associated ground-truth reconnection statuses. In one or more embodiments, ground-truth reconnection status may be determined based on electrocardiogram data, long-term recurrence of atrial fibrillation, or other clinical outcomes. In one or more embodiments, predictive model may include a machine-learning model consistent with ablation evaluation machine-learning model 1114, repeat-ablation efficacy machine-learning model 1113 and/or consistent with any machine learning model as described in this disclosure. In one or more embodiments, ablation evaluation machine-learning model 1114 may be trained using ablation evaluation training data that includes body surface ECG data 1107 labeled with reconnection outcomes. Training may be performed using supervised learning approaches, including but not limited to, deep neural networks, support vector machines, decision trees, or ensemble methods. In one or more embodiments, ablation evaluation training data may include serial ECG recordings collected over time to capture dynamic electrophysiological changes. In one or more embodiments, processor 1104 may be further configured to identify, within ECG data 1110, one or more surface ECG features or biomarkers indicative of pulmonary vein reconnection. In one or more embodiments, ECG features or biomarkers may be stored and/or received as ablation evaluation data 1118. In one or more embodiments, features and/or biomarkers may include morphological characteristics, frequency domain metrics, signal entropy, or time-domain variations of ECG waveform components such as P-waves, atrial activity signals, or RR interval irregularity.

With continued reference to FIG. 11, processor 1104 may be configured to implement a decision-support tool that uses ablation evaluation data 1118 to categorize patient 1112 into one or more clinical decision classes. For example and without limitation, ablation evaluation data 1118 may be used to classify patient 1112 into a category indicating a likelihood of pulmonary vein reconnection. In one or more embodiments, ablation evaluation data 1118 may be used in various clinical use cases, including but not limited to post-ablation monitoring, wherein follow-up body surface ECGs are analyzed to assess PV reconnection without requiring repeat invasive diagnostics, in reablation triage, wherein ablation evaluation data 1118 guides decision-making regarding the necessity of a repeat ablation procedure, and/or in clinical trial stratification, wherein patients are grouped based on predicted PV reconnection likelihood to assess efficacy of AFib therapies or ablation strategies. In one or more embodiments, a predictive model as described herein may operate within a broader framework of input-output models for AFib ablation outcome prediction. This framework may include hierarchical modeling levels, wherein the highest-level model predicts long-term AFib recurrence, the mid-level model predicts whether PV isolation has been achieved or persists, and the lowest-level model evaluates the success of individual ablation lesions.

With continued reference to FIG. 11, in one or more embodiments, inputs to ablation evaluation machine-learning model 1114 and/or repeat-ablation efficacy machine learning model may include but are not limited to post-ablation body surface ECG data 1110, historical

ECG data 1107, electrocardiogram record, and/or other procedural metadata such as energy delivery parameters, lesion duration, or catheter positioning. Outputs from the model may include binary classification values (e.g., “PV reconnection likely”/“PV reconnection unlikely”), probabilistic estimates (e.g., 72% likelihood of reconnection), associated clinical recommendations (e.g., “Reablation advised”/“Reablation not advised”) and/or the like. In one or more embodiments, the predictive model may be further configured to generate qualitative outputs in addition to quantitative predictions. For example, predicted chance 1132 of pulmonary vein reconnection may be expressed using descriptive terms such as “likely,” “very likely,” “neutral,” “unlikely,” or “very unlikely,” to support clinician interpretation and downstream treatment planning. In one or more embodiments, ablation evaluation machine learning model 1114 and/or repeat-ablation efficacy machine-learning model 1113 may be trained with historical data containing a plurality of ECGs correlated to a plurality of clinical outcomes and/or ablation performance data. In one or more embodiments, ablation evaluation machine learning model 1114 and/or repeat-ablation efficacy machine-learning model 1113 may be trained to identify features and/or biomarkers within electrocardiograms and correlate them to clinical outcomes (i.e. ablation evaluation data). In one or more embodiments, ablation evaluation machine learning model 1114 and/or repeat-ablation efficacy machine-learning model 1113 may be configured to identify features and/or biomarkers within surface ECG data 1110 and generate ablation performance data as a result. In one or more embodiments, ablation evaluation machine learning model 1114 and/or repeat-ablation efficacy machine-learning model 1113 may be configured to identify patterns, features and/or biomarkers within historical data that is representative of a clinical outcome and use said patterns, features and/or biomarkers in order to correlate surface ECG data with clinical outcomes such as ablation evaluation data 1118. In one or more embodiments, the machine-learning models as described above may use historical data to identify features, patterns and/or biomarkers within historical ECG data that are historically representative of pulmonary vein reconnection determinations. For example, and without limitation, ablation evaluation machine learning model 1114 and/or repeat-ablation efficacy machine-learning model 1113 may be trained to identify features within historical ECG data that are indicative of pulmonary vein reconnection and/or indicative of a lack of pulmonary vein reconnection. In one or more embodiments, ablation evaluation machine learning model 1114 and/or repeat-ablation efficacy machine-learning model 1113 may be trained to identify features within historical ECG data that have been historically correlated to pulmonary vein reconnections and/or a lack thereof.

With continued reference to FIG. 11, in one or more embodiments, ablation evaluation machine-learning model 1114 and corresponding ablation evaluation data 1118 may be configured to support one or more downstream clinical decisions related to the management of atrial fibrillation post-ablation. For example, and without limitation, processor 1104 may be configured to use ablation evaluation data 1118 to determine whether pulmonary vein reconnection has likely occurred following an ablation procedure. This determination may be used to assess whether a second, or reablation, procedure is clinically warranted. In one or more embodiments, processor 1104 may compare predicted chance 1132 of pulmonary vein reconnection against one or more predefined thresholds to guide reablation recommendations. For example, if predicted chance 1132 exceeds a clinically significant threshold (e.g., 70% likelihood of reconnection), ablation evaluation data 1118 may indicate that reablation is advised. Conversely, if predicted chance 1132 falls below a lower threshold (e.g., 30%), the system may indicate that reablation is not presently necessary, thereby helping to prevent unnecessary procedures. In one or more embodiments, processor 1104 may be configured to stratify patients into subgroups based on their predicted reconnection likelihood. This stratification may allow for differential treatment planning, including identifying patients with a high likelihood of reconnection as priority candidates for early follow-up or reintervention, while patients with low likelihoods may be monitored conservatively. Such patient stratification may further support procedural planning, risk-benefit analysis, and shared decision-making between clinicians and patients. In one or more embodiments, use of ablation evaluation machine-learning model 1114 may improve overall clinical outcomes by enabling earlier detection of patients at risk for recurrent atrial fibrillation due to PV reconnection. By directing reablation efforts toward patients most likely to benefit and avoiding unnecessary procedures in those unlikely to have reconnected PVs, the system may reduce procedural burden, limit patient exposure to procedural risks (e.g., cardiac perforation, stroke, infection), and/or lower healthcare costs. In one or more embodiments, memory 1106 may include instructions that allows for ablation evaluation data 1118 to be integrated into a clinical decision-support system that automatically flags high-risk patients for electrophysiologist review or triggers predefined clinical pathways. For example, if ablation evaluation data 1118 includes a high predicted chance 1132 of reconnection, the system or apparatus 1100 may generate an alert recommending repeat imaging, additional diagnostic workup, or scheduling of a reablation consultation.

With continued reference to FIG. 11, in an embodiment, ablation evaluation machine learning model 1114 and/or repeat-ablation efficacy machine-learning model 1113 may comprise a deep neural network (DNN). As used in this disclosure, a “deep neural network” is defined as a neural network with two or more hidden layers. Neural network is described in further detail below with reference to FIGS. 5-7. In a non-limiting example, ablation evaluation machine learning model 1114 and/or repeat-ablation efficacy machine-learning model 1113 may include a convolutional neural network (CNN). For example, and without limitation, generating/determining/identifying x using ML process may include training CNN using particular training data for the ML model. and limitation e.g., generating/determining/identifying x as a function of A and/or B using trained CNN. A “convolutional neural network,” for the purpose of this disclosure, is a neural network in which at least one hidden layer is a convolutional layer that convolves inputs to that layer with a subset of inputs known as a “kernel,” along with one or more additional layers such as pooling layers, fully connected layers, and the like. In some cases, CNN may include, without limitation, a deep neural network (DNN) extension. Mathematical (or convolution) operations performed in the convolutional layer may include convolution of two or more functions, where the kernel may be applied to input data e.g., input data for ablation evaluation machine learning model 1114 and/or repeat-ablation efficacy machine-learning model 1113 through a sliding window approach. In some cases, convolution operations may enable processor 104 to detect local/global patterns, edges, textures, and any other features described herein within input data of ablation evaluation machine learning model 1114 and/or repeat-ablation efficacy machine-learning model 1113. Spatial features 140 may be passed through one or more activation functions, such as without limitation, Rectified Linear Unit (ReLU), to introduce non-linearities into the processing step of ablation evaluation machine learning model 1114 and/or repeat-ablation efficacy machine-learning model 1113. Additionally, or alternatively, CNN may also include one or more pooling layers, wherein each pooling layer is configured to reduce the dimensionality of input data while preserving essential features within the input data. In a non-limiting example, CNN may include one or more pooling layer configured to reduce the spatial dimensions of spatial feature maps by applying downsampling, such as max-pooling or average pooling, to small, non-overlapping regions of one or more features.

Still referring to FIG. 11, CNN may further include one or more fully connected layers configured to combine features extracted by the convolutional and pooling layers as described above. In some cases, one or more fully connected layers may allow for higher-level pattern recognition. In a non-limiting example, one or more fully connected layers may connect every neuron (i.e., node) in its input to every neuron in its output, functioning as a traditional feedforward neural network layer. In some cases, one or more fully connected layers may be used at the end of CNN to perform high-level reasoning and produce the final output such as, without limitation, ablation efficacy and/or any other outputs as described above. Further, each fully connected layer may be followed by one or more dropout layers configured to prevent overfitting, and one or more normalization layers to stabilize the learning process described herein.

With continued reference to FIG. 11, in an embodiment, training the ablation evaluation machine learning model 1114 and/or repeat-ablation efficacy machine-learning model 1113 (i.e., CNN) may include selecting a suitable loss function to guide the training process. In a non-limiting example, a loss function that measures the difference between the predicted output of ablation evaluation machine learning model 1114 and/or repeat-ablation efficacy machine-learning model 1113 and the ground truth 3D structure e.g., outputs in a test set may be used, such as, without limitation, mean squared error (MSE) or a custom loss function may be designed for one or more embodiments described herein. Additionally, or alternatively, optimization algorithms, such as stochastic gradient descent (SGD), may then be used to adjust the ablation evaluation machine learning model 1114 and/or repeat-ablation efficacy machine-learning model 1113 parameters to minimize such loss. In a further non-limiting embodiment, instead of directly predicting [output of the ML model], ablation evaluation machine learning model 1114 and/or repeat-ablation efficacy machine-learning model 1113 may be trained as a regression model to predict other form of the output of the ML model such as a numeric values. Additionally, CNN may be extended with additional deep learning techniques, such as recurrent neural networks (RNNs) or attention mechanism, to capture additional features and/or data relationships within data input. These extensions may further enhance the accuracy and robustness of the ablation efficacy determinations.

With continued reference FIG. 11, in some embodiments, memory 1106 may contain instructions configuring processor 1104 to determine a treatment recommendation 1134. A “treatment recommendation,” for the purposes of this disclosure, is a suggestion of an optimal treatment for a patient.

With continued reference FIG. 11, in some embodiments, memory 1106 may contain instructions configuring processor 1104 to generate, using a treatment machine-learning model 1136, treatment recommendation 1134 as a function of ECG data 1110. In some embodiments, treatment machine-learning model 1136 may be configured to receive ECG data 1110 as input. In some embodiments, treatment machine-learning model 1136 may be configured to output treatment recommendation 1134. In some embodiments, treatment machine-learning model 1136 may be configured to receive both ECG data 1110 and ablation evaluation data 1118 as input. Treatment machine-learning model 1136 may be consistent with any machine-learning model disclosed in this disclosure. In some embodiments, treatment machine-learning model 1136 may be created using a machine-learning module, such as machine-learning module 400 disclosed with reference to FIG. 4.

With continued reference FIG. 11, memory 1106 may contain instructions further configuring processor 1104 to receive treatment training data 1138. In some embodiments, receiving treatment training data 1138 may include receiving treatment training data 1138 from a database. In some embodiments, receiving treatment training data 1138 may include receiving treatment training data 1138 from EHR database 1120. In some embodiments, treatment training data may include historical ECG data 1107 correlated to historical treatment data 1140. “Historical treatment data,” for the purposes of this disclosure are historical records of treatments assigned to patients. In some embodiments, memory 1106 may contain instructions configuring processor 1104 to train treatment machine-learning model 1136 using the treatment training data 1138. In some embodiments, memory 1106 may contain instructions configuring processor 1104 to generate treatment recommendation 1134 using trained treatment machine-learning model 1136.

With continued reference FIG. 11, in some embodiments, treatment recommendation 1134 may include a secondary ablation target 1142. For the purposes of this disclosure, a “secondary ablation target” is an ablation target for a second ablation procedure, wherein the second ablation procedure is additional to the ablation procedure that a patient has already undergone. A “primary ablation target,” for the purposes of this disclosure, is the ablation target for the ablation procedure that the patient has already undergone. Primary ablation target may include pulmonary vein. In some embodiments, secondary ablation target 1142 may be the same as primary ablation target. In some embodiments, secondary ablation target 1142 may be pulmonary vein. In some embodiments, secondary ablation target 1142 may be different from primary ablation target. As non-limiting examples, secondary ablation target 1142 may include superior vena cava or posterior wall. In some embodiments, treatment training data 1138 may include historical ECG data 1107 correlated to historical secondary ablation targets 1142.

With continued reference FIG. 11, memory 1106 may contain instructions configuring processor 1104 to receive post-recommended treatment ECG 1144. A “post-recommended treatment ECG,” for the purposes of this disclosure, is ECG data from an ECG on the patient that was conducted after the treatment recommendation was carried out. In some embodiments, processor may retrieve post-recommended treatment ECG 1144 from EHR database 1120.

With continued reference FIG. 11, memory 1106 may contain instructions configuring processor 1104 to receive recommended treatment outcome data 1146. For the purposes of this disclosure, “recommended treatment outcome data” is data concerning the outcome of the recommended treatment. In some embodiments, processor may retrieve recommended treatment outcome data 1146 from EHR database 1120. In some embodiments, recommended treatment outcome data 1146 may include qualitative data concerning the relative success of the procedure. In some embodiments, recommended treatment outcome data 1146 may include quantitative data on the relative success of the procedure. In some embodiments, recommended treatment outcome data 1146 may include binary data on the success or failure of the procedure. For example, a “1” may indicate success while a “0” indicates failure.

With continued reference FIG. 11, memory 1106 may contain instructions configuring processor 1104 to retrain treatment machine-learning model 1136 as a function of post-recommended treatment ECG 1144 and recommended treatment outcome data 1146. For example, post-recommended treatment ECG 1144 and recommended treatment outcome data 1146 may be correlated together and used as a training pair when treatment machine-learning model 1136 is retrained.

With continued reference FIG. 11, memory 1106 may contain instructions configuring processor 1104 to receive ablation data 1148 from ablation device 1150. An “ablation device,” for the purposes of this disclosure, is a device that is configured to perform an ablation procedure. In some embodiments, ablation device may include a catheter. In some embodiments, ablation device may include a radiofrequency ablation device. In some embodiments, ablation device may include a cryoablation device. In some embodiments, ablation device may include a PFA ablation device.

With continued reference FIG. 11, “ablation data,” for the purposes of this disclosure, is data concerning an ablation procedure. In some embodiments, ablation data 1148 may include parameter data for ablation device. In some embodiments, ablation data 1148 may include data concerning the pulse strength and/or duration for the ablation procedure. In some embodiments, ablation data 1148 may include an ablation durability.

With continued reference to FIG. 11, further discussion of ablation data and ablation durability may be found in U.S. Non-provisional application Ser. No. 18/671,644 (Attorney Docket No. 1518-141USU1), filed on May 22, 2024, and entitled “SYSTEMS AND METHODS FOR DETERMINING DOSAGE PARAMETERS TO ENSURE DURABILITY IN TREATMENT PROCESSES,” and U.S. Non-provisional application Ser. No. 18/646,991 (Attorney Docket No. 1518-142USU1), filed on Apr. 26, 2024, and entitled “METHOD AND APPARATUS FOR PREDICTING PULSED FIELD ABLATION DURABILITY,” the entirety of each of which is incorporated herein by reference.

With continued reference FIG. 11, in some embodiments, ablation evaluation machine-learning model 1114 may be configured to receive ablation data 1148 and ECG data 1110 as input and output ablation evaluation data 1118. In some embodiments, ablation evaluation training data 1116 may include historical ECG data 1107 and historical ablation data correlated to historical ablation performance data 1124. In some embodiments, historical ablation data may be retrieved from EHR database 1120.

With continued reference FIG. 11, apparatus 1100 may further include a display device 1152. Display device 1152 may be configured to display a user interface 1154 which may be configured to receive one or more inputs from a user. In some embodiments, processor 1104 may be configured to generate user interface 1154. In some embodiments, processor 1104 may be configured to display ablation evaluation data 1118 using display device. In some embodiments, processor 1104 may be configured to display treatment recommendation 1134 using display device 1152. A “display device,” for the purposes of this disclosure, is an electronic device configured to display visual data to a user. Display device 1152 may include various screens, such as, as non-limiting examples, OLED, LCD, LED, CRT, QLED, plasma, and the like. Display device 1152 may include a monitor, television, projector, and the like.

With continued reference FIG. 11, in some embodiments, processor 1104 may be configured to transmit, for display, repeat-ablation efficacy data 1115. In some embodiments, processor 1104 may be configured to display repeat-ablation efficacy data 1115.

With continued reference FIG. 11, in some embodiments, apparatus 1100 may include a catheter 1156. A “catheter,” for the purposes of this disclosure, is a flexible tube that is configured to be inserted into the body. In some embodiments, catheter 1156 may include a mapping catheter. A “mapping catheter,” for the purposes of this disclosure, is a catheter that is configured to map human anatomy. Mapping catheter may include electrophysiology (EP) mapping catheter. In some embodiments, catheter 1156 may include an ablation catheter. In some embodiments, ablation device 1150 may include an ablation catheter. An “ablation catheter,” for the purposes of this disclosure, is a catheter that is configured to ablate tissue within the body. In some embodiments, ablation catheter may include a radio-frequency ablation catheter. In some embodiments, ablation catheter may include a cryoablation catheter. In some embodiments, ablation catheter may include a pulsed field ablation (PFA) catheter. In some embodiments, ablation catheter may include an electrode configured to apply electric pulses to tissue. In some embodiments, electrode may be configured to apply various ablation parameters, such as pulse width, pulse frequency, pulse mode, voltage amplitude, number of pulses, interphase, and/or interpulse delay. In some embodiments, ablation data 1148 may include ablation parameters as discussed above.

With continued reference FIG. 11, in some embodiments, apparatus 1100 may receive intracardiac echocardiogram (ICE) data 1158. For the purposes of this disclosure, ICE data 1158 is data collected from an ultrasound probe that is inserted into the heart of a patient. In some embodiments, ICE data 1158 may be collected using a catheter 1156, such as in ICE catheter.

With continued reference FIG. 11, in some embodiments, processor 1104 may be configured to receive ICE data 1158, ablation data 1148, ECG data, catheter data, ultrasound data, and/or CT data. In some embodiments, repeat-ablation efficacy machine-learning model 1113 may be configured to receive ICE data 1158, ablation data 1148, catheter data, ultrasound data, and/or CT data as input. In some embodiments, repeat-ablation efficacy machine-learning model 1113 may include models configured to process each mode of input data and output ablation efficacy data 1115. In some embodiments, repeat-ablation efficacy machine-learning model 1113 may include one or more multimodal models, wherein a multimodal model may be configured to receive as input multiple modes of data at once. In some embodiments, multimodal model may include a multimodal transformer.

With continued reference FIG. 11, ablation training data may include historical ECG data 1107 correlated to historical ablation data. In some embodiments, ablation training data may include historical ICE data, historical ECG data 1105, historical ablation data, historical catheter data, historical ultrasound data, and/or historical CT data correlated to historical ablation data. In some embodiments, treatment training data 1138 may include historical ICE data, historical ECG data 1105, historical ablation data, historical catheter data, historical ultrasound data, and/or historical CT data correlated to historical treatment data 1140. In some embodiments, ablation evaluation training data 1116 may include historical ICE data, historical ECG data 1105, historical ablation data, historical catheter data, historical ultrasound data, and/or historical CT data correlated to historical ablation performance data 1124. In some embodiments, historical ICE data, historical ECG data 1105, historical ablation data, historical catheter data, historical ultrasound data, and/or historical CT data may include historical prior-procedure data, wherein historical prior-procedure data is data collected during or temporally adjacent to a prior ablation procedure of a historical patient.

With continued reference FIG. 11, ICE data 1158 may include prior-procedure ICE data. “Prior-procedure ICE data,” for the purposes of this disclosure, is data collected from an ICE of a patient, wherein the collected during or temporally adjacent to a prior ablation procedure of the patient. In some embodiments, processor 1104 may be configured to receive prior-procedure ICE data. In some embodiments, processor 1104 may be configured to generate repeat-ablation efficacy data 1115 by inputting prior-procedure ICE data into repeat-ablation efficacy machine-learning model 1113.

With continued reference FIG. 11, in some embodiments, ablation data 1148 may include prior-procedure ablation data. “Prior-procedure ablation data,” for the purposes of this disclosure, is data collected from an ablation of a patient, wherein the collected during or temporally adjacent to a prior ablation procedure of the patient. In some embodiments, processor 1104 may be configured to receive prior-procedure ablation data. In some embodiments, processor 1104 may be configured to generate repeat-ablation efficacy data 1115 by inputting prior-procedure ablation data into repeat-ablation efficacy machine-learning model 1113.

Referring now to FIG. 12, an exemplary embodiment of ECG 1200 is illustrated. ECG may include a plurality of features such as P-wave, Q-wave, R-wave, S-wave, QRS complex, and T wave, as well as a plurality of parameters such a PR interval 1204, QT interval 1208, ST interval 1212, TP interval 1216, RR interval 1220, and the like. P-wave may reflect atrial depolarization (activation). For the purposes of this disclosure, a “PR interval” is the distance between the onset of P-wave to the onset of QRS complex. PR interval 1204 may be assessed to determine whether impulse conduction from the atria to the ventricles is normal. PR interval 1204 may be measured in seconds. For the purposes of this disclosure, a “QT interval” is a reflection of the total duration of ventricular depolarization and repolarization and is measured from the onset of QRS complex to the end of T-wave. The QT duration may be inversely related to heart rate; i.e., QT interval 1208 may increase at slower heart rates and decrease at higher heart rates. Therefore, to determine whether QT interval 1208 is within normal limits, it may be necessary to adjust for the heart rate. A heart rate-adjusted QT interval 1208 is referred to as a corrected QT interval 1208 (QTc interval). A long QTc interval may indicate an increased risk of ventricular arrhythmias. The QTc interval may be in the range of 0.36 to 0.44 seconds. For the purposes of this disclosure, an “RR interval” is the time between two consecutive R waves. For the purposes of this disclosure, a “QRS complex” is a representation of the depolarization (activation) of ventricles depicted between Q-, R- and S-waves, although it may not always display all three waves. Since the electrical vector generated by the left ventricle is usually many times larger than the vector generated by the right ventricle, QRS complex is a reflection of left ventricular depolarization.

With continued reference to FIG. 12, for the purposes of this disclosure, an “ST interval” is the segment of ECG that starts at the end of QRS complex and extends to the beginning of T wave; it represents the early part of ventricular repolarization. ST segment may be relatively isoelectric, meaning it is at the baseline, with minimal elevation or depression. The normal duration of ST interval 1212 is usually around 0.12 seconds. For the purposes of this disclosure, a “TP interval” is the segment of ECG that extends from the end of T wave to the beginning of the next P wave; it represents the time when the ventricles are fully repolarized and are in a resting state. The duration of TP interval 1216 may vary but is typically short, as it may represent the brief pause between cardiac cycles. Significant deviations may be associated with certain conditions affecting repolarization. For the purposes of this disclosure, an “RR interval” is the time between two consecutive R waves of ECG; it may represent the duration of one cardiac cycle, encompassing both atrial and ventricular depolarization and repolarization. RR interval 1220 may be measured in seconds and can be used to calculate heart rate (beats per minute) using

$$heart rate = \frac{60}{RR Interval}$$

(in seconds). The intervals described above may be used to determine a ventricular rate, i.e., the number of ventricular contractions (heartbeats) that occur in one minute, which may be closely related to RR interval 1220 of ECG, as the RR interval 1220 represents the time between two consecutive ventricular contractions.

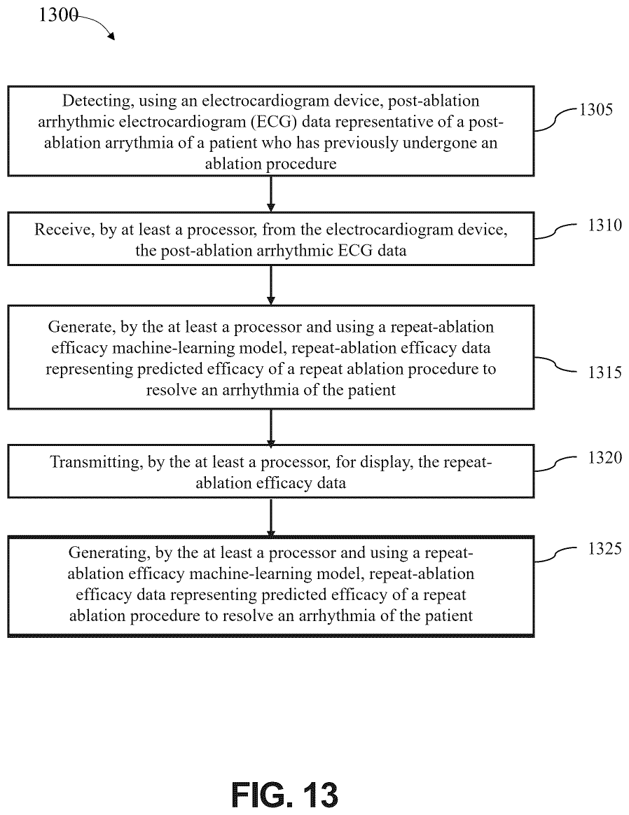

Referring now to FIG. 13, a method 1300 for prediction of pulmonary vein reconnection is illustrated. Method 1300 includes a step 1305 of detecting, using an electrocardiogram device, post-ablation arrhythmic electrocardiogram (ECG) data representative of a post-ablation arrythmia of a patient who has previously undergone an ablation procedure. In some embodiments, electrocardiogram device may include a 12-lead electrocardiogram device. This may be implemented as disclosed with reference to FIGS. 1-12, without limitation.

With continued reference to FIG. 13, method 1300 may include a step of generating, by at least a processor, training data, wherein generating the training data comprises retrieving historical electrocardiogram data correlated to historical ablation data. In some embodiments, step 1310 may include receiving a plurality of patient health records from an electronic health record database, identifying a subset of patient health records comprising a post-ablation ECG data, and generating the training data from the subset of patient health records. In some embodiments, step 1310 may include de-identifying the subset of patient health records. This may be implemented as disclosed with reference to FIGS. 1-12, without limitation.

With continued reference to FIG. 13, method 1300 may include a step of training, by the at least a processor, a repeat-ablation efficacy machine-learning model using the training data. This may be implemented as disclosed with reference to FIGS. 1-12, without limitation.

With continued reference to FIG. 13, method 1300 includes a step 1310 of receiving, by at least a processor, from the electrocardiogram device, the post-ablation arrhythmic ECG data. This may be implemented as disclosed with reference to FIGS. 1-12, without limitation.

With continued reference to FIG. 13, method 1300 includes a step 1315 of generating, by the at least a processor and using a repeat-ablation efficacy machine-learning model, repeat-ablation efficacy data representing predicted efficacy of a repeat ablation procedure to resolve an arrhythmia of the patient. Step 1325 also includes inputting, into the repeat-ablation efficacy machine-learning model, the post-ablation arrhythmic ECG data. Step 1325 also includes receiving as output, from the repeat-ablation efficacy machine-learning model, the repeat-ablation efficacy data. In some embodiments, ablation evaluation data may include a predicted chance of pulmonary vein reconnection. This may be implemented as disclosed with reference to FIGS. 1-12, without limitation.

With continued reference to FIG. 13, method 1300 includes a step of 1320 of transmitting, by the at least a processor, for display, the repeat-ablation efficacy data. This may be implemented as disclosed with reference to FIGS. 1-12, without limitation.

With continued reference to FIG. 13, method 1300 may include a step of determining, by the at least a processor, a treatment recommendation, wherein determining the treatment recommendation comprises generating, using a treatment machine-learning model, a treatment recommendation as a function of the electrocardiogram data. This step may further include receiving treatment training data, wherein the treatment training data comprises historical electrocardiogram data corelated to historical treatment data, training the treatment machine-learning model using the treatment training data, and generating the treatment recommendation using the trained treatment machine-learning model. In some embodiments, method 1300 may further include a step of displaying, using a display device, the treatment recommendation. In some embodiments, method 1300 may further include a step of receiving, by the at least a processor, a post-recommended treatment electrocardiogram and recommended treatment outcome data. This may be implemented as disclosed with reference to FIGS. 1-12, without limitation. In some embodiments, method 1300 may further include a step of retraining, by the at least a processor, the treatment machine-learning model as a function of the post-recommended treatment electrocardiogram and the recommended treatment outcome data. This may be implemented as disclosed with reference to FIGS. 1-12, without limitation. In some embodiments, the treatment recommendation may include a secondary ablation target. This may be implemented as disclosed with reference to FIGS. 1-12, without limitation.

With continued reference to FIG. 13, in one or more embodiments, method 1300 includes the steps of receiving, by at least a processor and from an electrocardiogram device, post-ablation arrhythmic electrocardiogram (ECG) data, wherein the electrocardiogram device is configured to detect the post-ablation arrhythmic ECG data representative of a post-ablation arrythmia of a patient who has previously undergone an ablation procedure, predicting, by at the least a processor and using a repeat-ablation efficacy machine-learning model, a determination of a pulmonary vein reconnection by identifying features within the post-ablation arrhythmic ECG data that are historically representative of pulmonary vein reconnection determinations and transmitting, by the at least a processor and for display, the determination. In one or more embodiments, the determination of the pulmonary vein reconnection includes a predicted chance of pulmonary vein reconnection. In one or more embodiments, predicting the determination of the pulmonary vein reconnection further includes comparing predicted against one or more predefined thresholds. In one or more embodiments, the determination includes a probability that a recurrent case of atrial fibrillation can be addressed through a repeat ablation procedure. In one or more embodiments, method 1300 further includes generating, by the at least a processor, a treatment recommendation based on the determination and transmitting, for display, the determination further includes transmitting, for display the treatment recommendation. In one or more embodiments, the treatment recommendation includes a secondary ablation procedure. In one or more embodiments, the determination includes a predicted efficacy of a repeat ablation procedure. In one or more embodiments, the repeat-ablation efficacy machine-learning model includes a multimodal model configured to receive multiple modes of data at once. In one or more embodiments, at least a first mode of data of the multiple modes of data includes ablation data received from an ablation device and at least a second mode of data of the multiple modes of data includes the post-ablation arrhythmic ECG data. In one or more embodiments, predicting the determination of the pulmonary vein reconnection further includes stratifying the patient into a subgroup for differential treatment planning. This may be implemented with reference to FIGS. 1-12 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

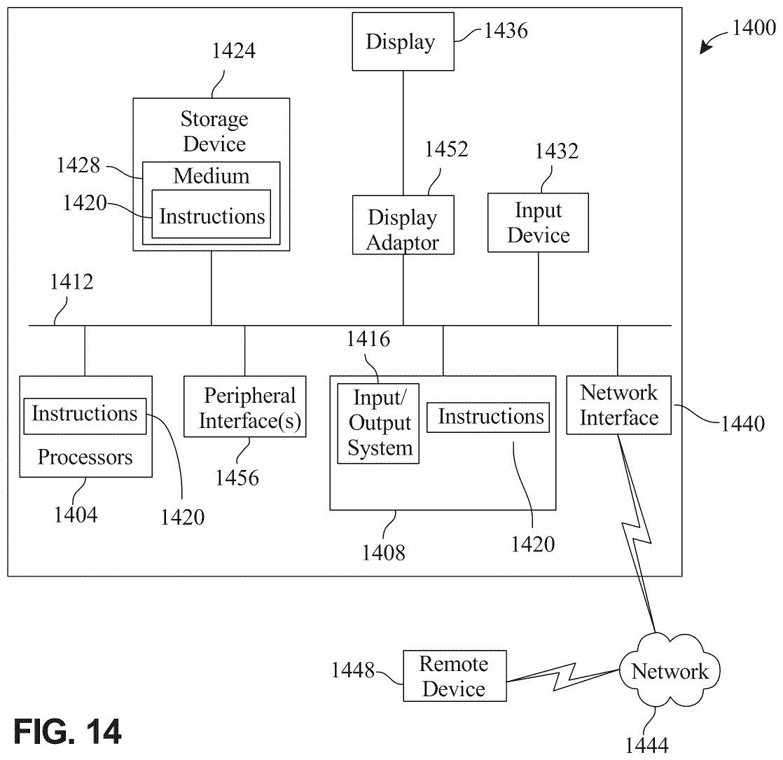

FIG. 14 shows a diagrammatic representation of one embodiment of a computing device in the exemplary form of a computer system 1400 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1400 includes a processor 1404 and a memory 1408 that communicate with each other, and with other components, via a bus 1412. Bus 1412 may include any of several types of bus structures including, but not limited to, a memory bus, a memory controller, a peripheral bus, a local bus, and any combinations thereof, using any of a variety of bus architectures.

Processor 1404 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1404 may be organized according to Von Neumann and/or Harvard architecture as a non-limiting example. Processor 1404 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1408 may include various components (e.g., machine-readable media) including, but not limited to, a random-access memory component, a read only component, and any combinations thereof. In one example, a basic input/output system 1416 (BIOS), including basic routines that help to transfer information between elements within computer system 1400, such as during start-up, may be stored in memory 1408. Memory 1408 may also include (e.g., stored on one or more machine-readable media) instructions (e.g., software) 1420 embodying any one or more of the aspects and/or methodologies of the present disclosure. In another example, memory 1408 may further include any number of program modules including, but not limited to, an operating system, one or more application programs, other program modules, program data, and any combinations thereof.

Computer system 1400 may also include a storage device 1424. Examples of a storage device (e.g., storage device 1424) include, but are not limited to, a hard disk drive, a magnetic disk drive, an optical disc drive in combination with an optical medium, a solid-state memory device, and any combinations thereof. Storage device 1424 may be connected to bus 1412 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1424 (or one or more components thereof) may be removably interfaced with computer system 1400 (e.g., via an external port connector (not shown)). Particularly, storage device 1424 and an associated machine-readable medium 1428 may provide nonvolatile and/or volatile storage of machine-readable instructions, data structures, program modules, and/or other data for computer system 1400. In one example, software 1420 may reside, completely or partially, within machine-readable medium 1428. In another example, software 1420 may reside, completely or partially, within processor 1404.

Computer system 1400 may also include an input device 1432. In one example, a user of computer system 1400 may enter commands and/or other information into computer system 1400 via input device 1432. Examples of an input device 1432 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1432 may be interfaced to bus 1412 via any of a variety of interfaces (not shown) including, but not limited to, a serial interface, a parallel interface, a game port, a USB interface, a FIREWIRE interface, a direct interface to bus 1412, and any combinations thereof. Input device 1432 may include a touch screen interface that may be a part of or separate from display device 1436, discussed further below. Input device 1432 may be utilized as a user selection device for selecting one or more graphical representations in a graphical interface as described above.

A user may also input commands and/or other information to computer system 1400 via storage device 1424 (e.g., a removable disk drive, a flash drive, etc.) and/or network interface device 1440. A network interface device, such as network interface device 1440, may be utilized for connecting computer system 1400 to one or more of a variety of networks, such as network 1444, and one or more remote devices 1448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1444, may employ a wired and/or a wireless mode of communication. In general, any network topology may be used. Information (e.g., data, software 1420, etc.) may be communicated to and/or from computer system 1400 via network interface device 1440.

Computer system 1400 may further include a video display adapter 1452 for communicating a displayable image to a display device, such as display device 1436. Examples of a display device include, but are not limited to, a liquid crystal display (LCD), a cathode ray tube (CRT), a plasma display, a light emitting diode (LED) display, and any combinations thereof. Display adapter 1452 and display device 1436 may be utilized in combination with processor 1404 to provide graphical representations of aspects of the present disclosure. In addition to a display device, computer system 1400 may include one or more other peripheral output devices including, but not limited to, an audio speaker, a printer, and any combinations thereof. Such peripheral output devices may be connected to bus 1412 via a peripheral interface 1456. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for prediction of repeat ablation efficacy, the apparatus comprising:
an electrocardiogram device, wherein the electrocardiogram device is configured to detect post-ablation arrhythmic electrocardiogram (ECG) data representative of a post-ablation arrythmia of a patient who has previously undergone an ablation procedure;
at least a processor; and
a memory communicatively connected to the at least a processor, wherein the memory contains instructions configuring the at least a processor to:
receive, from the electrocardiogram device, the post-ablation arrhythmic ECG data;
predict, using a repeat-ablation efficacy machine-learning model, a determination of a pulmonary vein reconnection comprising identifying features within the post-ablation arrhythmic ECG data that are historically representative of pulmonary vein reconnection determinations; and
transmit, for display, the determination.

2. The apparatus of claim 1, wherein the determination of the pulmonary vein reconnection comprises a predicted chance of pulmonary vein reconnection.

3. The apparatus of claim 2, wherein predicting the determination of the pulmonary vein reconnection further comprises comparing the predicted chance against one or more predefined thresholds.

4. The apparatus of claim 1, wherein the determination comprises a probability that a recurrent case of atrial fibrillation can be addressed through a repeat ablation procedure.

5. The apparatus of claim 1, wherein the at least a processor is further configured to:
generate a treatment recommendation based on the determination; and
transmit for display, the treatment recommendation.

6. The apparatus of claim 5, wherein the treatment recommendation comprises a secondary ablation procedure.

7. The apparatus of claim 1, wherein the determination comprises a predicted efficacy of a repeat ablation procedure.

8. The apparatus of claim 1, wherein the repeat-ablation efficacy machine-learning model comprises a multimodal model configured to receive multiple modes of data as input.

9. The apparatus of claim 8, wherein at least a first mode of data of the multiple modes of data comprises ablation data received from an ablation device and at least a second mode of data of the multiple modes of data comprises the post-ablation arrhythmic ECG data.

10. The apparatus of claim 1, wherein predicting the determination of the pulmonary vein reconnection further comprises stratifying the patient into a subgroup for differential treatment planning.

11. A method for prediction of repeat ablation efficacy, the method comprising:
receiving, by at least a processor and from an electrocardiogram device, post-ablation arrhythmic electrocardiogram (ECG) data, wherein the electrocardiogram device is configured to detect the post-ablation arrhythmic ECG data representative of a post-ablation arrythmia of a patient who has previously undergone an ablation procedure;
predicting, by at the least a processor and using a repeat-ablation efficacy machine-learning model, a determination of a pulmonary vein reconnection by identifying features within the post-ablation arrhythmic ECG data that are historically representative of pulmonary vein reconnection determinations; and
transmitting, by the at least a processor and for display, the determination.

12. The method of claim 11, wherein the determination of the pulmonary vein reconnection comprises a predicted chance of pulmonary vein reconnection.

13. The method of claim 12, wherein predicting the determination of the pulmonary vein reconnection further comprises comparing the predicted chance against one or more predefined thresholds.

14. The method of claim 11, wherein the determination comprises a probability that a recurrent case of atrial fibrillation can be addressed through a repeat ablation procedure.

15. The method of claim 11, wherein the method further comprises:
generating, by the at least a processor, a treatment recommendation based on the determination; and
transmitting, for display, the treatment recommendation.

16. The method of claim 15, wherein the treatment recommendation comprises a secondary ablation procedure.

17. The method of claim 11, wherein the determination comprises a predicted efficacy of a repeat ablation procedure.

18. The method of claim 11, wherein the repeat-ablation efficacy machine-learning model comprises a multimodal model configured to receive multiple modes of data at once.

19. The method of claim 18, wherein at least a first mode of data of the multiple modes of data comprises ablation data received from an ablation device and at least a second mode of data of the multiple modes of data comprises the post-ablation arrhythmic ECG data.

20. The method of claim 11, wherein predicting the determination of the pulmonary vein reconnection further comprises stratifying the patient into a subgroup for differential treatment planning.
